(19) United States
(12) Patent Application Publication
(10) Pub. No.: US 2026/0308172 A1
(43) Pub. Date: Oct. 8, 2026

(54) TRICYCLIC COMPOUNDS FOR TREATMENT OF BACTERIAL INFECTIONS

(71) Applicant: Hoffmann-La Roche Inc., Little Falls, NJ (US)

(72) Inventors: Fabian DEY, Zuerich (CH); Xiao DING, Shanghai (CN); Christian LERNER, Bottmingen (CH); Houguang SHI, Shanghai (CN); Xuefei TAN, Shanghai (CN); Jun WU, Shanghai (CN); Jiamin ZHENG, Shanghai (CN)

(73) Assignee: Hoffmann-La Roche Inc., Little Falls, NJ (US)

(21) Appl. No.: 19/474,037

(22) PCT Filed: Apr. 11, 2024
(86) PCT No.: PCT/EP2024/059790

§ 371 Date: Oct. 9, 2025

(30) Foreign Application Priority Data
Apr. 12, 2023 (WO) PCT/CN2023/087848

(51) Int. Cl. C07D 498/04; A61K 31/5383; A61P 31/04

(52) U.S. Cl. CPC C07D 498/04; A61K 31/5383; A61P 31/04

(57) ABSTRACT

The present invention relates to compounds of formula (I), wherein R¹ to R³, X, Y, A and W are as described herein, and their pharmaceutically acceptable salt thereof, and compositions including the compounds and methods of using the compounds.

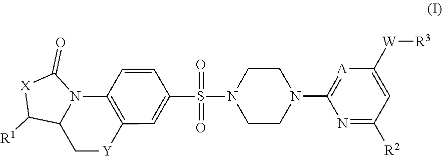

The present invention relates to organic compounds useful for the treatment and/or prevention of bacterial infections in a mammal. Specifically these molecules can inhibit the LPS synthesis pathway, in particular to inhibit LpxH, and are useful for treating bacterial infections.

### BACKGROUND OF THE INVENTION

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients. In hospital settings, an increasing number of strains of Staphylococcus aureus, Streptococcus pneumoniae, Enterococcus spp., Enterobacteriaceae such as Klebsiella pneumonia, Acinetobacter baumannii and Pseudomonas aeruginosa, major sources of infections, are becoming multi-drug resistant and therefore difficult to treat. This is particularly the case for Gram-negative organisms where the situation is getting worrisome since no novel agents with a differentiated mechanism of action have been approved for decades. Therefore, there is an important medical need for new antibacterial compounds addressing Gram-negative resistant bacteria, in particular third generation cephalosporins- and carbapenem-resistant Enterobacteriaceae and multi-drug-resistant Pseudomonas aeruginosa and Acinetobacter baumannii. One way to tackle the problem of cross-resistance to established classes of antibiotics is to inhibit an essential protein or function not targeted by current antibiotics.

Gram-negative bacteria are unique in that their outer membrane contains Lipopolysaccha-ride (LPS), which is crucial for maintaining membrane integrity, and is essential for bacterial viability (reviewed in Ann. Rev. Biochem 76:295-329, 2007). The major lipid component of LPS is Lipid A, and inhibition of Lipid A biosynthesis is lethal to bacteria. Lipid A is synthesized on the cytoplasmic surface of the bacterial inner membrane via a pathway that consists of nine different enzymes. These enzymes are highly conserved in most Gram-negative bacteria. LpxH, a calcineurin-like phosphatase (CLP), catalyzes the hydrolysis of UDP-2,3-diacyl-glucosamine (UDP-DAGn) to yield Lipid X and UMP (22, 24, 25). LpxH has no mammalian homologue, making it a good target for the development of novel antibiotics targeting Gram-negative bacteria.

### SUMMARY OF THE INVENTION

The present invention relates to novel compounds of formula (I),

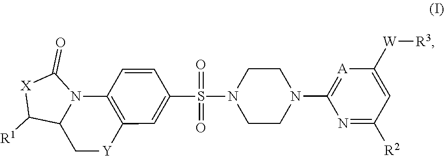

wherein
X is C₁₋₆alkylene, O, S or NH;
Y is C₁₋₆alkylene, O, S, SO, SO₂ or NH;
A is CH or N;
W is C₁₋₆alkylene, haloC₁₋₆alkylene, O, S, SO, SO₂ or NH;
R¹ is H, (aminoheterocyclyl)C₁₋₆alkyl, [(aminoC₁₋₆alkyl)heterocyclyl]C₁₋₆alkyl, amino, aminoC₁₋₆alkyl, hydroxyC₁₋₆alkyl or heterocyclylamino;
R² is amino, C₁₋₆alkyl, halogen, haloC₁₋₆alkyl, C₁₋₆alkoxy or haloC₁₋₆alkoxy;
R³ is (aminoC₁₋₆alkylamino)carbonylC₂₋₆alkynyl,
   C₃₋₇cycloalkyl substituted by (aminoC₁₋₆alkyl)₂aminocarbonyl, [(aminoC₁₋₆alkyl)pyrrolidiniumyl]C₁₋₆alkylamino, [(aminocarbonylC₁₋₆alkyl)pyrrolidiniumylC₁₋₆alkyl]amino, [aminocarbonylC₁₋₆alkyl(C₁₋₆alkyl)pyrrolidiniumylcarbonyl]amino, amino, aminoC₁₋₆alkylaminocarbonyl or aminoC₁₋₆alkylcarbonylamino,
   halogen,
   heterocyclyl unsubstituted or substituted by amino, C₁₋₆alkyl, aminoC₁₋₆alkylcarbonyl, (aminoC₁₋₆alkylcarbonyl)aminoC₁₋₆alkylcarbonyl, aminoC₁₋₆alkyl or aminocarbonylC₁₋₆alkyl, or
   aryl or heteroaryl unsubstituted or substituted by amino, C₁₋₆alkyl, halogen, haloC₁₋₆alkyl, C₁₋₆alkoxy or haloC₁₋₆alkoxy,
      or a pharmaceutically acceptable salt thereof.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, trifluoroethyl, fluoromethyl, difluoromethyl, difluoroethyl or trifluoromethyl.

The term “haloC₁₋₆alkylene” denotes a C₁₋₆alkylene group wherein at least one of the hydrogen atoms of the C₁₋₆alkylene group has been replaced by same or different halogen atoms, particularly fluoro atoms.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, or bicyclo[2.2.2]octanyl.

The term “aryl” denotes a monovalent aromatic carbocyclic mono- or bicyclic ring system comprising 6 to 10 carbon ring atoms. Examples of aryl moieties include phenyl and naphthyl. The term “arylene” denotes a divalent aryl group.

The term “heteroaryl” refers to any mono-, bi-, or tricyclic aromatic ring system containing from 1 to 4 heteroatoms selected from nitrogen, oxygen, and sulfur, and in an example embodiment, at least one heteroatom is nitrogen. See, for example, Lang's Handbook of Chemistry (Dean, J. A., ed.) 13th ed. Table 7-2 [1985]. Included in the definition are any bicyclic groups where any of the above heteroaryl rings are fused to an aryl ring, wherein the aryl ring or the heteroaryl ring is joined to the remainder of the molecule. In one embodiment, heteroaryl includes 5-6 membered monocyclic aromatic groups where one or more ring atoms is nitrogen, sulfur or oxygen. In one embodiment, heteroaryl includes 7-12 membered bicyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl, 1H-pyrazolo[3,4-d]pyrimidine, 1H-pyrazolo[3,4-d]pyridazine, imidazo[1,5-a]pyrazine, imidazo[5,1-f][1,2,4]triazine, [1,2,4]triazolo[4,3-a]pyrazine, 1H-pyrazolo[3,4-c]pyridazine, 1H-pyrazolo[3,4-b]pyridine, 1H-pyrazolo[4,3-d]pyrimidine, 1H-pyrazolo[3,4-c]pyridine, 1H-pyrazolo[4,3-c]pyridine and purinyl, as well as benzo-fused derivatives, for example benzoxazolyl, benzofuryl, benzothiazolyl, benzothiadiazolyl, benzotriazolyl, benzoimidazolyl, indazolyl and indolyl.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-11 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO2), and any nitrogen heteroatom may optionally be quaternized (e.g., [NR4]+Cl—, [NR4]+OH—). Examples of heterocycles include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl.

The term “cis” and “trans” denote the relative stereochemistry of the molecule or moiety. For example: Intermediate 1,

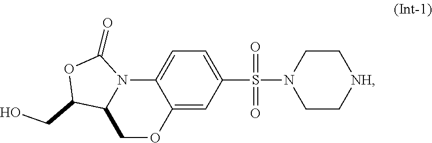 [SMILES: O=C1O[C@H](CO)[C@@H]2COc3cc(S(=O)(=O)N4CCNCC4)ccc3N12]

refers to a mixture of

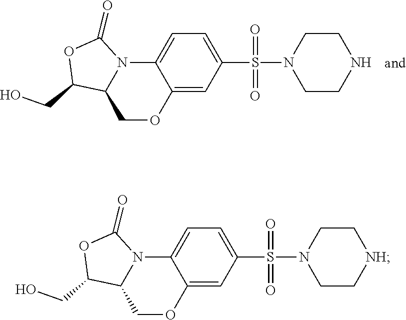 [SMILES: O=C1O[C@H](CO)[C@@H]2COc3cc(S(=O)(=O)N4CCNCC4)ccc3N12 ; O=C1O[C@@H](CO)[C@H]2COc3cc(S(=O)(=O)N4CCNCC4)ccc3N12]

similarly,

### Intermediate 3,

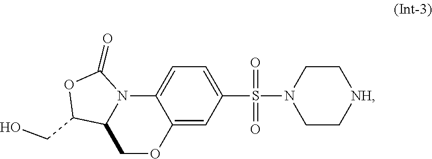 [SMILES: O=C1O[C@@H](CO)[C@@H]2COc3cc(S(=O)(=O)N4CCNCC4)ccc3N12]

refers to a mixture of

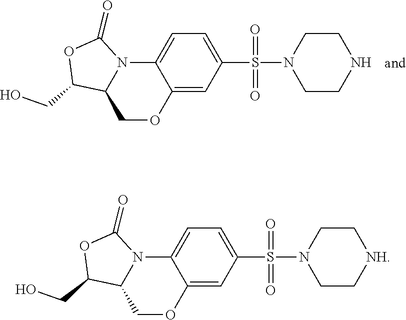 [SMILES: O=C1O[C@@H](CO)[C@@H]2COc3cc(S(=O)(=O)N4CCNCC4)ccc3N12 ; O=C1O[C@H](CO)[C@H]2COc3cc(S(=O)(=O)N4CCNCC4)ccc3N12]

The way of showing relative stereochemistry also applies to the final compounds.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Inhibitor of UDP-2,3-diacylglucosamine hydrolase (Lpxh)

The present invention relates to (i) a compound of formula (I),

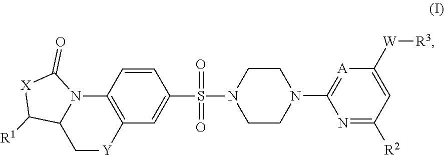

wherein
X is C₁₋₆alkylene, O, S or NH;
Y is C₁₋₆alkylene, O, S, SO, SO₂ or NH;
A is CH or N;
W is C₁₋₆alkylene, haloC₁₋₆alkylene, O, S, SO, SO₂ or NH;
R¹ is H, (aminoheterocyclyl)C₁₋₆alkyl, [(aminoC₁₋₆alkyl)heterocyclyl]C₁₋₆alkyl, amino, aminoC₁₋₆alkyl, hydroxyC₁₋₆alkyl or heterocyclylamino;
R² is amino, C₁₋₆alkyl, halogen, haloC₁₋₆alkyl, C₁₋₆alkoxy or haloC₁₋₆alkoxy;
R³ is (aminoC₁₋₆alkylamino)carbonylC₂₋₆alkynyl,
   C₃₋₇cycloalkyl substituted by (aminoC₁₋₆alkyl)₂aminocarbonyl, [(aminoC₁₋₆alkyl)pyrrolidiniumyl]C₁₋₆alkylamino, [(aminocarbonylC₁₋₆alkyl)pyrrolidiniumylC₁₋₆alkyl]amino, [aminocarbonylC₁₋₆alkyl(C₁₋₆alkyl)pyrrolidiniumylcarbonyl]amino, amino, aminoC₁₋₆alkylaminocarbonyl or aminoC₁₋₆alkylcarbonylamino,
   halogen,
   heterocyclyl unsubstituted or substituted by amino, C₁₋₆alkyl, aminoC₁₋₆alkylcarbonyl, (aminoC₁₋₆alkylcarbonyl)aminoC₁₋₆alkylcarbonyl, aminoC₁₋₆alkyl or aminocarbonylC₁₋₆alkyl, or
   aryl or heteroaryl unsubstituted or substituted by amino, C₁₋₆alkyl, halogen, haloC₁₋₆alkyl, C₁₋₆alkoxy or haloC₁₋₆alkoxy,
      or a pharmaceutically acceptable salt thereof.

A further embodiment of present invention is (ii) a compound of formula (I), wherein
X is C₁₋₆alkylene or O;
Y is O;
A is CH or N;
W is haloC₁₋₆alkylene, O or SO₂;
R¹ is H, (aminoazetidinyl)C₁₋₆alkyl, [(aminoC₁₋₆alkyl)azetidinyl]C₁₋₆alkyl, amino, aminoC₁₋₆alkyl, hydroxyC₁₋₆alkyl or piperidylamino;
R² is C₁₋₆alkyl or halogen;
R³ is (aminoC₁₋₆alkylamino)carbonylC₂₋₆alkynyl,
   1,4-dioxanyl,
   3,6-dihydro-2H-pyridinyl substituted by C₁₋₆alkyl,
   3-azabicyclo[3.1.0]hexanyl,
   3-oxabicyclo[3.1.0]hexanyl,
   azetidinyl substituted by aminoC₁₋₆alkylcarbonyl,
   C₃₋₇cycloalkyl substituted by (aminoC₁₋₆alkyl)₂aminocarbonyl, [(aminoC₁₋₆alkyl)pyrrolidiniumyl]C₁₋₆alkylamino, [(aminocarbonylC₁₋₆alkyl)pyrrolidiniumylC₁₋₆alkyl]amino, [aminocarbonylC₁₋₆alkyl(C₁₋₆alkyl)pyrrolidiniumylcarbonyl]amino, amino, aminoC₁₋₆alkylaminocarbonyl or aminoC₁₋₆alkylcarbonylamino,
   halogen,
   morpholinyl unsubstituted or substituted by C₁₋₆alkyl,
   phenyl substituted by amino, or
   piperidyl unsubstituted or substituted by (aminoC₁₋₆alkylcarbonyl)aminoC₁₋₆alkylcarbonyl, aminoC₁₋₆alkyl, aminoC₁₋₆alkylcarbonyl, aminocarbonylC₁₋₆alkyl or C₁₋₆alkyl;
      or a pharmaceutically acceptable salt thereof.

A further embodiment of present invention is (iii) a compound of formula (I) according to (i) or (ii), or a pharmaceutically acceptable salt thereof, wherein X is CH₂ or O.

A further embodiment of present invention is (iv) a compound of formula (I), according to any one of (i) to (iii), or a pharmaceutically acceptable salt thereof, wherein W is haloC₁₋₆alkylene.

A further embodiment of present invention is (v) a compound of formula (I), according to any one of (i) to (iv), wherein W is CF₂.

A further embodiment of present invention is (vi) a compound of formula (I), or a pharmaceutically acceptable salt thereof, according to any one of (i) to (v), wherein R¹ is (aminoazetidinyl)C₁₋₆alkyl, amino, aminoC₁₋₆alkyl or hydroxyC₁₋₆alkyl.

A further embodiment of present invention is (vii) a compound of formula (I), or a pharmaceutically acceptable salt thereof, according to any one of (i) to (vi), wherein R¹ is (3-aminoazetidin-1-yl)methyl, amino, aminomethyl or hydroxymethyl.

A further embodiment of present invention is (viii) a compound of formula (I), or a pharmaceutically acceptable salt thereof, according to any one of (i) to (vii), wherein R² is chloro or methyl.

A further embodiment of present invention is (ix) a compound of formula (I), or a pharmaceutically acceptable salt thereof, according to any one of (i) to (viii), wherein
R³ is 1,4-dioxanyl,
   3,6-dihydro-2H-pyridinyl substituted by C₁₋₆alkyl,
   3-azabicyclo[3.1.0]hexanyl,
   C₃₋₇cycloalkyl substituted by (aminoC₁₋₆alkyl)₂aminocarbonyl, [aminocarbonylC₁₋₆alkyl(C₁₋₆alkyl)pyrrolidiniumylcarbonyl]amino or aminoC₁₋₆alkylaminocarbonyl,
   halogen,
   morpholinyl, or
   piperidyl.

A further embodiment of present invention is (x) a compound of formula (I), or a pharmaceutically acceptable salt thereof, according to any one of (i) to (ix), wherein R³ is 1,4-dioxan-2-yl, 1-methyl-3,6-dihydro-2H-pyridin-4-yl, 3-azabicyclo[3.1.0]hexan-1-yl, 4-(3-aminopropylcarbamoyl)cyclohexyl, 4-(3-aminopropylcarbamoyl)cyclohexyl, 4-(3-aminopropylcarbamoyl)cyclohexyl, 4-[[1-(2-amino-2-oxo-ethyl)-1-methyl-pyrrolidin-1-ium-2-carbonyl]amino]cyclohexyl, 4-[bis(3-aminopropyl)carbamoyl]cyclohexyl, 4-piperidyl, fluoro or morpholin-2-yl.

A further embodiment of present invention is (xi) a compound of formula (I), wherein
X is C₁₋₆alkylene or O;
Y is O;
A is CH or N;
W is haloC₁₋₆alkylene;
R¹ is (aminoazetidinyl)C₁₋₆alkyl, amino, aminoC₁₋₆alkyl or hydroxyC₁₋₆alkyl;
R² is C₁₋₆alkyl or halogen;
R³ is 1,4-dioxanyl,
   3,6-dihydro-2H-pyridinyl substituted by C₁₋₆alkyl,
   3-azabicyclo[3.1.0]hexanyl,
   C₃₋₇cycloalkyl substituted by (aminoC₁₋₆alkyl)₂aminocarbonyl, [aminocarbonylC₁₋₆alkyl(C₁₋₆alkyl)pyrrolidiniumylcarbonyl]amino or aminoC₁₋₆alkylaminocarbonyl,
   halogen,
   morpholinyl, or
   piperidyl;
   or a pharmaceutically acceptable salt thereof.

A further embodiment of present invention is (xii) a compound of formula (I), or a pharmaceutically acceptable salt thereof, according to any one of (i) to (xi), wherein
X is CH₂ or O;
Y is O;
A is CH or N;
W is CF₂;
R¹ is (3-aminoazetidin-1-yl)methyl, amino, aminomethyl or hydroxymethyl;
R² is chloro or methyl;
R³ is 1,4-dioxan-2-yl, 1-methyl-3,6-dihydro-2H-pyridin-4-yl, 3-azabicyclo[3.1.0]hexan-1-yl, 4-(3-aminopropylcarbamoyl)cyclohexyl, 4-(3-aminopropylcarbamoyl)cyclohexyl, 4-(3-aminopropylcarbamoyl)cyclohexyl, 4-[[1-(2-amino-2-oxo-ethyl)-1-methyl-pyrrolidin-1-ium-2-carbonyl]amino]cyclohexyl, 4-[bis(3-aminopropyl)carbamoyl]cyclohexyl, 4-piperidyl, fluoro or morpholin-2-yl;
   or a pharmaceutically acceptable salt thereof.

Another embodiment of present invention is a compound of formula (I) selected from the following:
trans-3-(aminomethyl)-7-[4-[6-chloro-4-(trifluoromethyl)-2-pyridyl]piperazin-1-yl]sulfonyl-3a,4-dihydro-3H-oxazolo[4,3-c][1,4]benzoxazin-1-one;
trans-3-(aminomethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
trans-3-(aminomethyl)-7-[4-(6-chloro-4-cyclopropylsulfonyl-2-pyridyl) piperazin-1-yl]sulfonyl-3a,4-dihydro-3H-oxazolo[4,3-c][1,4]benzoxazin-1-one;
trans-3-(aminomethyl)-7-[4-[4-(4-aminophenyl)sulfonyl-6-chloro-2-pyridyl]piperazin-1-yl]sulfonyl-3a,4-dihydro-3H-oxazolo[4,3-c][1,4]benzoxazin-1-one;
cis-3-(aminomethyl)-7-[4-(6-chloro-4-cyclopropylsulfonyl-2-pyridyl) piperazin-1-yl]sulfonyl-3a,4-dihydro-3H-oxazolo[4,3-c][1,4]benzoxazin-1-one;
cis-3-(aminomethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
cis-3-(aminomethyl)-7-[4-[6-chloro-4-(trifluoromethyl)-2-pyridyl]piperazin-1-yl]sulfonyl-3a,4-dihydro-3H-oxazolo[4,3-c][1,4]benzoxazin-1-one;
3-Amino-7-[4-[6-chloro-4-[1,4-dioxan-2-yl(difluoro)methyl]-2-pyridyl]piperazin-1-yl]sulfonyl-2,3,3a,4-tetrahydropyrrolo[2,1-c][1,4]benzoxazin-1-one;
trans-3-(aminomethyl)-7-[4-[6-chloro-4-[difluoro(3-piperidyl)methyl]-2-pyridyl]piperazin-1-yl]sulfonyl-3a,4-dihydro-3H-oxazolo[4,3-c][1,4]benzoxazin-1-one;
cis-3-(aminomethyl)-7-[4-[6-chloro-4-[difluoro-(1-methyl-4-piperidyl)methyl]-2-pyridyl]piperazin-1-yl]sulfonyl-3a,4-dihydro-3H-oxazolo[4,3-c][1,4]benzoxazin-1-one;
7-[4-[4-Methyl-6-(trifluoromethyl)pyrimidin-2-yl]piperazin-1-yl]sulfonyl-2,3,3a,4-tetrahydropyrrolo[2,1-c][1,4]benzoxazin-1-one;
7-[4-[6-Chloro-4-(trifluoromethyl)-2-pyridyl]piperazin-1-yl]sulfonyl-2,3,3a,4-tetrahydropyrrolo[2,1-c][1,4]benzoxazin-1-one;
7-[4-[4-(4-Aminophenyl)sulfonyl-6-chloro-2-pyridyl]piperazin-1-yl]sulfonyl-2,3,3a,4-tetrahydropyrrolo[2,1-c][1,4]benzoxazin-1-one;
7-[4-[4-(4-Aminophenyl)sulfonyl-6-bromo-2-pyridyl]piperazin-1-yl]sulfonyl-2,3,3a,4-tetrahydropyrrolo[2,1-c][1,4]benzoxazin-1-one;
(3R,3aR)-3-(hydroxymethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
7-[4-[6-Chloro-4-[difluoro-(1-methyl-4-piperidyl)methyl]-2-pyridyl]piperazin-1-yl]sulfonyl-2,3,3a,4-tetrahydropyrrolo[2,1-c][1,4]benzoxazin-1-one;
trans-3-(aminomethyl)-7-[4-[6-chloro-4-[1,4-dioxan-2-yl(difluoro)methyl]-2-pyridyl]piperazin-1-yl]sulfonyl-3a,4-dihydro-3H-oxazolo[4,3-c][1,4]benzoxazin-1-one;
trans-3-(aminomethyl)-7-[4-[6-chloro-4-[difluoro(3-oxabicyclo[3.1.0]hexan-6-yl)methyl]-2-pyridyl]piperazin-1-yl]sulfonyl-3a,4-dihydro-3H-oxazolo[4,3-c][1,4]benzoxazin-1-one;
trans-3-(aminomethyl)-7-[4-[6-chloro-4-[difluoro-(2-methyl-4-pyridyl)methyl]-2-pyridyl]piperazin-1-yl]sulfonyl-3a,4-dihydro-3H-oxazolo[4,3-c][1,4]benzoxazin-1-one;
cis-3-(aminomethyl)-7-[4-[6-chloro-4-[difluoro-(1-methyl-3,6-dihydro-2H-pyridin-4-yl)methyl]-2-pyridyl]piperazin-1-yl]sulfonyl-3a,4-dihydro-3H-oxazolo[4,3-c][1,4]benzoxazin-1-one;
(3R,3aS)-3-(Aminomethyl)-7-[4-[6-chloro-4-[[(2S)-1,4-dioxan-2-yl]-difluoro-methyl]-2-pyridyl]piperazin-1-yl]sulfonyl-3a,4-dihydro-3H-oxazolo[4,3-c][1,4]benzoxazin-1-one;
(3S,3aR)-3-(aminomethyl)-7-[4-[6-chloro-4-[[(2S)-1,4-dioxan-2-yl]-difluoro-methyl]-2-pyridyl]piperazin-1-yl]sulfonyl-3a,4-dihydro-3H-oxazolo[4,3-c][1,4]benzoxazin-1-one;
(3S,3aS)-3-(aminomethyl)-7-[4-[6-chloro-4-[difluoro-[(2R)-morpholin-2-yl]methyl]-2-pyridyl]piperazin-1-yl]sulfonyl-3a,4-dihydro-3H-oxazolo[4,3-c][1,4]benzoxazin-1-one;
(3R,3aR)-3-(aminomethyl)-7-[4-[6-chloro-4-[difluoro-[(2R)-morpholin-2-yl]methyl]-2-pyridyl]piperazin-1-yl]sulfonyl-3a,4-dihydro-3H-oxazolo[4,3-c][1,4]benzoxazin-1-one;
(3R,3aS)-3-[(3-aminoazetidin-1-yl)methyl]-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
(3R,3aS)-3-[[3-(aminomethyl)azetidin-1-yl]methyl]-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
7-[4-[6-Chloro-4-[difluoro-[rac-(2S)-1,4-dioxan-2-yl]methyl]-2-pyridyl]piperazin-1-yl]sulfonyl-3-(4-piperidylamino)-2,3,3a,4-tetrahydropyrrolo[2,1-c][1,4]benzoxazin-1-one;
(3R,3aR)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3S,3aS)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
7-[4-[6-Chloro-4-[difluoro-[(2R)-morpholin-2-yl]methyl]-2-pyridyl]piperazin-1-yl]sulfonyl-2,3,3a,4-tetrahydropyrrolo[2,1-c][1,4]benzoxazin-1-one;
3-Amino-7-[4-[6-chloro-4-[difluoro-[(2R)-morpholin-2-yl]methyl]-2-pyridyl]piperazin-1-yl]sulfonyl-2,3,3a,4-tetrahydropyrrolo[2,1-c][1,4]benzoxazin-1-one;
cis-7-[4-[6-chloro-4-[difluoro-[(2R)-morpholin-2-yl]methyl]-2-pyridyl]piperazin-1-yl]sulfonyl-3-(hydroxymethyl)-3a,4-dihydro-3H-oxazolo[4,3-c][1,4]benzoxazin-1-one;
cis-7-[4-[6-Chloro-4-[difluoro(3-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[3-azabicyclo[3.1.0]hexan-6-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[azetidin-3-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[3-azabicyclo[3.1.0]hexan-1-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3S,3aR)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aS)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[2-azaspiro[3.3]heptan-6-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aS)-7-[4-[6-chloro-4-[difluoro-(5-methylmorpholin-2-yl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(2-aminopropanoyl)azetidin-3-yl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-(4-aminocyclohexoxy)-6-chloro-2-pyridyl]piperazin-1-yl]sulfonyl-3-(hydroxymethyl)-3a,4-dihydro-3H-oxazolo[4,3-c][1,4]benzoxazin-1-one;
3-Amino-7-[4-[4-(4-aminocyclohexoxy)-6-chloro-2-pyridyl]piperazin-1-yl]sulfonyl-2,3,3a,4-tetrahydropyrrolo[2,1-c][1,4]benzoxazin-1-one;
(E)-4-[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-N-(3-aminopropyl)-4,4-difluoro-but-2-enamide;
4-[[2-[4-[(3-Amino-1-oxo-2,3,3a,4-tetrahydropyrrolo[2,1-c][1,4]benzoxazin-7-yl)sulfonyl]piperazin-1-yl]-6-chloro-4-pyridyl]-difluoro-methyl]-N-(3-aminopropyl)cyclohexanecarboxamide;
4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-(3-aminopropyl)cyclohexanecarboxamide;
4-[[2-[4-[[cis-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-(3-aminopropyl)cyclohexanecarboxamide;
4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N,N-bis(3-aminopropyl)cyclohexanecarboxamide;
4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-(3-aminopropyl)cyclohexanecarboxamide;
trans-7-[4-[6-chloro-4-[difluoro-[(2S)-morpholin-2-yl]methyl]-2-pyridyl]piperazin-1-yl]sulfonyl-3-(hydroxymethyl)-3a,4-dihydro-3H-oxazolo[4,3-c][1,4]benzoxazin-1-one;
N-[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]oxy]cyclohexyl]-3-amino-propanamide;
(3R,3aR)-7-[4-[4-[[1-[2-[bis(2-aminoethyl)amino]acetyl]-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(2-aminoacetyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(3-aminopropanoyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(2-aminoethyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]acetamide;
N-[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]-2-oxo-ethyl]-2-amino-acetamide;
(1S,2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(1R,2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(1S,2S)—N-[4-[[2-[4-[[(3S,3aR)-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(1R,2S)—N-[4-[[2-[4-[[(3S,3aR)-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(2S)-1-(2-aminoethyl)-N-[4-[[2-chloro-6-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-4-pyridyl]-difluoro-methyl]cyclohexyl]-1-methyl-pyrrolidin-1-ium-2-carboxamide;chloride;
4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-amino-2-oxo-ethyl)pyrrolidin-1-ium-1-yl]ethyl]cyclohexanecarboxamide;chloride; and
4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-aminoethyl)pyrrolidin-1-ium-1-yl]ethyl]cyclohexanecarboxamide;chloride;
   or a pharmaceutically acceptable salt thereof.

Another embodiment relates to a process for the preparation of a compound according to any one of (i) to (xiii) comprising any of the following steps:
a) formation of compound of formula (I) via substitution reaction between compound of formula (VII),

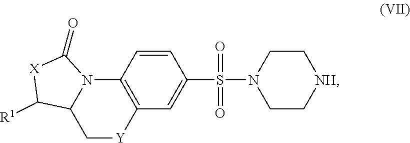

and compound of formula (VIII),

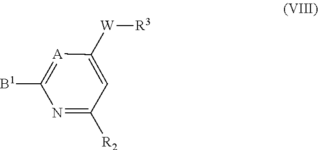

in the presence of a base;
b) formation of compound of formula (XVII),

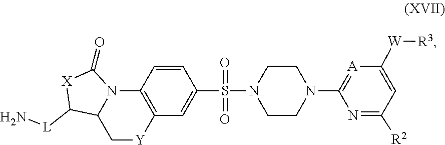

by deprotection of compound of formula (XVI),

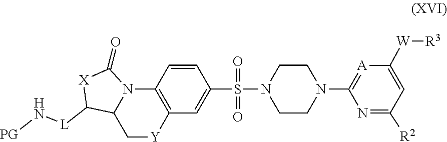

in the presence of an acid or a reducing reagent;
wherein
in step a) the base is DIEA;
in step b), the acid is trifluoroacetic acid; the reducing reagent is palladium on carbon in the presence of H₂;
wherein L is C₁₋₆alkylene or a bond; PG is tert-butoxycarbonyl or benzyloxycarbonyl; R¹ to R³, X, Y, A and W are defined as in any one of (i) to (xii).

A further embodiment of present invention is (xv) a compound or pharmaceutically acceptable salt according to any one of (i) to (xiii) for use as therapeutically active substance.

A further embodiment of present invention is (xvi) a pharmaceutical composition comprising a compound in accordance with any one of claims (i) to (xiii) and a therapeutically inert carrier.

A further embodiment of present invention is (xvii) the use of a compound according to any one of (i) to (xiii) for the inhibition of LpxH.

A further embodiment of present invention is (xviii) the use of a compound according to any one of (i) to (xiii) for the treatment or prophylaxis of bacterial infection, particularly the bacteria is gram-negative bacteria.

A further embodiment of present invention is (xix) the use of a compound according to any one of (i) to (xiii) for the preparation of a medicament for the treatment or prophylaxis of bacterial infection, particularly the bacteria is gram-negative bacteria.

A further embodiment of present invention is (xx) the use of a compound according to (xviii) or (xix), wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

A further embodiment of present invention is (xxi) the use of a compound according to (xx), wherein the gram-negative bacteria is Enterobacteriaceae, wherein Enterobacteriaceae is Klebsiella pneumoniae or Escherichia coli.

A further embodiment of present invention is (xxii) a compound or pharmaceutically acceptable salt according to any one of (i) to (xiii) for use in the treatment or prophylaxis of bacterial infection, particularly the bacteria is gram-negative bacteria.

A further embodiment of present invention is (xxiii) a compound according to (xxii), wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

A further embodiment of present invention is (xxiv) a compound according to (xxiii), wherein the gram-negative bacteria is Enterobacteriaceae, wherein Enterobacteriaceae is Klebsiella pneumoniae or Escherichia coli.

A further embodiment of present invention is (xxv) a compound or pharmaceutically acceptable salt according to any one of (i) to (xiii), when manufactured according to a process of (xiv).

A further embodiment of present invention is (xxvi) a method for the treatment or prophylaxis of bacterial infection, particularly the bacteria is gram-negative bacteria, which method comprises administering a therapeutically effective amount of a compound as defined in any one of (i) to (xiii), to a patient in need thereof.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduced bacterial load or improve host survival through the inhibition of Lipid A biosynthesis by targeting LpxH enzyme. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 1 to 100 mg/kg of patient body weight per day, with the typical initial range of compound used being 0.3 to 15 mg/kg/day. In another embodiment, oral unit dosage forms, such as tablets and capsules, preferably contain from about 5 to about 5000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 10 to 500 mg of the compound of the invention compounded with about 40 to 400 mg anhydrous lactose, about 5 to 50 mg sodium croscarmellose, about 5 to 50 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 1000 mg) of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I), or a stereoisomer or pharmaceutically acceptable salt thereof. In a further embodiment includes a pharmaceutical composition comprising a compound of Formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment and/or prevention of bacterial infections.

The following composition A and B illustrate typical compositions of the present invention, but serve merely as representative thereof.

Composition A
A compound of the present invention can be used in a
manner known per se as the active ingredient for the
production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

Composition B
A compound of the present invention can be used in a
manner known per se as the active ingredient for the
production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

### Indications and Methods of Treatment

The compounds of the invention are inhibitors of the LpxH enzyme, a key enzyme of the LPS synthesis pathway that is essential in most gram-negative bacteria. Accordingly, the compounds of the invention can prevent bacterial growth of susceptible organisms and are useful for: preventing or treating a bacterial infection, preferably a Gram-negative bacterial infection (all claimed) e.g. nosocomial pneumonia, urinary tract infections, systemic infections (bacteraemia and sepsis), skin and soft tissue infections, surgical infections, eye infections, intraabdominal infections, lung infections and diabetic foot infections caused by Gram-negative bacteria e.g. third generation cephalosporins- and carbapenem-resistant Enterobacteriaeceae (e.g. Klebsiella pneumoniae, Escherichia coli) and multi-drug-resistant Pseudomonas aeruginosa and Acinetobacter baumannii or Acinetobacter spp., e.g. Neisseria gonorrhoeae, Haemophilus influenzae, Helicobacter pylorus e.g. Bacteroides spp. e.g. Bacteroides fragilis, Bacteroides thetaiotaomicron, Bacteroides distasonis, Campylobacter jejuni, Campylobacter fetus or Campylobacter coil, Francisella tularensis and Providencia spp. e.g. Providencia stuartii, Providencia rettgeri or Providencia alcalifaciens and Pseudomonas spp.; and for cleaning purposes e.g. to remove pathogenic microbes and bacteria from surgical instruments, catheters and artificial implants or to make a room or an area aseptic.

The products of the invention can be administered, for example, parenterally e.g. by injection, or administered orally, perorally, such as in the form of tablets, coated tablets, dragees, hard and soft gelatin capsules, solutions, emulsions or suspensions, or rectally, such as in the form of suppositories. Pharmaceutical compositions containing these compounds can be prepared using conventional procedures familiar to those skilled in the art, such as by combining the ingredients into a dosage form together with suitable, non-toxic, inert, therapeutically compatible solid or liquid carrier materials and, if desired, the usual pharmaceutical adjuvants. It is contemplated that the compounds are ultimately embodied into compositions of suitable oral, parenteral or topical dosage forms. The compositions of this invention can contain, as optional ingredients, any of the various adjuvants, which are used ordinarily in the production of pharmaceutical preparations. Thus, for example, in formulating the present compositions into the desired oral dosage forms, one may use, as optional ingredients, fillers, such as co-precipitated aluminum hydroxide-calcium carbonate, di-calcium phosphate or lactose; disintegrating agents such as maize starch; and lubricating agents, such as talc, calcium stearate, and the like. It should be fully understood, however, that the optional ingredients herein named are given by way of example only and that the invention is not restricted to the use hereof. Other such adjuvants, which are well known in the art, can be employed in carrying out this invention. Suitable as such carrier materials are not only inorganic, but also organic carrier materials. Thus, for tablets, coated tablets, dragees and hard gelatin capsules there can be used, for example, lactose, maize starch or derivatives thereof, talc, stearic acid or its salts. Suitable carriers for soft gelatin capsules are, for example, vegetable oils, waxes, fats and semi-solid and liquid polyols (depending on the nature of the active substance; no carriers are, however, required in the case of soft gelatin capsules). Suitable carrier materials for the preparation of solutions and syrups are, for example, water, polyols, saccharose, invert sugar and glucose. Suitable carrier materials for suppositories are, for example, natural or hardened oils, waxes, fats and semi-liquid or liquid polyols. As pharmaceutical adjuvants there are contemplated the usual preservatives, solubilizers, stabilizers, wetting agents, emulsifiers, sweeteners, colorants, flavorings, salts for varying the osmotic pressure, buffers, coating agents and antioxidants.

### Synthesis

The compounds of the present invention can be prepared by any conventional means. Suitable processes for synthesizing these compounds as well as their starting materials are provided in the schemes below and in the examples. All substituents, in particular, R¹ to R³, A, W, X, and Y are defined as above unless otherwise indicated. Furthermore, and unless explicitly otherwise stated, all reactions, reaction conditions, abbreviations and symbols have the meanings well known to a person of ordinary skill in the art.

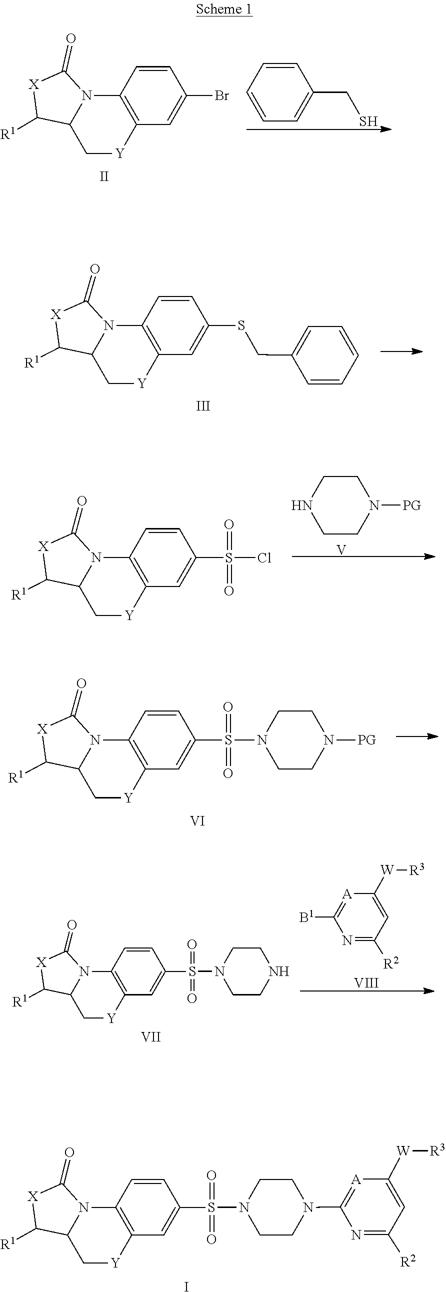

Wherein PG is a protecting group, such as tert-butoxycarbonyl and benzyloxycarbonyl group; B¹ is halogen.

Compound of formula (III) could be obtained from a palladium catalyzed cross coupling reaction between compound of formula (II) and phenylmethanethiol. Oxidative chlorination of compound of formula (III) affords arenesulfonyl chlorides (IV) in the presence of 1,3-dichloro-5,5-dimethylhydantoin. The reaction between compound of formula (IV) and compound of formula (V) gives compound of formula (VI) using a suitable base, such as DIEA. Compound (VII) can be obtained by the deprotection of compound of formula (VI) with a suitable acid such as trifluoroacetic acid. The nucleophilic substitution between compound of formula (VII) and compound of formula (VIII) affords compound of formula (I) in the presence of a base, such as DIEA.

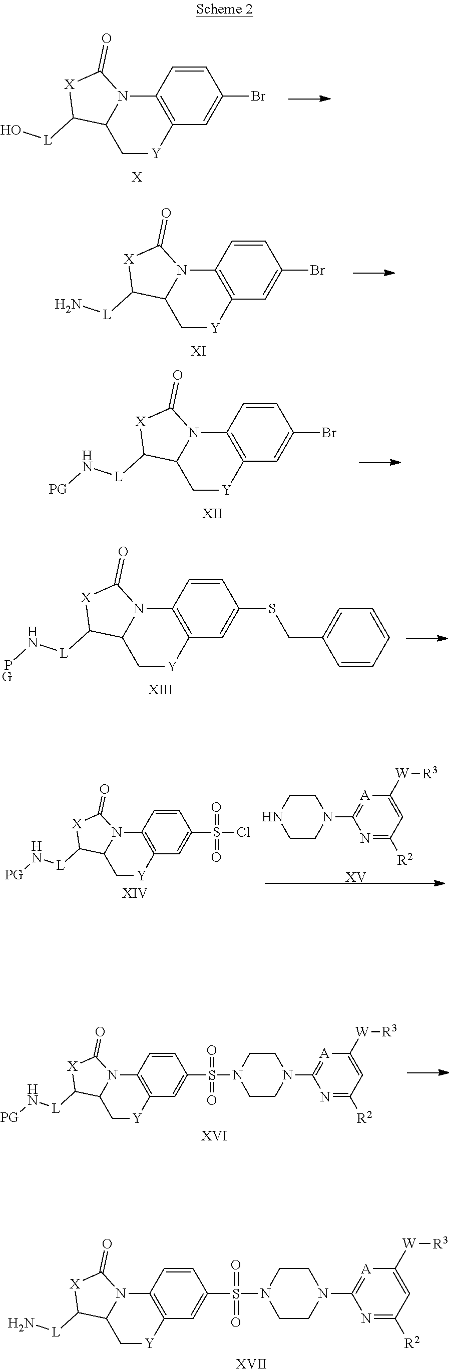

Wherein PG is a protecting group, such as tert-butoxycarbonyl and benzyloxycarbonyl group; L is C₁₋₆alkylene or a bond.

Amination of compound of formula (X) using trifluoromethanesulfonic anhydride and ammonia in the presence of suitable bases such as DIEA affords compound of formula (XI). The amine (XI) is protected by a suitable protecting group to give compound of formula (XII). Compound of formula (XIII) can be obtained from a palladium catalyzed cross coupling reaction between compound of formula (XII) and phenylmethanethiol. Oxidative chlorination of compound of formula (XIII) affords arenesulfonyl chlorides (XIV) in the presence of 1,3-dichloro-5,5-dimethylhydantoin. The nucleophilic substitution between compound of formula (XIV) and compound of formula (XV) gives compound of formula (XVI) in the presence of a suitable base such as DIEA, which is converted to compound of formula (XVII) by deprotection using a suitable acid such as trifluoroacetic acid, or a reducing reagent such as palladium on carbon in the presence of H₂.

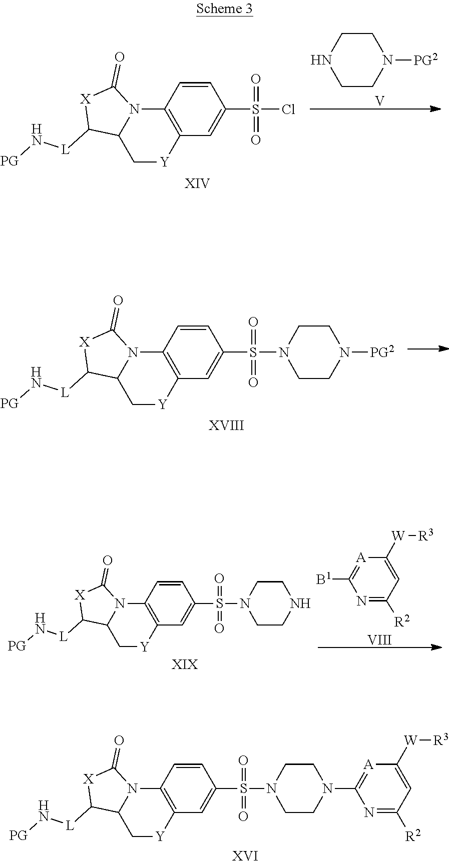

Wherein PG and PG² are independent protecting group, such as tert-butoxycarbonyl and benzyloxycarbonyl group; B¹ is halogen; L is C₁₋₆alkylene or a bond.

Compound of formula (XVI) can also be prepared according to scheme 3. The reaction between compound of formula (XIV) and compound of formula (V) gives compound of formula (XVIII) in the presence of a suitable base such as DIEA. Compound of formula (XVIII) is converted to compound of formula (XIX) by deprotection using a suitable acid such as trifluoroacetic acid, or a reducing reagent such as palladium on carbon in the presence of H₂. Compound of formula (XVI) can be obtained from the nucleophilic substitution between compound of formula (XIX) and compound of formula (VIII) in the presence of a suitable base, such as DIEA.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising any of the following steps:
a) formation of compound of formula (I) via substitution reaction between compound of formula (VII),

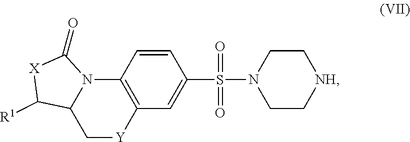

and compound of formula (VIII),

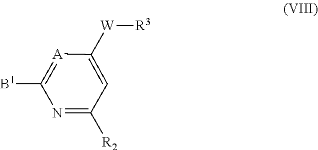

in the presence of a base;
b) formation of compound of formula (XVII),

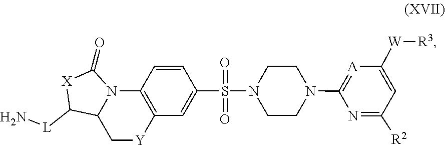

by deprotection of compound of formula (XVI),

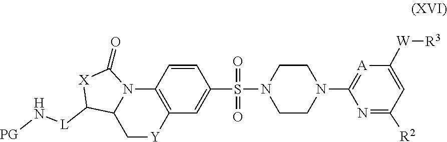

in the presence of an acid or a reducing reagent;
in step a) the base can be, for example, DIEA;
in step b), the acid can be, for example, trifluoroacetic acid; the reducing reagent can be, for example, palladium on carbon in the presence of H₂;
wherein L is C₁₋₆alkylene or a bond; PG is a protecting group, such as tert-butoxycarbonyl or benzyloxycarbonyl; R¹ to R³, X, Y, A and W are defined as above.

A compound of formula (I) when manufactured according to the above process is also an object of the invention.

### Examples

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
aq.: aqueous
DCM: dichloromethane
DIPEA: N,N-Diisopropylethylamine
DMF: N,N-dimethylformamide
DMSO: dimethyl sulfoxide
EtOAc: ethyl acetate
FA: formic acid
IPA: isopropyl alcohol
TFA: trifluoroacetic acid
TEMPO: 2,2,6,6-tetramethylpiperidinooxy
NMP: N-methyl-2-pyrrolidone
m-CPBA: 3-chloroperoxybenzoic acid
HATU: 1-[bis(dimethylamino)methylene]-1H-1,2,3-triazolo[4,5-b]pyridinium 3-oxide hexafluorophosphate
IC₅₀: the molar concentration of an inhibitor, which produces 50% of the maximum possible response for that inhibitor.
HPLC: high performance liquid chromatography
MS (ESI): mass spectroscopy (electron spray ionization)
MeCN: acetonitrile
MTBE: methyl tert-butyl ether
obsd: observed
PE: petroleum ether
TEA: triethylamine
δ: chemical shift
SFC: supercritical fluid chromatography

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400&500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

The following examples are intended to illustrate the meaning of the present invention but should by no means represent a limitation within the meaning of the present invention:

### Intermediate 1: cis-3-(Hydroxymethyl)-7-piperazin-1-ylsulfonyl-3a,4-dihydro-3H-oxazolo[4,3-c][1,4]benzoxazin-1-one

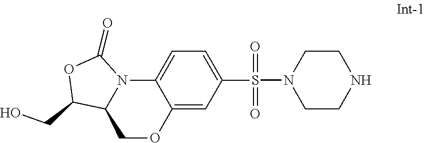 [SMILES: O=C1O[C@H](CO)[C@@H]2COc3cc(S(=O)(=O)N4CCNCC4)ccc3N12]

To a solution of tert-butyl 4-[[cis-3-(hydroxymethyl)-1-oxo-3a,4-dihydro-3H-oxazolo[4,3-c][1,4]benzoxazin-7-yl]sulfonyl]piperazine-1-carboxylate (1940 mg, 4.13 mmol, purchased from Shanghai DepuBiosciences Co., Ltd, Cat. number: 20200409-01) in DCM (20 mL) was added TFA (10.0 mL, 135 mmol) at room temperature. The mixture was stirred at room temperature for 2 h. After the reaction was completed, the mixture was concentrated in vacuo to give crude product, which was purified by prep-HPLC (NH₃ as additive) to give Int-1 (1400 mg, 91.72%) as a yellow solid. MS (ESI⁺) obsd. [(M+H)⁺]: 370.1.

### Intermediate 2A and Intermediate 2B: (3S,3aS)-3-(hydroxymethyl)-7-piperazin-1-ylsulfonyl-3a, 4-dihydro-3H-oxazolo[4,3-c][1,4]benzoxazin-1-one (Int-2A) and (3R,3aR)-3-(hydroxymethyl)-7-piperazin-1-ylsulfonyl-3a,4-dihydro-3H-oxazolo[4,3-c][1,4]benzoxazin-1-one (Int-2B)

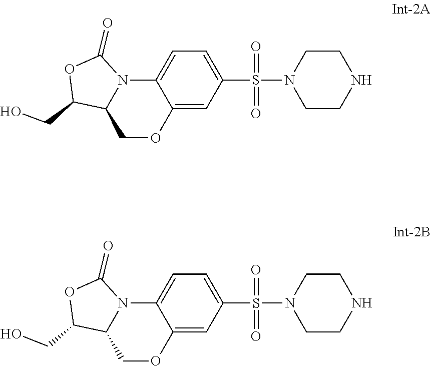 [SMILES: O=C1O[C@H](CO)[C@@H]2COc3cc(S(=O)(=O)N4CCNCC4)ccc3N12 ; O=C1O[C@@H](CO)[C@H]2COc3cc(S(=O)(=O)N4CCNCC4)ccc3N12]

### Step 1: tert-Butyl 4-[[(3S,3aS)-3-(hydroxymethyl)-1-oxo-3a,4-dihydro-3H-oxazolo[4,3-c][1, 4]benzoxazin-7-yl]sulfonyl]piperazine-1-carboxylate (Int-2-a) and tert-butyl 4-[[(3R,3aR)-3-(hydroxymethyl)-1-oxo-3a,4-dihydro-3H-oxazolo[4,3-c][1,4]benzoxazin-7-yl]sulfonyl]piperazine-1-carboxylate (Int-2-b)

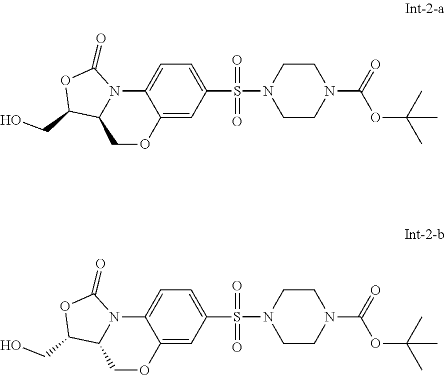 [SMILES: CC(C)(C)OC(=O)N1CCN(S(=O)(=O)c2ccc3c(c2)OC[C@H]2[C@@H](CO)OC(=O)N32)CC1 ; CC(C)(C)OC(=O)N1CCN(S(=O)(=O)c2ccc3c(c2)OC[C@@H]2[C@H](CO)OC(=O)N32)CC1]

### Int-2-b

Int-2-a and Int-2-b were obtained from chiral separation of tert-butyl 4-[[cis-3-(hydroxymethyl)-1-oxo-3a, 4-dihydro-3H-oxazolo[4,3-c][1,4]benzoxazin-7-yl]sulfonyl]piperazine-1-carboxylate by SFC (Instrument: SFC 80, Column: OX, 250×20 mm I.D., 5 μm. Mobile phase: A for CO₂ and B for ethanol (0.1% NH₃H₂O), Gradient: B 40% Flow rate: 40 mL/min, Back pressure: 100 bar, Column temperature: 35° C.). Int-2-a (faster eluted) showed its optical rotatory dispersion ([α]²⁵_{D}=62.354). Int-2-b (slower eluted) showed its optical rotatory dispersion ([α]²⁵_{D}=−111.337).

### Step 2: (3S,3aS)-3-(hydroxymethyl)-7-piperazin-1-ylsulfonyl-3a,4-dihydro-3H-oxazolo[4,3-c][1,4]benzoxazin-1-one (Int-2A) and (3R,3aR)-3-(hydroxymethyl)-7-piperazin-1-ylsulfonyl-3a,4-dihydro-3H-oxazolo[4,3-c][1,4]benzoxazin-1-one (Int-2B)

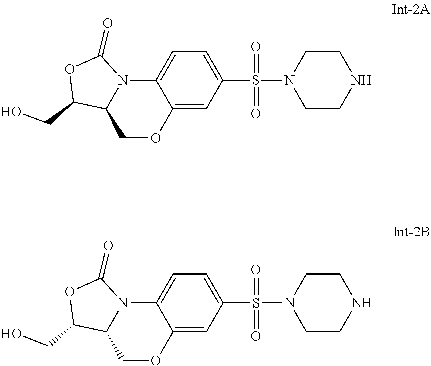 [SMILES: O=C1O[C@H](CO)[C@@H]2COc3cc(S(=O)(=O)N4CCNCC4)ccc3N12 ; O=C1O[C@@H](CO)[C@H]2COc3cc(S(=O)(=O)N4CCNCC4)ccc3N12]

A mixture of Int-2-a (0.100 g, 213 μmol) and TFA (1.13 g, 8.71 mmol) in DCM (1.5 mL) was stirred at 25° C. for 1 h. After the reaction was completed, the mixture was concentrated in vacuo and purified by prep-HPLC (NH₃ as additive) to give Int-2A as a yellow solid (78.7 mg, 100%). MS (ESI⁺) obsd. [(M+H)⁺]: 370.0.

A mixture of Int-2-b ([α]²⁵_{D}=−111.337) (0.105 g, 224 μmol) and TFA (1.27 g, 11.2 mmol) in DCM (1.5 mL) was stirred at 25° C. for 1 h. After the reaction was completed, the mixture was concentrated in vacuo to give crude, which was purified by prep-HPLC (NH₃ as additive) to give Int-2B as a yellow solid (78 mg, 94%). MS obsd. (ESI⁺) [(M+H)]⁺: 370.0.

### Intermediate 3: trans-3-(Hydroxymethyl)-7-piperazin-1-ylsulfonyl-3a,4-dihydro-3H-oxazolo[4,3-c][1,4]benzoxazin-1-one

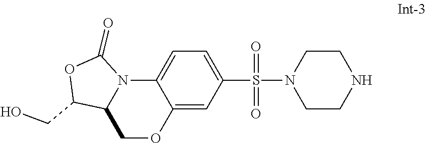 [SMILES: O=C1O[C@@H](CO)[C@@H]2COc3cc(S(=O)(=O)N4CCNCC4)ccc3N12]

The title compound was prepared in analogy to the preparation of Int-1 by using tert-butyl 4-[[trans-3-(hydroxymethyl)-1-oxo-3a,4-dihydro-3H-oxazolo[4,3-c][1,4]benzoxazin-7-yl]sulfonyl]piperazine-1-carboxylate (purchased from Shanghai DepuBiosciences Co., Ltd, Cat. number: 20200409-02) instead of tert-butyl 4-[[cis-3-(hydroxymethyl)-1-oxo-3a,4-dihydro-3H-oxazolo[4,3-c][1,4]benzoxazin-7-yl]sulfonyl]piperazine-1-carboxylate. Int-3 (118 mg) was obtained as a yellow solid. MS obsd. (ESI⁺) [(M+H)]⁺: 370.1.

### Intermediate 4A and Intermediate 4B: (3R,3aS)-3-(hydroxymethyl)-7-piperazin-1-ylsulfonyl-3a,4-dihydro-3H-oxazolo[4,3-c][1,4]benzoxazin-1-one (Int-4A) and (3S,3aR)-3-(hydroxymethyl)-7-piperazin-1-ylsulfonyl-3a,4-dihydro-3H-oxazolo[4,3-c][1,4]benzoxazin-1-one (Int-4B)

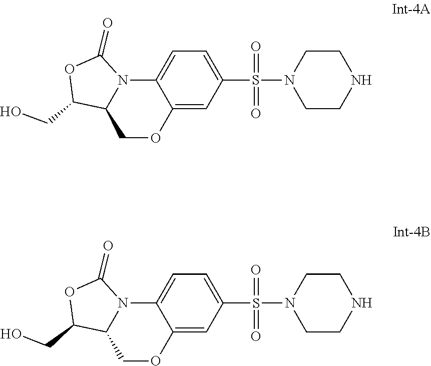 [SMILES: O=C1O[C@@H](CO)[C@@H]2COc3cc(S(=O)(=O)N4CCNCC4)ccc3N12 ; O=C1O[C@H](CO)[C@H]2COc3cc(S(=O)(=O)N4CCNCC4)ccc3N12]

### Step 1: tert-Butyl 4-[[(3R,3aS)-3-(hydroxymethyl)-1-oxo-3a,4-dihydro-3H-oxazolo[4,3-c][1,4]benzoxazin-7-yl]sulfonyl]piperazine-1-carboxylate (Int-4-a) and tert-butyl 4-[[(3S,3aR)-3-(hydroxymethyl)-1-oxo-3a,4-dihydro-3H-oxazolo[4,3-c][1,4]benzoxazin-7-yl]sulfonyl]piperazine-1-carboxylate (Int-4-b)

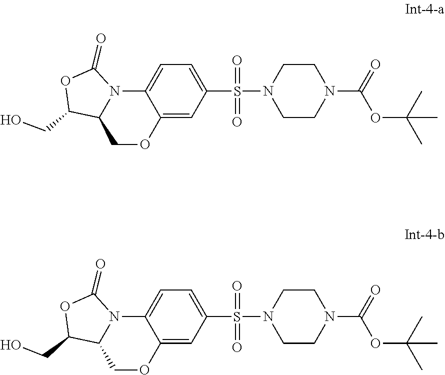 [SMILES: CC(C)(C)OC(=O)N1CCN(S(=O)(=O)c2ccc3c(c2)OC[C@H]2[C@H](CO)OC(=O)N32)CC1 ; CC(C)(C)OC(=O)N1CCN(S(=O)(=O)c2ccc3c(c2)OC[C@@H]2[C@@H](CO)OC(=O)N32)CC1]

Int-4-a (faster eluted) and Int-4-b (slower eluted) were obtained from chiral separation of tert-butyl 4-[[trans-3-(hydroxymethyl)-1-oxo-3a,4-dihydro-3H-oxazolo[4,3-c][1,4]benzoxazin-7-yl]sulfonyl]piperazine-1-carboxylate by SFC (Instrument: SFC 80, Column: IC, 250×30 mm I.D., 5 μm. Mobile phase: A for CO₂ and B for IPA (0.1% NH₃H₂O), Gradient: B 50% Flow rate: 50 mL/min, Back pressure: 100 bar Column temperature: 35° C.).

### Step 2: (3R,3aS)-3-(hydroxymethyl)-7-piperazin-1-ylsulfonyl-3a,4-dihydro-3H-oxazolo[4,3-c][1,4]benzoxazin-1-one (Int-4A) and (3S,3aR)-3-(hydroxymethyl)-7-piperazin-1-ylsulfonyl-3a,4-dihydro-3H-oxazolo[4,3-c][1,4]benzoxazin-1-one (Int-4B)

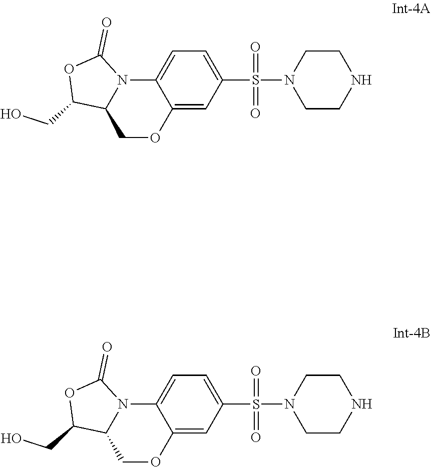 [SMILES: O=C1O[C@@H](CO)[C@@H]2COc3cc(S(=O)(=O)N4CCNCC4)ccc3N12 ; O=C1O[C@H](CO)[C@H]2COc3cc(S(=O)(=O)N4CCNCC4)ccc3N12]

A mixture of Int-4-a (60.2 mg, 0.128 mmol) and TFA (219.48 mg, 1.92 mmol) in DCM (1.5 mL) was stirred at room temperature for 1 h. After the reaction was completed, the mixture was concentrated in vacuo to give crude product, which was purified by prep-HPLC (NH₃ as additive) to give Int-4A as a yellow solid (47 mg, 100%). MS obsd. (ESI⁺) [(M+H)]⁺: 370.3.

A mixture of Int-4-b (60 mg, 0.128 mmol) and TFA (218.56 mg, 1.92 mmol) in DCM (1.5 mL) was stirred at rt for 1 h. The mixture was concentrated in vacuo and purified by prep-HPLC (NH₃ as additive) to give Int-4B as a yellow solid (47 mg, 100%). MS obsd. (ESI⁺) [(M+H)]⁺: 370.3.

### Intermediate 5: tert-Butyl N-[[cis-7-chlorosulfonyl-1-oxo-3a,4-dihydro-3H-oxazolo[4,3-c][1,4]benzoxazin-3-yl]methyl]carbamate

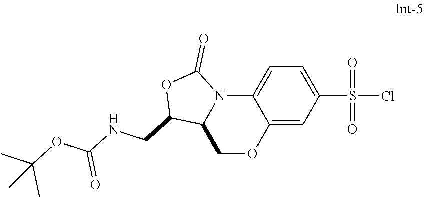 [SMILES: CC(C)(C)OC(=O)NC[C@H]1OC(=O)N2c3ccc(S(=O)(=O)Cl)cc3OC[C@@H]12]

### Step 1: (E)-4-(5-bromo-2-nitro-phenoxy) but-2-en-1-ol

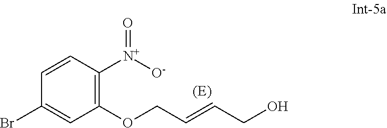 [SMILES: O=[N+]([O-])c1ccc(Br)cc1OC/C=C/CO]

To a mixture of sodium hydride (7.27 g, 181.82 mmol) in THF (200 mL) was added (E)-but-2-ene-1,4-diol (8.01 g, 90.91 mmol) slowly at 0° C. and the mixture was stirred at 25° C. for 0.5 h. Then 4-bromo-2-fluoro-1-nitro-benzene (10.0 g, 45.45 mmol) was added to the mixture and stirred at 25° C. for 3 h. After the reaction was completed, the mixture was poured into water (200.0 mL) and extracted with DCM (200 mL×3). The organic layer was washed with brine (100 mL×4), dried over anhydrous sodium sulfate, filtered and the filtrate was concentrated in vacuo to give crude product, which was purified by silica-gel chromatography (elute with EtOAc:PE=0-30%) to give Int-5a (8.5 g, 64.91% yield) as yellow solid.

### Step 2: (E)-4-(2-amino-5-bromo-phenoxy) but-2-en-1-ol (Int-5b)

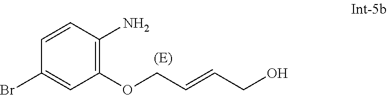 [SMILES: Nc1ccc(Br)cc1OC/C=C/CO]

To a mixture of Int-5a (8.5 g, 29.5 mmol) and acetic acid (28.35 g, 472.07 mmol) in THF (60 mL) was added zinc (16.0 g, 244.72 mmol) slowly at 25° C. After addition, the mixture was stirred at 25° C. for 4 h. After the reaction was completed, the mixture was diluted with EtOAc (500 mL) and filtered. The filtrate was adjusted to pH 8.0 with aq.NaHCO₃. The organic layer was washed with brine (100 mL×2), dried over anhydrous sodium sulfate and then filtered. The filtrate was concentrated in vacuo to give crude Int-5b (7.5 g, 98.48% yield) as light oil. MS (ESI⁺) obsd. [(M+H)⁺]: 257.8.

### Step 3: Benzyl N-[4-bromo-2-[(E)-4-hydroxybut-2-enoxy]phenyl]carbamate

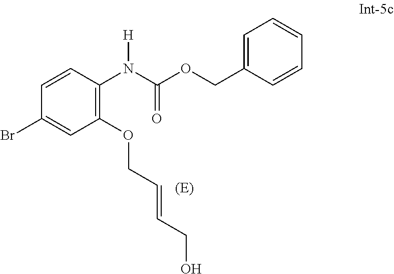 [SMILES: O=C(Nc1ccc(Br)cc1OC/C=C/CO)OCc1ccccc1]

To a mixture of NaHCO₃ (12.21 g, 145.29 mmol) and Int-5b (7.5 g, 29.06 mmol) in THF (100 mL) was added carbobenzoxy chloride (5.45 g, 31.96 mmol). The mixture was stirred at 25° C. for 12 h. After the reaction was completed, the mixture was diluted with EtOAc (400 mL) and filtered. The organic layer was washed with brine (100 mL×2), dried with anhydrous sodium sulfate and concentrated in vacuo to give crude product, which was purified by silica-gel chromatography (elute with EtOAc:PE=0-50%) to give Int-5c (9.0 g, 78.9% yield) as a yellow solid. MS (ESI⁺) obsd. [(M+Na)⁺]: 413.8.

### Step 4: Benzyl N-[4-bromo-2-[[3-(hydroxymethyl) oxiran-2-yl]methoxy]phenyl]carbamate

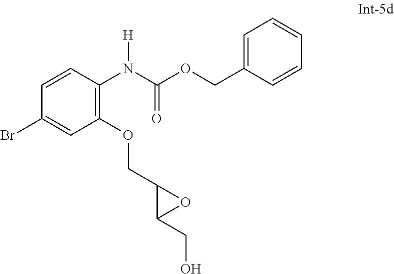 [SMILES: O=C(Nc1ccc(Br)cc1OCC1OC1CO)OCc1ccccc1]

To a solution of Int-5c (9.0 g, 22.95 mmol) in THF (50 mL) was added m-CPBA (14.88 g, 29.83 mmol). The mixture was stirred at 25° C. for 12 h. After the reaction was completed, the mixture was diluted with EA (150.0 mL) and washed with aq. Na₂SO₃ (50 mL×4) and aq.NaHCO₃ (50 mL×3). The combined organic layer was washed with brine, dried over anhydrous sodium sulfate, filtered and the filtrate was concentrated in vacuo to give crude Int-5d (9.0 g, 96.1% yield) as a yellow solid, which was used in next step directly without further purification. MS (ESI⁺) obsd. [(M+H)⁺]: 407.9.

### Step 5: Benzyl N-[4-bromo-2-[[3-[[tert-butyl(dimethyl)silyl]oxymethyl]oxiran-2-yl]methoxy]phenyl]carbamate (Int-9-6)

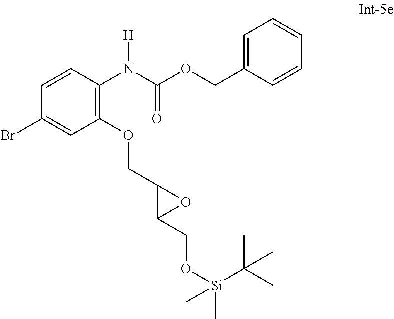 [SMILES: CC(C)(C)[Si](C)(C)OCC1OC1COc1cc(Br)ccc1NC(=O)OCc1ccccc1]

To a solution of Int-5d (9.0 g, 22.05 mmol), imidazole (3.01 g, 44.09 mmol) and 4-dimethylaminopyridine (0.54 g, 4.41 mmol) in DMF (25 mL) was added tert-butyldimethylchlorosilane (3.66 g, 24.25 mmol) at 0° C. The mixture was stirred at 0° C. for 2 h under nitrogen. After the reaction was completed, the mixture was diluted with water (200 mL) and extracted with EtOAc (250 mL×2). The combined organic layer was washed with aq. CaCl2 (100 mL×3), brine (50 mL×2), dried with anhydrous sodium sulfate, then filtered. The filtrate was concentrated in vacuo to give crude product, which was purified by silica-gel chromatography (elute with EtOAc:PE=0-30%) to give Int-5e (9.0 g, 78.1% yield) as yellow solid. MS (ESI+) obsd. (M+H)+]: 524.0.

### Step 6: cis-7-Bromo-3-[[tert-butyl(dimethyl)silyl]oxymethyl]-3a,4-dihydro-3H-oxazolo[4,3-c][1,4]benzoxazin-1-one

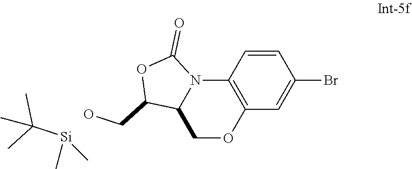

To a solution of Int-5e (8.5 g, 16.27 mmol) in THF (25 mL) were added LiHMDS (24.4 mL, 24.4 mmol) under nitrogen at −78° C. The mixture was slowly warmed up to 25° C. and stirred for 12 h under nitrogen. The mixture was poured into aq.NH₄Cl (100.0 mL) and extracted with EtOAc (100 mL×3). The organic layer was washed with brine (100 mL×2), dried with anhydrous Na₂SO₄ and filtered. The filtrate was concentrated in vacuo to give crude Int-5f (6.0 g, 89.0% yield) as yellow solid, which was used in next step directly without purification. MS (ESI⁺) obsd. (M+H)⁺]: 413.8.

### Step 7: cis-7-Bromo-3-(hydroxymethyl)-3a,4-dihydro-3H-oxazolo[4,3-c][1,4]benzoxazin-1-one

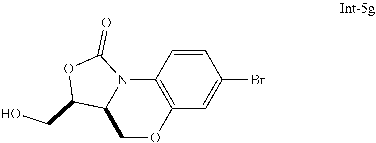 [SMILES: O=C1O[C@H](CO)[C@@H]2COc3cc(Br)ccc3N12]

To a solid of Int-5f (6.0 g, 14.48 mmol) was added TBAF (15.0 mL, 15 mmol) under nitrogen at −78° C. The mixture was slowly warmed up to 25° C. and stirred for 1 h under nitrogen. After the reaction was completed, the mixture was poured into aq.NH₄Cl (200 mL) and extracted with EtOAc (100 mL×3). The organic layer was washed with brine (100 mL×2), dried with anhydrous sodium sulfate and filtered. The filtrate was concentrated in vacuo to give crude product, which was purified by silica-gel chromatography (elute with EtOAc:PE=0-20%) to give Int-5g (4.0 g, 92.1% yield) as a white solid. MS (ESI⁺) obsd. [(M+H)⁺]: 301.7.

### Step 8: cis-3-(Aminomethyl)-7-bromo-3a,4-dihydro-3H-oxazolo[4,3-c][1,4]benzoxazin-1-one

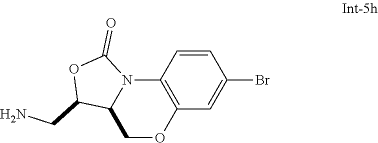 [SMILES: NC[C@H]1OC(=O)N2c3ccc(Br)cc3OC[C@@H]12]

To a solution of trifluoromethanesulfonic anhydride (1.03 g, 3.67 mmol) in DCM (8.89 mL) was added DIPEA (859.68 mg, 6.66 mmol) and Int-5g (1.0 g, 3.33 mmol) at −20° C. The mixture was stirred at −20° C. for 0.3 h. Then the mixture was added ammonia in THF (33.32 mL, 33.32 mmol) and stirred for 1 h for 45° C. After the reaction was completed, the mixture was diluted with EtOAc (200 mL), washed with brine (50 mL×2), dried over anhydrous sodium sulfate and filtered. The filtrate was concentrated in vacuo to give crude Int-5h (990 mg, 99.3% yield) as a light yellow oil, which was used in next step directly without purification. MS (ESI⁺) obsd. [(M+H)⁺]: 300.8.

### Step 9: tert-Butyl N-[[cis-7-bromo-1-oxo-3a,4-dihydro-3H-oxazolo[4,3-c][1,4]benzoxazin-3-yl]methyl]carbamate

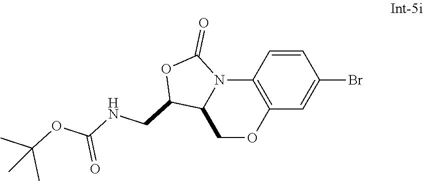 [SMILES: CC(C)(C)OC(=O)NC[C@H]1OC(=O)N2c3ccc(Br)cc3OC[C@@H]12]

To a solution of Int-5h (990.0 mg, 3.31 mmol) and DIPEA (500 mg, 3.87 mmol) in DCM (10 mL) was added di-t-butyldicarbonate (758.46 mg, 3.48 mmol) dropwise at 25° C. The mixture was stirred for 1 h. After the reaction was completed, the mixture was diluted with EtOAc (200 mL), washed with brine (50 mL×2), dried with anhydrous sodium sulfate and filtered. The filtrate was concentrated in vacuo to give a crude product, which was triturated in MTBE (30.0 mL) to give Int-5i (1.1 g, 83.2% yield) as a light yellow solid. MS (ESI⁺) obsd. [(M−100+H)⁺]: 298.8.

### Step 10: tert-Butyl N-[[cis-7-benzylsulfanyl-1-oxo-3a,4-dihydro-3H-oxazolo[4,3-c][1,4]benzoxazin-3-yl]methyl]carbamate

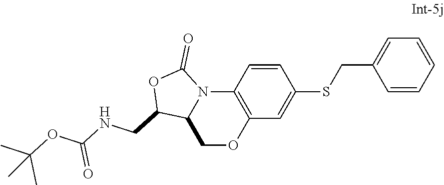 [SMILES: CC(C)(C)OC(=O)NC[C@H]1OC(=O)N2c3ccc(SCc4ccccc4)cc3OC[C@@H]12]

To a solution of Int-5i (1.1 g, 2.76 mmol) in DMF (15 mL) was added Et₃N (1.0 mL, 11.02 mmol), benzyl mercaptan (1.03 g, 8.27 mmol), tris(dibenzylideneacetone) dipalladium (0) (252.1 mg, 0.280 mmol) and 4,5-bis(diphenylphosphino)-9, 9-dimethylxanthene (318.61 mg, 0.55 mmol) under nitrogen. The mixture was stirred at 100° C. for 15 h under N₂. After the reaction was completed, the reaction mixture was poured into water (100 mL) and extracted with EtOAc (100 mL×3). The combined organic layer was washed with aq.CaCl₂ (100 mL×3) and brine (50 mL×3), dried over Na₂SO₄ and filtered. The filtrate was concentrated in vacuo to give a residue which was purified by silica-gel chromatography (elute with EtOAc:PE=0-30%) to give Int-5j (1.1 g, 90.2% yield) as a yellow oil. MS (ESI⁺) obsd. [(M−56+H)⁺]: 386.9.

### Step 11: tert-Butyl N-[[cis-7-chlorosulfonyl-1-oxo-3a,4-dihydro-3H-oxazolo[4,3-c][1,4]benzoxazin-3-yl]methyl]carbamate

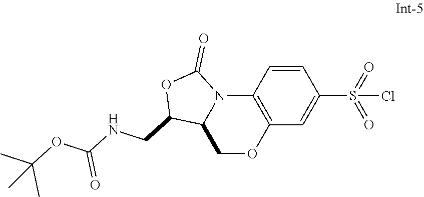 [SMILES: CC(C)(C)OC(=O)NC[C@H]1OC(=O)N2c3ccc(S(=O)(=O)Cl)cc3OC[C@@H]12]

To a mixture of Int-5j (1.1 g, 2.49 mmol) in THF (10 mL) and acetic acid (1 mL) was added 1,3-dichloro-5, 5-dimethylimidazolidine-2,4-dione (0.98 g, 4.97 mmol) and water (0.5 mL, 27.75 mmol) at 0° C. Then the mixture was stirred at 25° C. for 1 h. After the reaction was completed, the reaction was quenched with water (50 mL) and extracted with ethyl acetate (100 mL×3). The combined organic layer was dried over anhydrous Na₂SO₄ and filtered. The filtrate was concentrated in vacuo to give crude Int-5 (1.0 g, 96.1% yield) as light yellow oil. MS (ESI⁺) obsd. [(M−56+H)⁺]: 318.8.

### Intermediate 6: tert-Butyl N-[[cis-1-oxo-7-piperazin-1-ylsulfonyl-3a,4-dihydro-3H-oxazolo[4,3-c][1,4]benzoxazin-3-yl]methyl]carbamate

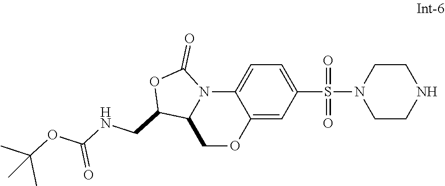 [SMILES: CC(C)(C)OC(=O)NC[C@H]1OC(=O)N2c3ccc(S(=O)(=O)N4CCNCC4)cc3OC[C@@H]12]

To a solution of piperazine (2.06 g, 23.87 mmol) in DCM (20 mL) were added Int-5 (1.0 g, 2.39 mmol) at 25° C. and the mixture was stirred for 2 h. After the reaction was completed, the mixture was poured into water (50 mL) and extracted with EtOAc (100 mL×3). The combined organic layer was dried over anhydrous Na₂SO₄, filtered and the filtrate was concentrated in vacuo to give crude product, which was purified by prep-HPLC (FA as additive) to give Int-6 (950 mg, 84.9% yield) as a white solid. MS (ESI⁺) obsd. [(M+H)⁺]: 469.1.

### Intermediate 7: tert-Butyl N-[[cis-7-chlorosulfonyl-1-oxo-3a,4-dihydro-3H-oxazolo[4,3-c][1,4]benzoxazin-3-yl]methyl]carbamate

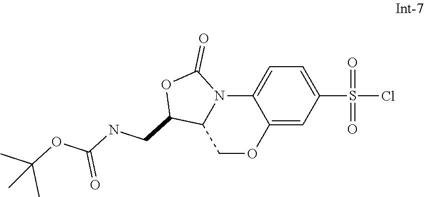 [SMILES: CC(C)(C)OC(=O)NC[C@H]1OC(=O)N2c3ccc(S(=O)(=O)Cl)cc3OC[C@H]12]

The title compound was prepared in analogy to the preparation of Int-5 by using (Z)-but-2-ene-1,4-dio instead of (E)-but-2-ene-1,4-diol in step 1. Int-7 was obtained as a yellow oil (200 mg, 100%). MS (ESI⁺) obsd. [(M+Na)⁺]: 441.0.

### Intermediate 8: tert-Butyl N-[[trans-1-oxo-7-piperazin-1-ylsulfonyl-3a,4-dihydro-3H-oxazolo[4,3-c][1,4]benzoxazin-3-yl]methyl]carbamate

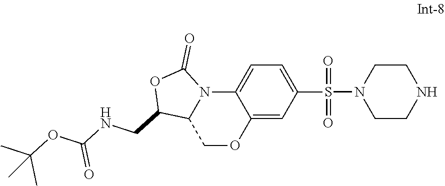 [SMILES: CC(C)(C)OC(=O)NC[C@H]1OC(=O)N2c3ccc(S(=O)(=O)N4CCNCC4)cc3OC[C@H]12]

The title compound was prepared in analogy to the preparation of Int-6 by using Int-7 instead of Int-5. Int-8 (221.6 mg, 66.04) as a yellow solid was obtained. MS (ESI⁺) obsd. [(M+H)⁺]: 469.1.

### Intermediate 9: Benzyl N-[[cis-1-oxo-7-piperazin-1-ylsulfonyl-3a,4-dihydro-3H-oxazolo[4,3-c][1,4]benzoxazin-3-yl]methyl]carbamate

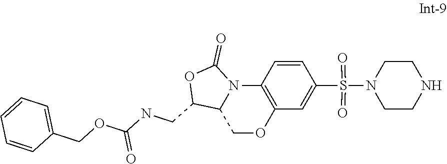 [SMILES: O=C(NC[C@@H]1OC(=O)N2c3ccc(S(=O)(=O)N4CCNCC4)cc3OC[C@H]12)OCc1ccccc1]

### Step 1: tert-Butyl 4-[[cis-3-(aminomethyl)-1-oxo-3a,4-dihydro-3H-oxazolo[4,3-c][1,4]benzoxazin-7-yl]sulfonyl]piperazine-1-carboxylate

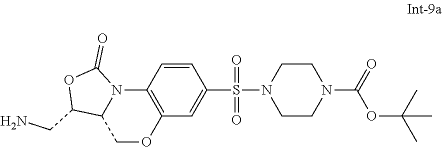 [SMILES: CC(C)(C)OC(=O)N1CCN(S(=O)(=O)c2ccc3c(c2)OC[C@@H]2[C@H](CN)OC(=O)N32)CC1]

To a solution of tert-butyl 4-[[cis-(3R,3aR)-3-(hydroxymethyl)-1-oxo-3a,4-dihydro-3H-oxazolo[4,3-c][1,4]benzoxazin-7-yl]sulfonyl]piperazine-1-carboxylate (1.0 g, 2.13 mmol) and DIPEA (1.85 mL, 10.65 mmol) in DCM (20 mL) was added Tf₂O (3.0 g, 10.65 mmol) at −20° C. The mixture was stirred at −20° C. for 1 h. After the reaction was completed, the mixture was quenched with NH₃ in THF (50.0 mL) solution at −78° C. The mixture was slowly warmed up to room temperature and concentrated in vacuo to give crude product, which was purified by prep-HPLC (TFA as additive) to give Int-9a (600 mg, 60.1% yield) as a brown solid. MS (ESI⁺) obsd. [(M+H)⁺]: 469.2.

### Step2: tert-Butyl 4-[[cis-3-(aminomethyl)-1-oxo-3a,4-dihydro-3H-oxazolo[4,3-c][1,4]benzoxazin-7-yl]sulfonyl]piperazine-1-carboxylate

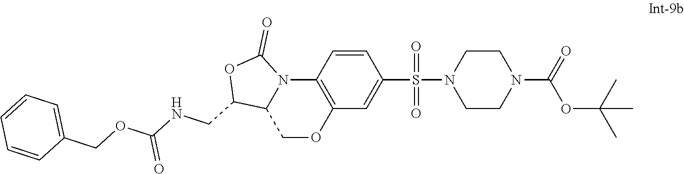 [SMILES: CC(C)(C)OC(=O)N1CCN(S(=O)(=O)c2ccc3c(c2)OC[C@@H]2[C@H](CNC(=O)OCc4ccccc4)OC(=O)N32)CC1]

To a solution of Int-9a (270.0 mg, 0.58 mmol) and triethylamine (0.24 mL, 1.73 mmol) in DCM (2 mL) was added benzyl succinimido carbonate (172.4 mg, 0.69 mmol) dropwise at 25° C. and the mixture was stirred at rt for 1 h. After the reaction was completed, the mixture was diluted with water (200 mL), extracted with EtOAc (200 mL×2) and washed with aq.NaHCO₃ (400 mL). The organic layer was dried over anhydrous sodium sulfate, filtered and the filtrate was concentrated in vacuo to give a residue, which was purified by silica-gel chromatography (eluted with EtOAc:PE=0-50%) to afford Int-9b (270.0 mg, 77.2% yield) as a light yellow solid. MS (ESI⁺) obsd. [(M+H)⁺]: 603.2.

### Step3: Benzyl N-[[cis-1-oxo-7-piperazin-1-ylsulfonyl-3a,4-dihydro-3H-oxazolo[4,3-c][1,4]benzoxazin-3-yl]methyl]carbamate

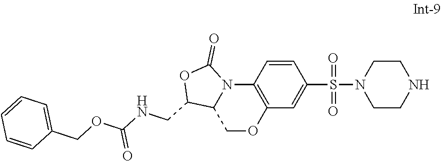 [SMILES: O=C(NC[C@@H]1OC(=O)N2c3ccc(S(=O)(=O)N4CCNCC4)cc3OC[C@H]12)OCc1ccccc1]

The mixture of Int-9b (200.0 mg, 0.330 mmol) in DCM (1 mL) and trifluoroacetic acid (1.0 mL) was stirred at 25° C. for 1 h. After the reaction was completed, the mixture was concentrated in vacuo to give crude Int-9 (200 mg, 100% yield) as a yellow solid without further purification. MS (ESI⁺) obsd. [(M+H)⁺]: 503.2.

### Intermediate 10: Benzyl N-[[trans-1-oxo-7-piperazin-1-ylsulfonyl-3a,4-dihydro-3H-oxazolo[4,3-c][1,4]benzoxazin-3-yl]methyl]carbamate

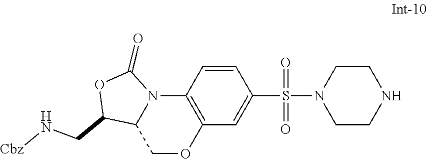 [SMILES: O=C(NC[C@H]1OC(=O)N2c3ccc(S(=O)(=O)N4CCNCC4)cc3OC[C@H]12)OCc1ccccc1]

The title compound was prepared in analogy to the preparation of Int-9 by using tert-butyl 4-[[trans-3-(hydroxymethyl)-1-oxo-3a,4-dihydro-3H-oxazolo[4,3-c][1,4]benzoxazin-7-yl]sulfonyl]piperazine-1-carboxylate instead of tert-butyl 4-[[cis-3-(hydroxymethyl)-1-oxo-3a,4-dihydro-3H-oxazolo[4,3-c][1,4]benzoxazin-7-yl]sulfonyl]piperazine-1-carboxylate in step 1. Int-10 was obtained as a yellow solid (1000 mg, 76.4% yield). MS (ESI⁺) obsd. [(M+H)⁺]: 503.1.

### Intermediate 11:1-Oxo-2,3,3a,4-tetrahydropyrrolo[2,1-c][1,4]benzoxazine-7-sulfonyl chloride

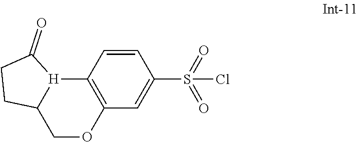

### Step 1: 1-(4-Bromo-2-fluoro-phenyl)-5-(hydroxymethyl)pyrrolidin-2-one

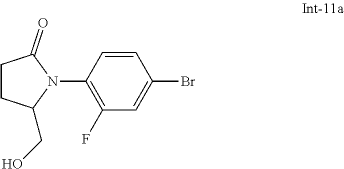 [SMILES: O=C1CCC(CO)N1c1ccc(Br)cc1F]

A mixture of 5-(hydroxymethyl)pyrrolidin-2-one (1147.89 mg, 9.97 mmol), 4-bromo-2-fluoro-1-iodo-benzene (3.0 g, 9.97 mmol), potassium phosphate (4.13 g, 29.91 mmol), copper (I) iodide (0.34 mL, 9.97 mmol) and N,N′-dimethylethylenediamine (878.89 mg, 9.97 mmol)) in 1,4-dioxane (60 mL) was stirred at 100° C. for 1 h under N₂ protection. After the reaction was completed, the mixture was filtered and the filtrate was concentrated in vacuo to afford a crude, which was purified by silica-gel chromatography (elute with EtOAc:PE=0-50%) to give Int-11a (375 mg, 13.05% yield) as a colorless oil. MS (ESI⁺) obsd. [(M+H)⁺]: 288.0.

### Step 2:7-Bromo-2,3,3a,4-tetrahydropyrrolo[2,1-c][1,4]benzoxazin-1-one

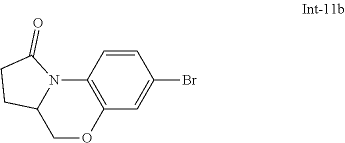 [SMILES: O=C1CCC2COc3cc(Br)ccc3N12]

A solution of Int-11a (365.0 mg, 1.27 mmol) and cesium carbonate (1238.0 mg, 3.8 mmol) in DMF (2 mL) was stirred at 100° C. for 16 h. After the reaction was completed, the mixture was quenched with water (20 mL) and extracted with EtOAc (20 mL×3). The combined organic layer was washed with brine (50 mL), dried over anhydrous Na₂SO₄ and filtered. The filtrate was concentrated in vacuo to afford a residue, which was purified by prep-HPLC (TFA as additive) to give Int-11b (160 mg, 47.1% yield) as a white solid. MS (ESI⁺) obsd. [(M+H)⁺]: 270.0.

### Step 3: 7-Benzylsulfanyl-2,3,3a,4-tetrahydropyrrolo[2,1-c][1,4]benzoxazin-1-one

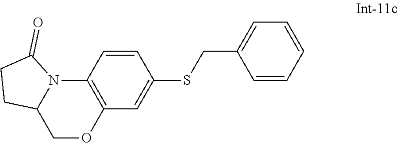 [SMILES: O=C1CCC2COc3cc(SCc4ccccc4)ccc3N12]

A mixture of benzyl mercaptan (120.0 mg, 0.970 mmol), Int-11b (200.0 mg, 0.750 mmol), tris(dibenzylideneacetone)dipalladium(0) (204.77 mg, 0.220 mmol), 4,5-bis(diphenylphosphino)-9,9-dimethylxanthene (86.26 mg, 0.150 mmol) and DIPEA (0.26 mL, 1.49 mmol) in 1,4-dioxane (3 mL) was stirred at 110° C. for 16 h under N₂ protection. After the reaction was completed, the mixture was diluted with DCM (20 mL), filtered and the filtrate was concentrated in vacuo to afford a crude, which was purified by prep-HPLC (TFA as additive) to give Int-11c (56 mg, 24.11% yield) as a yellow oil. MS (ESI⁺) obsd. [(M+H)⁺]: 312.2.

### Step 4: 1-Oxo-2,3,3a,4-tetrahydropyrrolo[2,1-c][1,4]benzoxazine-7-sulfonyl chloride

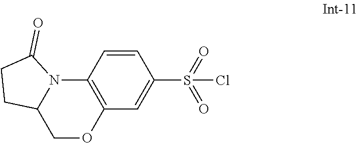 [SMILES: O=C1CCC2COc3cc(S(=O)(=O)Cl)ccc3N12]

To a solution of Int-11c (56.0 mg, 0.180 mmol) in THF (1 mL), acetic acid (0.100 mL) and water (0.100 mL) was added 1,3-dichloro-5,5-dimethylhydantoin (53.15 mg, 0.270 mmol) at 0° C. After addition, the reaction was allowed to warmed up to 25° C. and was stirred for 1 h. After the reaction was completed, the mixture was filtered and the filtrate was concentrated in vacuo to afford crude Int-11 (50 mg, 96.63% yield) as a yellow oil, which was used without further purification. MS (ESI⁺) obsd. [(M+H)⁺]: 288.7.

### Intermediate 12:7-Piperazin-1-ylsulfonyl-2,3,3a,4-tetrahydropyrrolo[2,1-c][1,4]benzoxazin-1-one

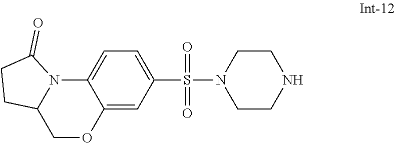 [SMILES: O=C1CCC2COc3cc(S(=O)(=O)N4CCNCC4)ccc3N12]

### Step 1. tert-Butyl 4-(4-bromo-3-fluoro-phenyl)sulfonylpiperazine-1-carboxylate

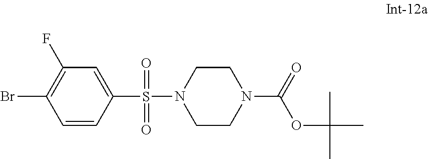 [SMILES: CC(C)(C)OC(=O)N1CCN(S(=O)(=O)c2ccc(Br)c(F)c2)CC1]

A mixture of 4-bromo-3-fluorobenzenesulfonyl chloride (3.46 g, 12.7 mmol), tert-butyl piperazine-1-carboxylate (2.36 g, 12.7 mmol) and triethylamine (3.84 g, 5.29 mL, 38 mmol) was stirred at room temperature overnight. After the reaction was completed, the mixture was diluted with EtOAc (20 mL) and washed with water (10 mL×2). The organic phase was dried over anhydrous sodium sulfate and filtered. The filtrate was concentrated in vacuo to give crude product, which was purified by silica-gel chromatography (elute with EtOAc:PE=0-20%) to give Int-12a (4.6 g, 86%). MS (ESI⁺) obsd. [(M+H)⁺]: 423.2.

### Step 2. tert-Butyl 4-[3-fluoro-4-[2-(hydroxymethyl)-5-oxo-pyrrolidin-1-yl]phenyl]sulfonylpiperazine-1-carboxylate

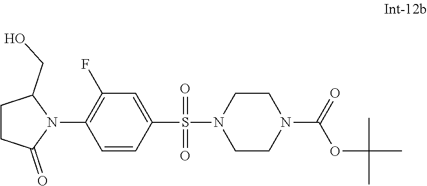 [SMILES: CC(C)(C)OC(=O)N1CCN(S(=O)(=O)c2ccc(N3C(=O)CCC3CO)c(F)c2)CC1]

A degassed mixture of Int-12a (0.50 g, 1.18 mmol), 5-(hydroxymethyl)pyrrolidin-2-one (136 mg, 1.18 mmol), N,N′-dimethylethylenediamine (20.8 mg, 236 μmol), CuI (45 mg, 236 μmol) and K₃PO₄ (751 mg, 3.54 mmol) in DMF (1.5 mL) was heated at 80° C. for 2 h. After the reaction was completed, the mixture was poured into water, extracted with DCM (10 mL×3). The organic layer was concentrated under vacuum to give crude, which was purified by silica-gel chromatography (elute with EtOAc:PE=0-100%) to give Int-12b (220 mg, 41.0% yield). MS (ESI⁺) obsd. [(M+H)⁺]: 458.2.

### Step 3. tert-Butyl 4-[(1-oxo-2,3,3a,4-tetrahydropyrrolo[2,1-c][1,4]benzoxazin-7-yl)sulfonyl]piperazine-1-carboxylate

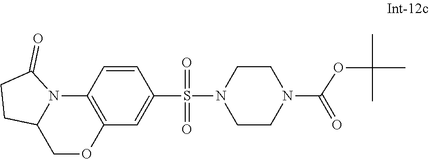 [SMILES: CC(C)(C)OC(=O)N1CCN(S(=O)(=O)c2ccc3c(c2)OCC2CCC(=O)N32)CC1]

A degassed mixture of Int-12b (0.04 g, 87.4 μmol) and cesium carbonate (42.7 mg, 131 μmol) in DMF (1 mL) was heated at 100° C. overnight. After the reaction was completed, the mixture was poured into water, extracted with DCM (10 mL×3). The organic layer was concentrated in vacuo to give crude product, which was purified by silica-gel chromatography (elute with EtOAc:PE=0-50%) to give Int-12c (33 mg, 86%). MS (ESI⁺) obsd. [(M+H)⁺]: 438.2.

### Step 4. 7-Piperazin-1-ylsulfonyl-2,3,3a,4-tetrahydropyrrolo[2,1-c][1,4]benzoxazin-1-one

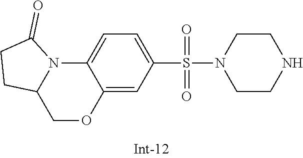 [SMILES: O=C1CCC2COc3cc(S(=O)(=O)N4CCNCC4)ccc3N12]

A mixture of Int-12c (0.120 g, 274 μmol) and 2,2,2-trifluoroacetic acid (767 mg, 6.73 mmol) in DCM (1 mL) was stirred at 25° C. for 1 h. After the reaction was completed, the mixture was concentrated in vacuo to give crude Int-12 (92.5 mg, 100%). MS (ESI⁺) obsd. [(M+H)⁺]: 338.2.

### Intermediate 13: tert-Butyl N-(1-oxo-7-piperazin-1-ylsulfonyl-2,3,3a,4-tetrahydropyrrolo[2,1-c][1,4]benzoxazin-3-yl)carbamate

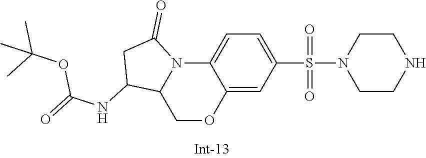 [SMILES: CC(C)(C)OC(=O)NC1CC(=O)N2c3ccc(S(=O)(=O)N4CCNCC4)cc3OCC12]

### Step 1: tert-Butyl N-[2-[(5-bromo-2-iodo-phenoxy)methyl]-5-oxo-pyrrolidin-3-yl]carbamate

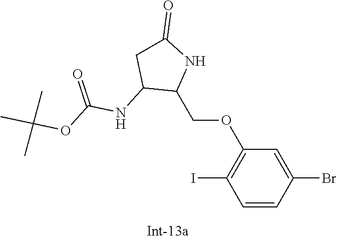 [SMILES: CC(C)(C)OC(=O)NC1CC(=O)NC1COc1cc(Br)ccc1I]

A mixture of tri-N-butylphosphine (764.43 mg, 3.78 mmol), 5-bromo-2-iodo-phenol (752.9 mg, 2.52 mmol), tert-butyl N-[2-(hydroxymethyl)-5-oxo-pyrrolidin-3-yl]carbamate (580.0 mg, 2.52 mmol) and N,N,N′,N′-tetramethylazodicarboxamide (650.59 mg, 3.78 mmol) in THF (20.0 mL) under nitrogen was stirred at 60° C. for 12 h. After the reaction was completed, the mixture was diluted with EtOAc (200 mL) and washed with aq. HCl (0.5 N, 50 mL×2), brine (50 mL×2), dried over anhydrous Na₂SO₄ and then filtered. The filtrate was concentrated in vacuo to give a residue, which was recrystallized with PE/MTBE (v/v=10:1, 50 mL) to afford Int-13a (1.1 g, 85.4% yield) as a white solid. MS (ESI⁺) obsd. [(M+H)⁺]: 511.2.

### Step 2: tert-Butyl N-(7-bromo-1-oxo-2,3,3a,4-tetrahydropyrrolo[2,1-c][1,4]benzoxazin-3-yl)carbamate

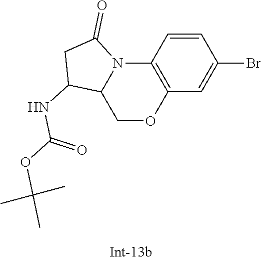 [SMILES: CC(C)(C)OC(=O)NC1CC(=O)N2c3ccc(Br)cc3OCC12]

A degassed mixture of copper iodide (74.52 mg, 0.390 mmol), N,N′-dimethylethane-1,2-diamine (51.71 mg, 0.59 mmol), Int-13a (1.0 g, 1.96 mmol) and potassium carbonate (1.35 g, 9.78 mmol) in NMP (30 mL) was stirred at 50° C. for 2 h. After the reaction was completed, the mixture was diluted with EtOAc (300 mL), washed with aq. NH₃·H₂O (50 mL×2, 2% in H₂O), aq. CaCl₂ (50 mL×3) and brine (100 mL×2), the organic layer was dried over anhydrous Na₂SO₄ and filtered. The filtrate was concentrated in vacuo to give crude product, which was purified by prep-HPLC (TFA as additive) to give Int-13b (749 mg, 99.9% yield) as a white solid. MS (ESI⁺) obsd. [(M+H)⁺]: 383.2.

### Step 3: tert-butyl N-(7-benzylsulfanyl-1-oxo-2,3,3a,4-tetrahydropyrrolo[2,1-c][1,4]benzoxazin-3-yl)carbamate

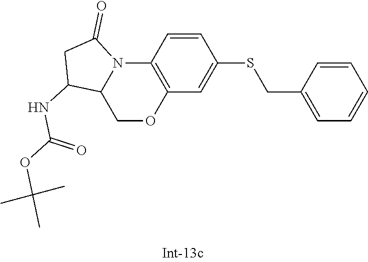 [SMILES: CC(C)(C)OC(=O)NC1CC(=O)N2c3ccc(SCc4ccccc4)cc3OCC12]

The title compound was prepared in analogy to the preparation of Int-11c, replacing Int-11b with Int-13b. Int-13c (950 mg) was obtained as a yellow solid. MS (ESI⁺) obsd. [(M+H)⁺]: 427.0.

### Step 4: tert-Butyl N-(7-chlorosulfonyl-1-oxo-2,3,3a,4-tetrahydropyrrolo[2,1-c][1,4]benzoxazin-3-yl)carbamate

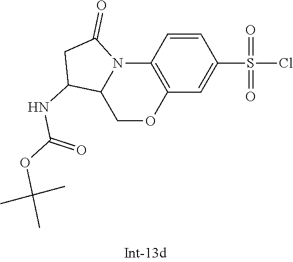 [SMILES: CC(C)(C)OC(=O)NC1CC(=O)N2c3ccc(S(=O)(=O)Cl)cc3OCC12]

The title compound was prepared in analogy to the preparation of Int-11, replacing Int-11c with Int-13c in step 4. Int-13d (850 mg) was obtained as a white solid. MS (ESI⁺) obsd. [(M+H)⁺]: 403.1.

### Step 5: tert-Butyl N-(1-oxo-7-piperazin-1-ylsulfonyl-2,3,3a,4-tetrahydropyrrolo[2,1-c][1,4]benzoxazin-3-yl)carbamate

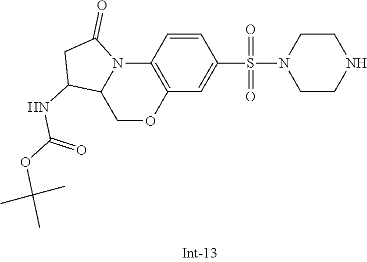 [SMILES: CC(C)(C)OC(=O)NC1CC(=O)N2c3ccc(S(=O)(=O)N4CCNCC4)cc3OCC12]

To a solution of piperazine (3.64 g, 42.2 mmol) in THF (40 mL) and water (5 mL) were added Int-12 (0.85 g, 2.11 mmol) in one portion. The mixture was stirred at 25° C. for 1 h. After the reaction was completed, the mixture was poured into water (50 mL) and extracted with ethyl acetate (100 mL×3). The combined organic layer was concentrated in vacuo to give a residue. which was purified by prep-HPLC (TFA as additive to give Int-13 (840 mg, 87.9% yield) as a white solid. MS (ESI⁺) obsd. [(M+H)⁺]: 453.3.

### Intermediate 14:1-[6-Chloro-4-(trifluoromethyl)-2-pyridyl]piperazine

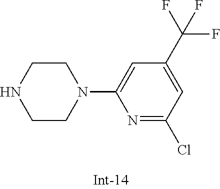 [SMILES: FC(F)(F)c1cc(Cl)nc(N2CCNCC2)c1]

A mixture of piperazine (49.85 g, 578.73 mmol) and 2,6-dichloro-4-(trifluoromethyl)pyridine (25.0 g, 115.75 mmol) in NMP (50 mL) was stirred at 60° C. for 1 h. After the reaction was completed, 0 the mixture was diluted with water (300 mL) and extracted with EtOAc (200 mL×3). The combined organic layer were washed with aq. CaCl2 (200 mL×3) and brine (300 mL×3), dried over anhydrous Na₂SO₄ and filtered. The filtrate was concentrated in vacuo to give Int-14 (28 g, 91.0% yield) as a white solid, which was used in next step without purification. MS (ESI⁺) obsd. [(M+H)⁺]: 266.1.

### Intermediate 15:4-Methyl-2-piperazin-1-yl-6-(trifluoromethyl)pyrimidine

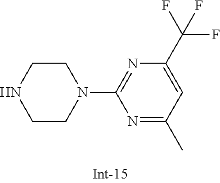 [SMILES: Cc1cc(C(F)(F)F)nc(N2CCNCC2)n1]

The title compound was prepared in analogy to the preparation of compound int-14 by using 2-chloro-4-methyl-6-(trifluoromethyl)pyrimidine instead of 2,6-dichloro-4-(trifluoromethyl)pyridine in step 1. Int-15 (400 mg, 1.62 mmol, 45.4% yield) as a yellow solid was obtained. MS (ESI⁺) obsd. [(M+H)⁺]: 247.1.

### Intermediate 16:1-(6-Chloro-4-cyclopropylsulfonyl-2-pyridyl) piperazine

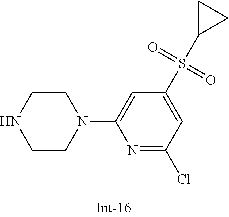 [SMILES: O=S(=O)(c1cc(Cl)nc(N2CCNCC2)c1)C1CC1]

### Step 1: 2,6-Dichloro-4-(cyclopropylthio)pyridine

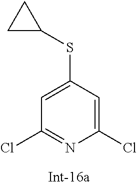 [SMILES: Clc1cc(SC2CC2)cc(Cl)n1]

A mixture of 4-bromo-2,6-dichloro-pyridine (100.0 mg, 0.440 mmol), cyclopropanethiol (32.68 mg, 0.440 mmol) and K₂CO₃ (0.25 mL, 1.32 mmol) in DMF (2 mL) was heated to 80° C. for 4 h. After the reaction was completed, the mixture was concentrated in vacuo to a residue, which was purified by prep-HPLC (TFA as additive) to give Int-16a (100 mg, 82.46% yield) as yellow oil. MS (ESI⁺) obsd. [(M+H)⁺]: 220.2.

### Step 2: 2,6-Dichloro-4-cyclopropylsulfonyl-pyridine

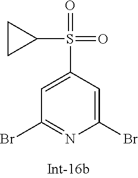 [SMILES: O=S(=O)(c1cc(Br)nc(Br)c1)C1CC1]

To a solution of Int-16a (80.0 mg, 0.360 mmol) in DCM (4 mL) were added m-CPBA (0.15 mL, 1.09 mmol) in one portion. The mixture was stirred at 20° C. for 2 h. After the reaction was completed, the mixture was concentrated to give a residue, which was purified by prep-HPLC (TFA as additive) to give Int-16b (60 mg, 765.48% yield) as a yellow solid. MS (ESI⁺) obsd. [(M+H)⁺]: 252.9.

### Step 3: 1-(6-chloro-4-(cyclopropylsulfonyl)pyridin-2-yl) piperazine

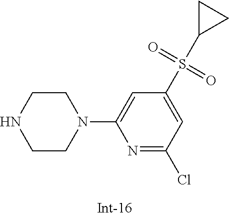 [SMILES: O=S(=O)(c1cc(Cl)nc(N2CCNCC2)c1)C1CC1]

A mixture of piperazine (49.2 mg, 0.570 mmol) and Int-16b (120.0 mg, 0.480 mmol) in DMSO (1 mL) was heated at 60° C. for 1 h. After the reaction was completed, the mixture was diluted with water (20 mL), extracted with EtOAc (20 mL×3). The combined organic layer were washed with brine (30 mL), dried over anhydrous Na₂SO₄ and filtered. The filtrate was concentrated in vacuo to give Int-16 (100 mg, 69.6% yield) as a yellow solid. MS (ESI⁺) obsd. [(M+H)⁺]: 302.1.

### Intermediate 17: tert-Butyl N-[4-[(2-chloro-6-piperazin-1-yl-4-pyridyl)sulfonyl]phenyl]carbamate

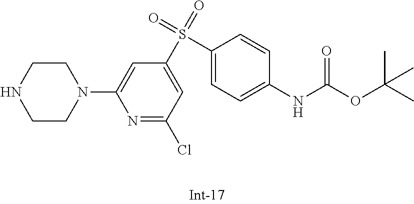 [SMILES: CC(C)(C)OC(=O)Nc1ccc(S(=O)(=O)c2cc(Cl)nc(N3CCNCC3)c2)cc1]

### Step 1: 4-[(2,6-Dichloro-4-pyridyl)sulfanyl]aniline

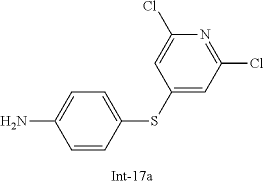 [SMILES: Nc1ccc(Sc2cc(Cl)nc(Cl)c2)cc1]

A mixture of 2,4,6-trichloropyridine (4.0 g, 21.93 mmol), potassium carbonate (9.09 g, 65.78 mmol) and 4-aminothiophenol (2.88 g, 23.02 mmol) in MeCN (30 mL) was stirred at 15° C. for 1 h. After the reaction was completed, the mixture was diluted with EtOAc (200.0 mL) and washed with aq. NH₄Cl (100 mL×2), brine (100 mL×3), dried over anhydrous Na₂SO₄ and filtered. The filtrate was concentrated in vacuo to give a residue, which was purified by silica-gel chromatography (elute with EtOAc:PE=0-30%) to give Int-17a (4.8 g, 80.7% yield) as a white solid. MS (ESI⁺) obsd. [(M+H)⁺]: 270.7.

### Step 2: tert-Butyl N-[4-[(2,6-dichloro-4-pyridyl)sulfanyl]phenyl]carbamate

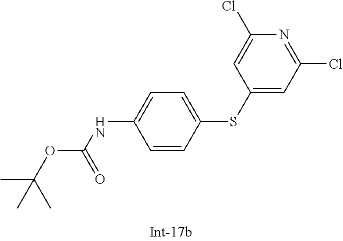 [SMILES: CC(C)(C)OC(=O)Nc1ccc(Sc2cc(Cl)nc(Cl)c2)cc1]

To a solution of Int-17a (3.0 g, 11.06 mmol) in THF (20 mL) were added Lithium bis(trimethylsilyl)amide (22.13 mL, 22.13 mmol) dropwise at 0° C. After addition, di-t-butyldicarbonate (2.41 g, 11.06 mmol) was added to the above mixture and the mixture was stirred at 15° C. for 1 h. After the reaction was completed, the mixture was diluted with EtOAc (400 mL), washed with aq. NH₄Cl (100 mL×2), brine (100 mL×3) and dried over anhydrous Na₂SO₄. The organic layer was concentrated in vacuo to give crude Int-17b (3.8 g, 92.5% yield) as a light yellow solid. MS (ESI⁺) obsd. [(M+H)⁺]: 370.8.

### Step 3: tert-Butyl N-[4-[(2,6-dichloro-4-pyridyl)sulfonyl]phenyl]carbamate

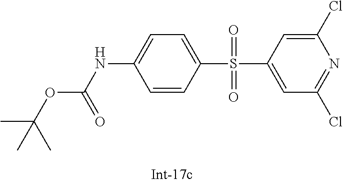 [SMILES: CC(C)(C)OC(=O)Nc1ccc(S(=O)(=O)c2cc(Cl)nc(Cl)c2)cc1]

To a solution of Int-17b (3.8 g, 10.23 mmol) in THF (40 mL) was added 3-chloroperoxybenzoic acid (5.19 g, 25.59 mmol) in one portion at rt. The reaction mixture was stirred at 45° C. for 2 h. After the reaction was completed, the mixture was diluted with EtOAc (400 mL) and washed with 10% Na₂SO₃ solution (100 mL×5). The organic layer was washed with brine (100 mL×2), dried over anhydrous Na₂SO₄, filtered and concentrated in vacuo to give a residue, which was purified by silica-gel chromatography (elute with EtOAc:PE=0-30%) to give Int-17c (3.5 g, 84.8% yield) as a white solid. MS (ESI⁺) obsd. [(M+H)⁺]: 402.8.

### Step 4: tert-Butyl N-[4-[(2-chloro-6-piperazin-1-yl-4-pyridyl)sulfonyl]phenyl]carbamate

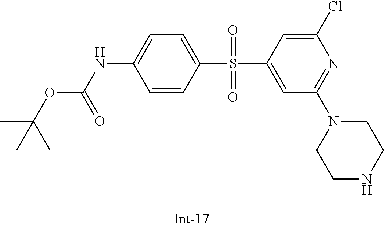 [SMILES: CC(C)(C)OC(=O)Nc1ccc(S(=O)(=O)c2cc(Cl)nc(N3CCNCC3)c2)cc1]

A mixture of piperazine (141.0 mg, 0.165 mmol) and Int-17c (200.0 mg, 0.50 mmol) in DMSO (3 mL) was stirred at 60° C. for 1 h. After the reaction was completed, the mixture was diluted with water (20 mL), extracted with EtOAc (20 mL×3). The combined organic layer was washed with brine (30 mL), dried over anhydrous Na₂SO₄ and filtered. The filtrate was concentrated in vacuo to give Int-17 (150 mg, 66.8% yield) as a yellow solid. MS (ESI⁺) obsd. [(M+H)⁺]: 453.1.

### Intermediate Int-18: 2,6-Dichloro-4-[1,4-dioxan-2-yl(difluoro)methyl]pyridine

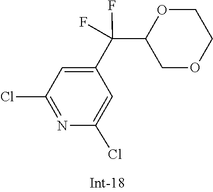 [SMILES: FC(F)(c1cc(Cl)nc(Cl)c1)C1COCCO1]

### Step 1: 1,4-Dioxane-2-carbaldehyde

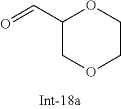

To a solution of DMSO (15.75 g, 315.1 mmol) in DCM (450 mL) was added (COCl)₂ (19.84 g, 157.6 mmol) dropwise at −60° C. and stirred for 1 h. And then, 1,4-Dioxan-2-ylmethanol (15.5 g, 131.3 mmol) was added to the mixture and stirred at −60° C. for another 2 h. Followed by adding Et₃N (39.83 g, 393.9 mmol) at −60° C. The mixture was warmed up to rt. The suspension was filtered and the filtrate was concentrated to give a crude mixture, which was distilled under vacuum pump and collected the 70° C. co-boiled to give Int-18a (21.5 g, purity: 50%, yield: 70.7%) as colorless liquid. ¹H NMR (400 MHz, CDCl₃) δ ppm 9.54-9.64 (m, 1H), 3.97-4.09 (m, 1H), 3.82-3.95 (m, 2H), 3.52-3.79 (m, 4H).

### Step 2: (2,6-Dichloro-4-pyridyl)-(1,4-dioxan-2-yl)methanol

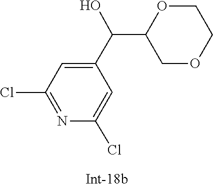 [SMILES: OC(c1cc(Cl)nc(Cl)c1)C1COCCO1]

To a mixture of Int-18a (21.46 g, 78.37 mmol) in THF (70 mL) was added isopropylmagnesium chloride-lithium chloride complex (64.92 mL, 84.4 mmol) at −50° C. and the mixture was stirred at 25° C. for 2 h. And then, a mixture of 1,4-dioxane-2-carbaldehyde (14.0 g, 60.28 mmol) in THF (10 mL) was added to the reaction mixture at −50° C. The mixture was warmed up to 25° C. and stirred for another 1 h. After the reaction was completed, the reaction mixture was poured into saturated NH₄Cl aq solution and extracted with ethyl acetate (300 mL×3). The combined organic layer was washed with brine, dried over anhydrous sodium sulfate, filtered and concentrated in vacuo to give a residue, which was purified by silica-gel chromatography (elute with EtOAc:PE=0-40%) to give Int-18b (8.3 g, 52.1% yield) as yellow oil. MS (ESI⁺) obsd. [(M+H)⁺]: 264.1.

### Step 3: (2,6-Dichloro-4-pyridyl)-(1,4-dioxan-2-yl)methanone

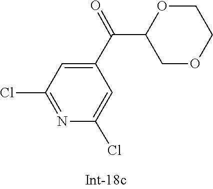 [SMILES: O=C(c1cc(Cl)nc(Cl)c1)C1COCCO1]

To a solution of Int-18b (6.4 g, 24.23 mmol) in DCM (60 mL) was added Dess-Martin periodinane (11.31 g, 26.66 mmol) at 0° C. The reaction was warmed up to 20° C. and stirred for 0.5 h. After the reaction was completed, the mixture was poured into aq. Na₂SO₃ (300 mL) and extracted with EtOAc (100 mL×3). The combined organic layer was concentrated in vacuo to give crude Int-18c (6 g, 94.5% yield) as light yellow solid.

### Step 4: 2,6-Dichloro-4-[1,4-dioxan-2-yl(difluoro)methyl]pyridine

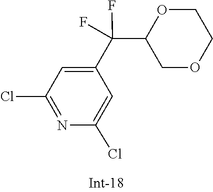 [SMILES: FC(F)(c1cc(Cl)nc(Cl)c1)C1COCCO1]

To a solution of Int-18c (7.0 g, 26.71 mmol) in DCM (150 mL) was added diethylaminosulfur trifluoride (140.0 mL, 1.06 mol) and the mixture was stirred at 20° C. for 12 h. After the reaction was completed, the reaction was poured onto ice-water (200 mL) and extracted with EtOAc (400 mL×3). The organic layer was concentrated under vacuum to give crude residue, which was purified by prep-HPLC (TFA as additive) to afford Int-18 (4 g, 52.72% yield) as white solid. MS (ESI⁺) obsd. [(M+H)⁺]: 284.1.

### Step 5: 2,6-Dichloro-4-[[(2R)-1,4-dioxan-2-yl]-difluoro-methyl]pyridine (Int-18-A) and 2,6-dichloro-4-[[(2S)-1,4-dioxan-2-yl]-difluoro-methyl]pyridine (Int-18-B)

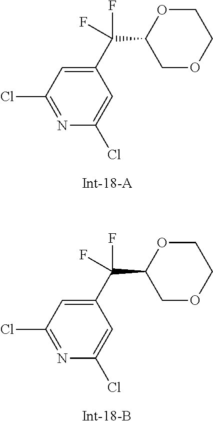 [SMILES: FC(F)(c1cc(Cl)nc(Cl)c1)[C@H]1COCCO1 ; FC(F)(c1cc(Cl)nc(Cl)c1)[C@@H]1COCCO1]

4.0 g of Int-18 was purified by SFC as the following condition to give Int-18-A (1.48 g, 99% ee, faster eluted) as a white solid and Int-18-B (1.50 g, 99% ee, slower eluted) as a white solid. Column: DAICEL CHIRALCEL OJ (250 mm×50 mm, 10 μm); Condition: 0.1% NH₃H₂O EtOH.

### Intermediate 19: 2,6-Dichloro-4-[difluoro(3-oxabicyclo[3.1.0]hexan-6-yl)methyl]pyridine

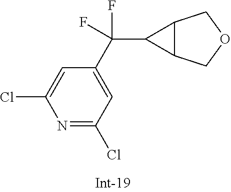 [SMILES: FC(F)(c1cc(Cl)nc(Cl)c1)C1C2COCC21]

The title compound was prepared in analogy to the preparation of Int-18 by using 3-oxabicyclo[3.1.0]hexan-6-ylmethanol instead of (1,4-dioxan-2-yl)methanol in Step 1. Int-19 (300 mg) was obtained as a white solid. MS (ESI⁺) obsd. [(M+H)⁺]: 280.1.

### Intermediate 20: 2,6-Dichloro-4-[difluoro-(2-methyl-4-pyridyl)methyl]pyridine

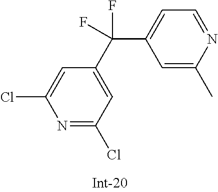 [SMILES: Cc1cc(C(F)(F)c2cc(Cl)nc(Cl)c2)ccn1]

The title compound was prepared in analogy to the preparation of Int-18 by using (2-methyl-4-pyridyl)methanol instead of (1,4-dioxan-2-yl)methanol in Step 1. Int-20 (108 mg) was obtained as a white solid. MS (ESI⁺) obsd. [(M+H)⁺]: 288.9.

### Intermediate 21: 2,6-Dichloro-4-[difluoro-(1-methyl-3,6-dihydro-2H-pyridin-4-yl)methyl]pyridine

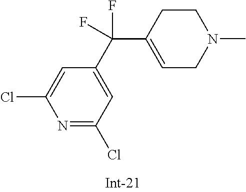 [SMILES: CN1CC=C(C(F)(F)c2cc(Cl)nc(Cl)c2)CC1]

### Step 1: (2,6-Dichloro-4-pyridyl)-(4-pyridyl)methanone

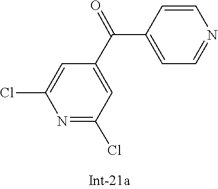 [SMILES: O=C(c1ccncc1)c1cc(Cl)nc(Cl)c1]

To a solution of n-butyllithium (2.5M, 15.19 mL, 36.46 mmol) in THF (50 mL) was added a solution of 2,6-dichloropyridine (5395.58 mg, 36.46 mmol) in THF (25 mL) at −78° C. under N₂. After addition, the mixture was stirred for another 30 minutes, and then a solution of methyl isonicotinate (5.0 mL, 36.46 mmol) in THF (25 mL) was added to above mixture and the mixture was stirred at −78° C. for another 6 h. After the reaction was completed, the reaction mixture was quenched with saturated aq. NH₄Cl (200 mL) and extracted with EtOAc (100 mL×3). The combined organic layer was concentrated in vacuo to give the crude product, which was purified by silica-gel chromatography (elute with EtOAc:PE=0-35%) to give Int-21a (2.6 g, 13.0% yield) as light brown oil. MS obsd. MS (ESI⁺) obsd. [(M+H)⁺]: 253.0.

### Step 2: 2,6-Dichloro-4-[difluoro(4-pyridyl)methyl]pyridine

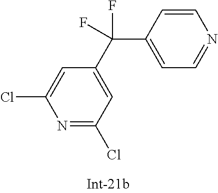 [SMILES: FC(F)(c1ccncc1)c1cc(Cl)nc(Cl)c1]

To a solution of Int-21a (2.0 g, 7.9 mmol) in DCM (5 mL) was added bis(2-methoxyethyl)aminosulfur trifluoride (10.0 mL, 7.9 mmol). The mixture was stirred at 25° C. for 18 h. After the reaction was completed, the reaction mixture was quenched with ice water (100 mL) and extracted with EtOAc (50 mL×3). The combined organic layer was concentrated in vacuo to give the crude product, which was purified by silica-gel chromatography (elute with EtOAc:PE=0-35%) to give Int-21b (800 mg, 29.4% yield) as light brown oil. MS (ESI⁺) obsd. [(M+H)⁺]: 275.1.

### Step 3: 2,6-Dichloro-4-[difluoro-(1-methylpyridin-1-ium-4-yl)methyl]pyridine; chloride

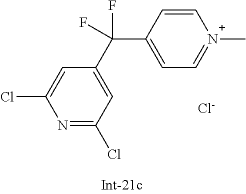 [SMILES: C[n+]1ccc(C(F)(F)c2cc(Cl)nc(Cl)c2)cc1 ; [Cl-]]

A mixture of Int-21b (300.0 mg, 1.09 mmol) and iodomethane (1.5 mL, 24.09 mmol) in EtOAc (10 mL) was stirred at 80° C. for 18 h. After the reaction was completed, the reaction mixture was concentrated in vacuo to give crude Int-21c (400 mg, 70.4% yield) as dark brown oil. MS obsd. MS (ESI⁺) obsd. [{(M−Cl)⁺]: 289.1.

### Step 4: 2,6-Dichloro-4-[difluoro-(1-methyl-3,6-dihydro-2H-pyridin-4-yl)methyl]pyridine

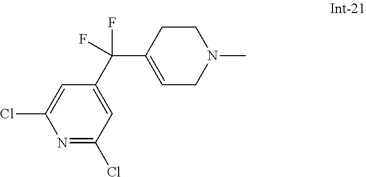 [SMILES: CN1CC=C(C(F)(F)c2cc(Cl)nc(Cl)c2)CC1]

To a solution of Int-21c (100.0 mg, 0.240 mmol) in methanol (2 mL) was added NaBH₄ (9.07 mg, 0.240 mmol) and kept stirring at 25° C. for 18 h. After the reaction was completed, the reaction mixture was quenched with water (50 mL) and extracted with EtOAc (30 mL×3). The combined organic layer was concentrated in vacuo to give the crude product, which was purified by silica-gel chromatography (elute with MeOH:DCM=0-5%) to give Int-21 (80 mg, 96.7% yield) as a light brown solid. MS (ESI⁺) obsd. [(M+H)⁺]: 293.1.

### Intermediate 22: tert-Butyl 4-[(2,6-dichloro-4-pyridyl)-difluoro-methyl]piperidine-1-carboxylate

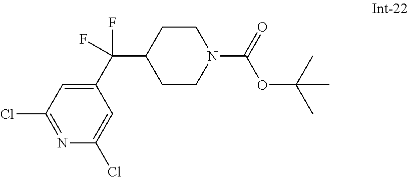 [SMILES: CC(C)(C)OC(=O)N1CCC(C(F)(F)c2cc(Cl)nc(Cl)c2)CC1]

The title compound was prepared in analogy to the preparation of Int-18 by using tert-butyl 4-formylpiperidine-1-carboxylate instead of 1,4-dioxane-2-carbaldehyde in Step 2. Compound Int-22 (5.62 g) was obtained as a white solid. MS (ESI⁺) obsd. [(M+H)⁺]: 381.2.

### Intermediate 23: tert-Butyl(2R)-2-[(2,6-dichloro-4-pyridyl)-difluoro-methyl]morpholine-4-carboxylate

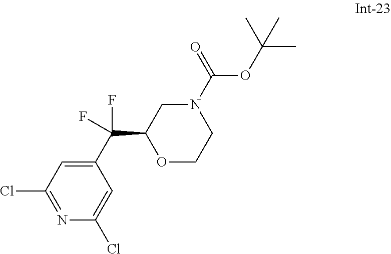 [SMILES: CC(C)(C)OC(=O)N1CCO[C@@H](C(F)(F)c2cc(Cl)nc(Cl)c2)C1]

The title compound was prepared in analogy to the preparation of Int-18 by using (R)—N-Boc-2-hydroxymethylmorpholine instead of 1,4-dioxan-2-ylmethanol in Step 1. Compound Int-23 (2.2 g, 96% ee) was obtained as a white solid. MS (ESI⁺) obsd. [(M+H)⁺]: 383.2.

### Intermediate Int-24: tert-Butyl(2S)-2-[(2,6-dichloro-4-pyridyl)-difluoro-methyl]morpholine-4-carboxylate

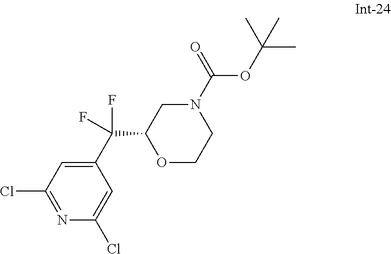 [SMILES: CC(C)(C)OC(=O)N1CCO[C@H](C(F)(F)c2cc(Cl)nc(Cl)c2)C1]

The title compound was prepared in analogy to the preparation of Int-18 by using(S)—N-Boc-2-hydroxymethylmorpholine instead of 1,4-dioxan-2-ylmethanol in Step 1. Compound Int-24 (1.2 g, 95% ee) was obtained as a white solid. MS (ESI⁺) obsd. [(M+H)⁺]: 383.2.

### Intermediate 25: tert-Butyl 3-[(2,6-dichloro-4-pyridyl)-difluoro-methyl]piperidine-1-carboxylate

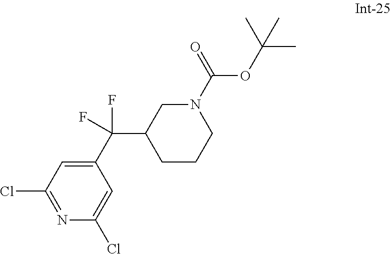 [SMILES: CC(C)(C)OC(=O)N1CCCC(C(F)(F)c2cc(Cl)nc(Cl)c2)C1]

The title compound was prepared in analogy to the preparation of Int-18 by using tert-butyl 3-(hydroxymethyl) piperidine-1-carboxylate instead of 1,4-dioxan-2-ylmethanol in Step 1. Compound Int-25 (3.1 g) was obtained as a yellow oil. MS (ESI⁺) obsd. [(M+H)⁺]: 359.0.

### Intermediate 26: 2,6-Dichloro-4-[difluoro(tetrahydropyran-4-yl)methyl]pyridine

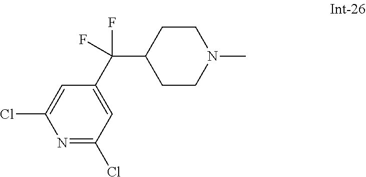 [SMILES: CN1CCC(C(F)(F)c2cc(Cl)nc(Cl)c2)CC1]

### Step 1: N-methoxy-N,1-dimethyl-piperidine-4-carboxamide

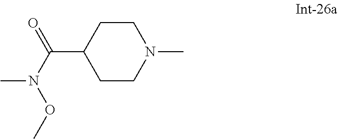

A mixture of 1-methylpiperidine-4-carboxylic acid (5.0 g, 34.92 mmol), O,N-dimethylhydroxylamine·HCl (5.11 g, 52.38 mmol), HATU (33.2 g, 87.3 mmol) and TEA (14.56 mL, 104.76 mmol) in DCM (100 mL) was stirred at 25° C. for 16 h. After the reaction was completed, the mixture was concentrated in vacuo to give a residue, which was purified by silica-gel chromatography (elute with MeOH:DCM=0-25%) to give Int-26a (1.6 g, 15% yield) as a light brown oil. MS obsd. (ESI⁺) [(M+H)⁺]: 187.2.

### Step 2: (2,6-Dichloro-4-pyridyl)-(1-methyl-4-piperidyl)methanone

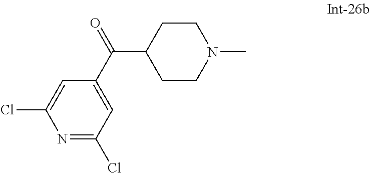 [SMILES: CN1CCC(C(=O)c2cc(Cl)nc(Cl)c2)CC1]

To a solution of n-butyllithium (6.26 mL, 15.03 mmol) in THF (10 mL) was added a solution of 2,6-dichloropyridine (0.68 mL, 7.52 mmol) in THF (10 mL) at −78° C. under N₂. After stirring for 30 minutes, a solution of Int-26a (1.4 g, 7.52 mmol) in THF (10 mL) was added and the mixture was stirred at −78° C. for 6 h. After the reaction was completed, the reaction mixture was quenched with aq. NH₄Cl (100 mL) and extracted with EtOAc (50 mL×3). The combined organic layer was washed with brine (80 mL), dried over anhydrous sodium sulfate, filtered and concentrated in vacuo to give a residue, which was purified by silica-gel chromatography (elute with EtOAc:PE=0-40%) to give Int-26b (200 mg, 7.31% yield) as light brown oil. MS obsd. (ESI⁺) [(M+H)⁺]: 273.2.

### Step 3: 2,6-Dichloro-4-[difluoro-(1-methyl-4-piperidyl)methyl]pyridine

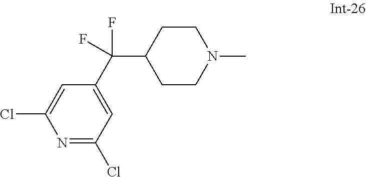 [SMILES: CN1CCC(C(F)(F)c2cc(Cl)nc(Cl)c2)CC1]

The title compound was prepared in analogy to the preparation of Int-18 by using Int-26b instead of Int-18c in Step 4. Compound Int-26 (200 mg) was obtained as a as light brown oil. MS (ESI⁺) obsd. [(M+H)⁺]: 295.1.

### Intermediate 27: tert-Butyl 6-[(2,6-dichloro-4-pyridyl)-difluoro-methyl]-3-azabicyclo[3.1.0]hexane-3-carboxylate

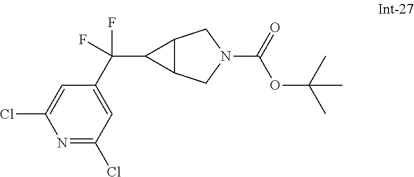 [SMILES: CC(C)(C)OC(=O)N1CC2C(C1)C2C(F)(F)c1cc(Cl)nc(Cl)c1]

The title compound was prepared in analogy to the preparation of Int-18 by using [(1S,5R)-3-azabicyclo[3.1.0]hexan-6-yl]methanol instead of 1,4-dioxan-2-ylmethanol in Step 1. Compound Int-27 (6.3 g) was obtained as a yellow solid. MS (ESI⁺) obsd. [(M+H)⁺]: 379.2.

### Intermediate 28: tert-Butyl 3-[(2,6-dichloro-4-pyridyl)-difluoro-methyl]azetidine-1-carboxylate

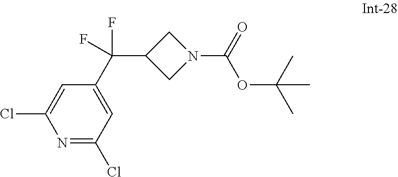 [SMILES: CC(C)(C)OC(=O)N1CC(C(F)(F)c2cc(Cl)nc(Cl)c2)C1]

The title compound was prepared in analogy to the preparation of Int-18 by using tert-butyl 3-(hydroxymethyl)azetidine-1-carboxylate instead of (1,4-dioxan-2-yl)methanol in Step 1. Int-28 (1.1 g) was obtained as a white solid. MS (ESI⁺) obsd. [(M+H)⁺]: 353.2.

### Intermediate 29: tert-Butyl 1-[(2,6-dichloro-4-pyridyl)-difluoro-methyl]-3-azabicyclo[3.1.0]hexane-3-carboxylate

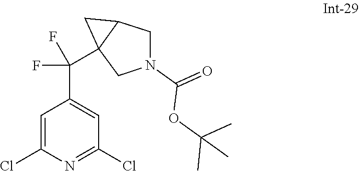 [SMILES: CC(C)(C)OC(=O)N1CC2CC2(C(F)(F)c2cc(Cl)nc(Cl)c2)C1]

The title compound was prepared in analogy to the preparation of Int-18 by using tert-butyl 1-(hydroxymethyl)-3-azabicyclo[3.1.0]hexane-3-carboxylate instead of (1,4-dioxan-2-yl)methanol in Step 1. Int-29 (2.6 g) was obtained as a white solid. MS (ESI⁺) obsd. [(M+H)⁺]: 379.2.

### Intermediate 30: tert-Butyl 6-[(2,6-dichloro-4-pyridyl)-difluoro-methyl]-2-azaspiro[3.3]heptane-2-carboxylate

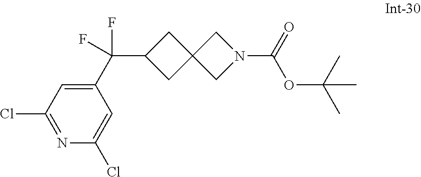 [SMILES: CC(C)(C)OC(=O)N1CC2(CC(C(F)(F)c3cc(Cl)nc(Cl)c3)C2)C1]

The title compound was prepared in analogy to the preparation of Int-18 by using tert-butyl 6-(hydroxymethyl)-2-azaspiro[3.3]heptane-2-carboxylate instead of (1,4-dioxan-2-yl)methanol in Step 1. Int-30 (195 mg) was obtained as a yellow solid. MS (ESI⁺) obsd. [(M+H)⁺]: 393.2.

### Intermediate 31: tert-Butyl 2-[(2,6-dichloro-4-pyridyl)-difluoro-methyl]-5-methyl-morpholine-4-carboxylate

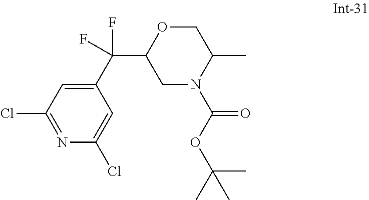 [SMILES: CC1COC(C(F)(F)c2cc(Cl)nc(Cl)c2)CN1C(=O)OC(C)(C)C]

The title compound was prepared in analogy to the preparation of Int-18 by using tert-butyl 2-(hydroxymethyl)-5-methyl-morpholine-4-carboxylate instead of (1,4-dioxan-2-yl)methanol in Step 1. Int-31 (580 mg) was obtained as a yellow solid. MS (ESI⁺) obsd. [(M+H)⁺]: 397.2.

### Intermediate 32: tert-Butyl N-[2-[3-[(2,6-dichloro-4-pyridyl)-difluoro-methyl]azetidin-1-yl]-1-methyl-2-oxo-ethyl]carbamate

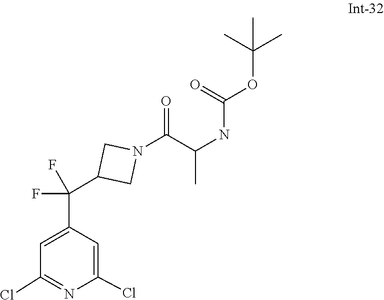 [SMILES: CC(NC(=O)OC(C)(C)C)C(=O)N1CC(C(F)(F)c2cc(Cl)nc(Cl)c2)C1]

### Step 1. 4-[Azetidin-3-yl(difluoro)methyl]-2,6-dichloro-pyridine

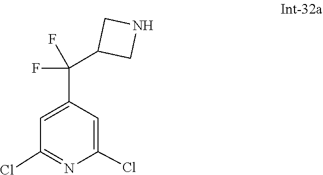 [SMILES: FC(F)(c1cc(Cl)nc(Cl)c1)C1CNC1]

A mixture of Int-28 (77.46 mg, 0.219 mmol) in TFA (500.12 mg, 4.39 mmol) and dichloromethane (1 mL) was stirred at room temperature for one hour. After the reaction was completed, the mixture was concentrated in vacuo to give crude Int-32a (55 mg, 99%). MS (ESI⁺) obsd. [(M+H)⁺]: 253.0.

### Step 2. tert-Butyl N-[2-[3-[(2,6-dichloro-4-pyridyl)-difluoro-methyl]azetidin-1-yl]-1-methyl-2-oxo-ethyl]carbamate

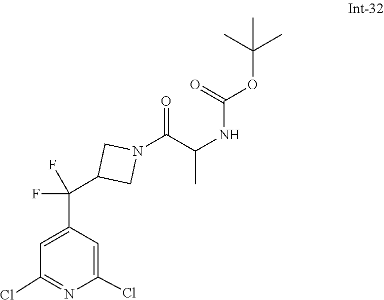 [SMILES: CC(NC(=O)OC(C)(C)C)C(=O)N1CC(C(F)(F)c2cc(Cl)nc(Cl)c2)C1]

A mixture of Int-32a (65 mg, 0.257 mmol), 2-(tert-butoxycarbonylamino) propionic acid (72.89 mg, 0.385 mmol), HATU (146.49 mg, 0.385 mmol) and DIPEA (165.97 mg, 1.28 mmol) in N,N-dimethylformamide (1.5 mL) was stirred at room temperature overnight. After the reaction was completed, the mixture was concentrated in vacuo to give crude product, which was purified by silica-gel chromatography (eluted with EtOAc:PE=0-35%) to afford Int-32 (100 mg, 92% yield). MS (ESI⁺) obsd. [(M+H)⁺]: 424.2.

### Intermediate 33: tert-Butyl N-[3-[[(E)-4-(2,6-dichloro-4-pyridyl)-4,4-difluoro-but-2-enoyl]amino]propyl]carbamate

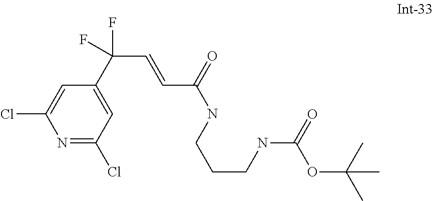

### Step 1. Ethyl 2-(2,6-dichloro-4-pyridyl)-2,2-difluoro-acetate

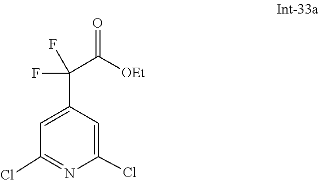 [SMILES: CCOC(=O)C(F)(F)c1cc(Cl)nc(Cl)c1]

A degassed mixture of 4-bromo-2,6-dichloro-pyridine (8.0 g, 35.26 mmol), ethyl bromodifluoroacetate (8.59 g, 42.31 mmol) and Cu (5.38 g, 84.62 mmol) in DMSO (30 mL) was stirred at 50° C. for 2 h under nitrogen. After the reaction was completed, the mixture was filtered and the filtrated was concentrated in vacuo to give a residue, which was purified by silica-gel chromatography (eluted with EtOAc:PE=0-20%) to give Int-33a (7.5 g, 78.76% yield) as colorless oil. MS (ESI⁺) obsd. [(M+H)⁺]: 269.8.

### Step 2. 2-(2,6-Dichloro-4-pyridyl)-2,2-difluoro-ethanol

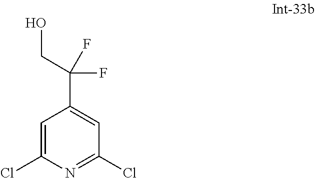 [SMILES: OCC(F)(F)c1cc(Cl)nc(Cl)c1]

To a solution of Int-33a (6.0 g, 22.22 mmol) in ethanol (20 mL) was added sodium borohydride (1.01 g, 26.66 mmol) slowly at 0° C. Then the mixture was slowly warmed up to 25° C. and stirred for 2 h. After the reaction was completed, the mixture was poured into ice-water (100 mL) and extracted with EtOAc (150 mL×3). The organic layer concentrated in vacuo to give crude Int-33b (4.8 g, 94.75% yield) as colorless oil without further purification. MS (ESI⁺) obsd. [(M+H)⁺]: 227.7.

### Step 3. 2-(2,6-Dichloro-4-pyridyl)-2,2-difluoro-acetaldehyde

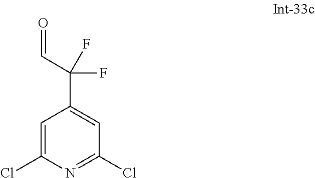 [SMILES: O=CC(F)(F)c1cc(Cl)nc(Cl)c1]

To a solution of Int-33b (2.5 g, 10.96 mmol) in DCM (20 mL) were added Dess-Martin periodinane (5.12 g, 12.06 mmol) at 0° C. Then the mixture was slowly warmed up to 25° C. and stirred for 2 h. After the reaction was completed, the mixture was poured into ice-water (100 mL) and extracted with EtOAc (150 mL×3). The organic layer was concentrated in vacuo to give crude product, which was purified by silica-gel chromatography (elute with EtOAc:PE=0-30%) to give crude Int-33c (1.8 g, 72.6% yield) as colorless oil without further purification. MS (ESI⁺) obsd. [(M+H)⁺]: 227.0.

### Step 4. Ethyl (E)-4-(2,6-dichloro-4-pyridyl)-4,4-difluoro-but-2-enoate

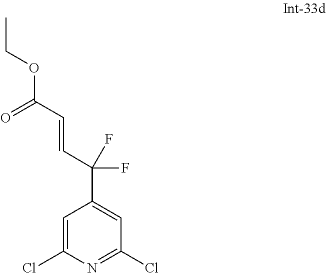 [SMILES: CCOC(=O)/C=C/C(F)(F)c1cc(Cl)nc(Cl)c1]

A mixture of Int-33c (1.8 g, 7.96 mmol) and (carbethoxymethylene)triphenylphosphorane (5.55 g, 15.93 mmol) in THF (15 mL) was stirred at 70° C. for 5 h under nitrogen. After the reaction was completed, the mixture was poured into water (100 mL and extracted with EtOAc (150 mL×3). The organic layer was concentrated in vacuo to give crude product, which was purified by silica-gel chromatography (elute with EtOAc:PE=0-20%) to give Int-33d (1.5 g, 60.3% yield) as colorless oil. MS (ESI⁺) obsd. [(M+H)⁺]: 295.8.

### Step 5. (E)-4-(2,6-dichloro-4-pyridyl)-4,4-difluoro-but-2-enoic acid (Int-33e)

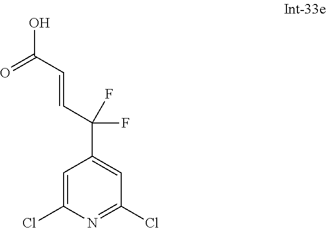 [SMILES: O=C(O)/C=C/C(F)(F)c1cc(Cl)nc(Cl)c1]

A mixture of Int-33d (200 mg, 675 μmol) and LiOH monohydrate (284 mg, 6.75 mmol) in THF (5 mL) and Water (5 mL) was stirred at rt for 4 h. After the reaction was completed, the mixture was acidified by 1N HCl and extracted with EtOAc (50 mL×3). The combined organic layer was concentrated under vacuum to give crude Int-33e (150 mg, 82.8% yield), which was used in the next step directly. MS (ESI⁺) obsd. [(M+H)⁺]: 268.0.

### Step 6. tert-Butyl N-[3-[[(E)-4-(2,6-dichloro-4-pyridyl)-4,4-difluoro-but-2-enoyl]amino]propyl]carbamate

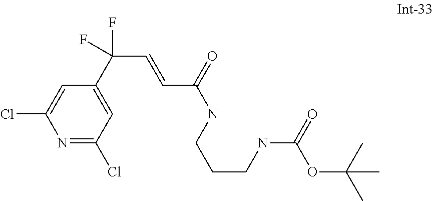

A mixture of Int-33e (60 mg, 0.224 mmol), N-(3-aminopropyl) carbamic acid tert-butyl ester (58.5 mg, 0.336 mmol) and DIPEA (86.79 mg, 0.672 mmol) and HATU (153.2 mg, 0.403 mmol) in N,N-dimethylformamide (1.5 mL) was stirred at room temperature for 16 h. After the reaction was completed, the mixture was concentrated in vacuo to give crude product, which was purified by silica-gel chromatography (eluted with EtOAc:PE=0-20%) to give Int-33 (85 mg, 89.0%). MS (ESI⁺) obsd. [(M+H)⁺]: 424.2.

### Intermediate 34: tert-Butyl N-[3-[[4-[(2,6-dichloro-4-pyridyl)-difluoro-methyl]cyclohexanecarbonyl]amino]propyl]carbamate

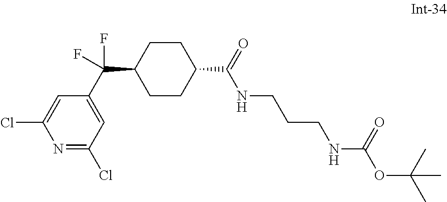 [SMILES: CC(C)(C)OC(=O)NCCCNC(=O)[C@H]1CC[C@H](C(F)(F)c2cc(Cl)nc(Cl)c2)CC1]

### Step 1: trans-Methyl 4-(hydroxymethyl)cyclohexanecarboxylate

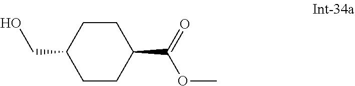

A mixture of trans-4-methoxycarbonylcyclohexanecarboxylic acid (50.0 g, 268.51 mmol) and BH₃·Me₂S (53.7 mL, 537.03 mmol) in THF (150 mL) was stirred at 25° C. for 12 h. After the reaction was completed, the mixture was quenched with methanol (100 mL) and concentrated in vacuo to give a crude residue, which was purified by silica-gel chromatography (elute with EtOAc:PE=0-50%) to give Int-34a (28 g, 60.6% yield) as colorless oil.

### Step 2: trans-Methyl 4-formylcyclohexanecarboxylate

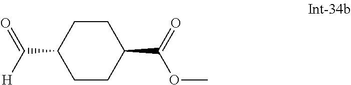

The title compound was prepared in analogy to the preparation of Int-18a by using Int-34a instead of 1,4-dioxan-2-ylmethanol. Int-34b (24 g) was obtained as a yellow oil.

### Step 3: Methyl 4-[(2,6-dichloro-4-pyridyl)-hydroxy-methyl]cyclohexanecarboxylate

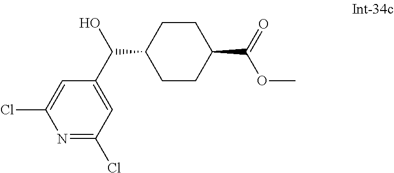 [SMILES: COC(=O)[C@H]1CC[C@H](C(O)c2cc(Cl)nc(Cl)c2)CC1]

The title compound was prepared in analogy to the preparation of Int-18b by using Int-34b instead of Int-18a. Int-34c (33 g) was obtained as a white solid. MS (ESI⁺) obsd. [(M+H)⁺]: 317.8.

### Step 4: trans-Methyl 4-(2,6-dichloropyridine-4-carbonyl)cyclohexanecarboxylate

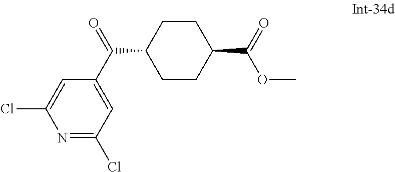 [SMILES: COC(=O)[C@H]1CC[C@H](C(=O)c2cc(Cl)nc(Cl)c2)CC1]

The title compound was prepared in analogy to the preparation of Int-18c by using Int-34c instead of Int-18b. Int-34d (30 g) was obtained as a white solid. MS (ESI⁺) obsd. [(M+H)⁺]: 315.8.

### Step 5: trans-Methyl 4-[(2,6-dichloro-4-pyridyl)-difluoro-methyl]cyclohexanecarboxylate

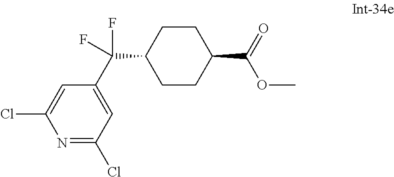 [SMILES: COC(=O)[C@H]1CC[C@H](C(F)(F)c2cc(Cl)nc(Cl)c2)CC1]

The title compound was prepared in analogy to the preparation of Int-18 by using Int-34d instead of Int-18c in step 4. Int-34e (25 g) was obtained as a white solid. MS (ESI⁺) obsd. [(M+H)⁺]: 337.8.

### Step 6: trans-4-[(2,6-Dichloro-4-pyridyl)-difluoro-methyl]cyclohexanecarboxylic acid

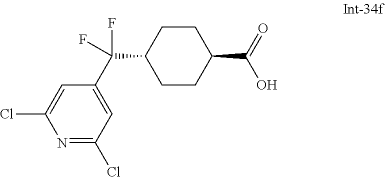 [SMILES: O=C(O)[C@H]1CC[C@H](C(F)(F)c2cc(Cl)nc(Cl)c2)CC1]

A mixture of Int-34e (2.0 g, 5.91 mmol), lithium bromide (5.1 g, 60 mmol) and TEA (3.0 mL, 21.52 mmol) in MeCN (10 mL) and water (3 mL) was stirred at 50° C. for 12 h. After the reaction was completed, the mixture was adjusted to pH=5 with 1 N HCl solution and extracted with EtOAc (300 mL×2). The combined organic layer was dried over anhydrous sodium sulfate, filtered and concentrated in vacuo to give crude Int-34f (1.8 g, 93.9% yield) as a white solid. MS (ESI⁺) obsd. [(M+H)⁺]: 323.8.

### Step 7: trans-N-butyl-4-[(2,6-dichloro-4-pyridyl)-difluoro-methyl]cyclohexanecarboxamide

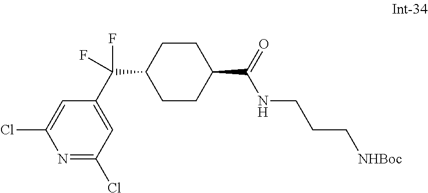 [SMILES: CC(C)(C)OC(=O)NCCCNC(=O)[C@H]1CC[C@H](C(F)(F)c2cc(Cl)nc(Cl)c2)CC1]

A mixture of Int-34f (150 mg, 463 μmol), HATU (264 mg, 694 μmol), tert-butyl N-(3-aminopropyl)carbamate (96.8 mg, 555 μmol) and DIPEA (242 μL, 1.39 mmol) in DMF (5 mL) was stirred at room temperature for 2 h. After the reaction was completed, the reaction mixture was diluted with EtOAc (10 mL) and washed with brine (50 mL). The organic layer was concentrated in vacuo to give crude product, which was purified by silica-gel chromatography (elute with EtOAc:PE=0-40%) to give Int-34 (180 mg, 81% yield) as yellow oil. MS (ESI⁺) obsd. [(M+H)⁺]: 480.6.

### Intermediate 35: tert-Butyl N-[3-[3-(tert-butoxycarbonylamino)propyl-[4-[(2,6-dichloro-4-pyridyl)-difluoro-methyl]cyclohexanecarbonyl]amino]propyl]carbamate

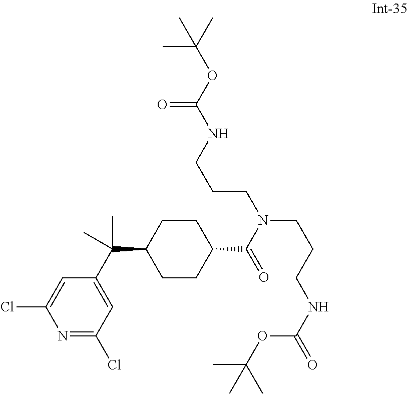 [SMILES: CC(C)(C)OC(=O)NCCCN(CCCNC(=O)OC(C)(C)C)C(=O)[C@H]1CC[C@H](C(C)(C)c2cc(Cl)nc(Cl)c2)CC1]

### Step 1: tert-Butyl N-[3-[3-(tert-butoxycarbonylamino)propyl-[4-[(2,6-dichloro-4-pyridyl)-difluoro-methyl]cyclohexanecarbonyl]amino]propyl]carbamate

A mixture of DIPEA (113.63 mg, 153.56 μL, 0.879 mmol), Int-34f (95 mg, 0.293 mmol) and HATU (167.15 mg, 0.440 mmol) in N,N-dimethylformamide (1.5 mL) was stirred at 25° C. for 16 h. After the reaction was completed, the mixture was diluted with EtOAc (20 mL), washed with sat. NH₄Cl aqueous (20 mL), dried over anhydrous Na₂SO₄ and filtered. The filtrate was concentrated in vacuo to give crude product (186 mg, 100%), which was used directly in next step. MS (ESI⁺) obsd. [(M+H)⁺]: 637.3.

### Intermediate 36:2-[bis[2-(tert-Butoxycarbonylamino)ethyl]amino]acetic acid

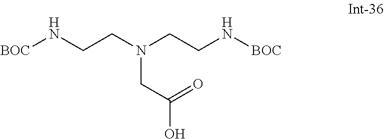 [SMILES: CC(C)(C)OC(=O)NCCN(CCNC(=O)OC(C)(C)C)CC(=O)O]

### Step 1. Methyl 2-[bis[2-(tert-butoxycarbonylamino)ethyl]amino]acetate

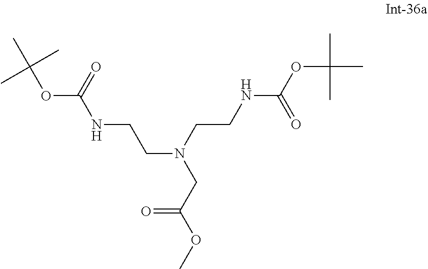 [SMILES: COC(=O)CN(CCNC(=O)OC(C)(C)C)CCNC(=O)OC(C)(C)C]

A mixture of methyl bromoacetate (100 mg, 0.654 mmol), DIPEA (168.97 mg, 1.31 mmol) and N-[2-[2-(tert-butoxycarbonylamino)ethylamino]ethyl]carbamic acid tert-butyl ester (198.34 mg, 0.654 mmol) in N, N-dimethylformamide (1.5 mL) was stirred at rt overnight. After the reaction was completed, the mixture was diluted with EtOAc (20 mL), washed with water (10 mL×2). The organic was concentrated in vacuo to give crude Int-36a (245 mg, 100%). MS (ESI⁺) obsd. [(M+H)⁺]: 376.0.

### Step 2. 2-[bis[2-(tert-Butoxycarbonylamino)ethyl]amino]acetic acid

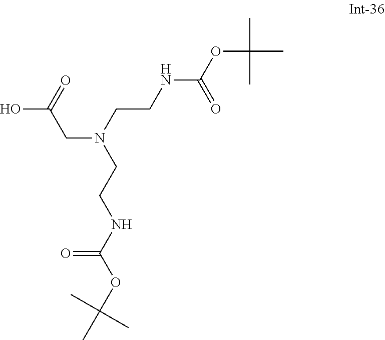 [SMILES: CC(C)(C)OC(=O)NCCN(CCNC(=O)OC(C)(C)C)CC(=O)O]

A mixture of Int-36a (245 mg, 0.653 mmol) and NaOH (261.01 mg, 6.53 mmol) in tetrahydrofuran (1.5 mL) and water (1.5 mL) was stirred at rt overnight. After the reaction was completed, the mixture was concentrated in vacuo to give crude Int-36 (230 mg, 97% yield). MS (ESI⁺) obsd. [(M+H)⁺]: 362.4.

### Intermediate 37: (2S)-1-(2-amino-2-oxo-ethyl)-N-[4-[(2,6-dichloro-4-pyridyl)-difluoro-methyl]cyclohexyl]-1-methyl-pyrrolidin-1-ium-2-carboxamide; chloride

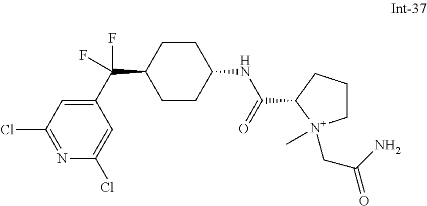 [SMILES: C[N+]1(CC(N)=O)CCC[C@H]1C(=O)N[C@H]1CC[C@H](C(F)(F)c2cc(Cl)nc(Cl)c2)CC1]

### Step 1: tert-Butyl N-(4-formylcyclohexyl)carbamate

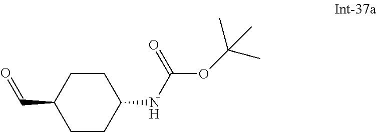 [SMILES: CC(C)(C)OC(=O)N[C@H]1CC[C@H](C=O)CC1]

Int-37a was prepared in analogy to the preparation of Int-18a by using compound tert-butyl N-[4-(hydroxymethyl)cyclohexyl]carbamate instead of 1,4-dioxan-2-ylmethanol. Int-37a (30 g) was obtained as a yellow oil.

### Step 2: tert-Butyl N-[4-[(2,6-dichloro-4-pyridyl)-hydroxy-methyl]cyclohexyl]carbamate

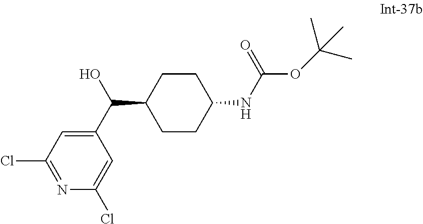 [SMILES: CC(C)(C)OC(=O)N[C@H]1CC[C@H](C(O)c2cc(Cl)nc(Cl)c2)CC1]

Int-37b was prepared in analogy to the preparation of Int-18b by using Int-37a instead of Int-18a. Int-37b (22 g) was obtained as a white solid. MS (ESI⁺) obsd. [(M+H)⁺]: 375.3.

### Step 3: tert-Butyl N-[4-(2,6-dichloropyridine-4-carbonyl)cyclohexyl]carbamate

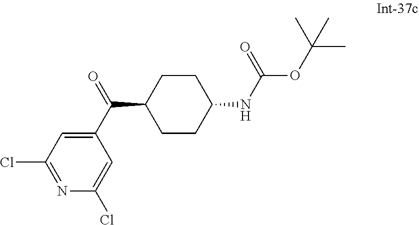 [SMILES: CC(C)(C)OC(=O)N[C@H]1CC[C@H](C(=O)c2cc(Cl)nc(Cl)c2)CC1]

Int-37c was prepared in analogy to the preparation of Int-18c by using Int-37b instead of Int-18b. Int-37c (17 g) was obtained as a light yellow solid. MS (ESI⁺) obsd. [(M+H)⁺]: 373.2.

### Step 4: tert-Butyl N-[4-[(2,6-dichloro-4-pyridyl)-difluoro-methyl]cyclohexyl]carbamate

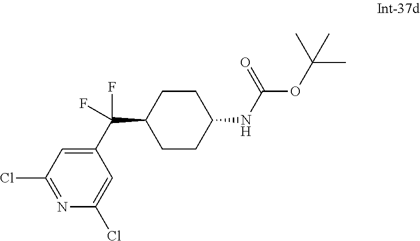 [SMILES: CC(C)(C)OC(=O)N[C@H]1CC[C@H](C(F)(F)c2cc(Cl)nc(Cl)c2)CC1]

Int-37d was prepared in analogy to the preparation of Int-18 by using Int-37c instead of Int-18c in step 4. Int-37d (11 g) was obtained as a light yellow solid. MS (ESI⁺) obsd. [(M+H)⁺]: 395.1.

### Step 5:4-[(2,6-Dichloro-4-pyridyl)-difluoro-methyl]cyclohexanamine

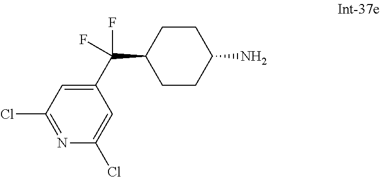 [SMILES: N[C@H]1CC[C@H](C(F)(F)c2cc(Cl)nc(Cl)c2)CC1]

A mixture of Int-37d (5.0 g, 12.65 mmol) in DCM (10 mL) and trifluoroacetic acid (10.0 mL, 129.8 mmol) was stirred at 25° C. for 1 h. After the reaction was completed, the mixture was concentrated to give crude Int-37e (3.5 g, 93.74% yield) as dark green oil. MS (ESI⁺) obsd. [(M+H)⁺]: 294.8.

### Step 6: (2S)—N-[4-[(2,6-dichloro-4-pyridyl)-difluoro-methyl]cyclohexyl]-1-methyl-pyrrolidine-2-carboxamide

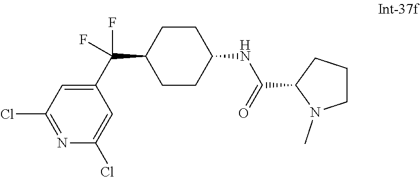 [SMILES: CN1CCC[C@H]1C(=O)N[C@H]1CC[C@H](C(F)(F)c2cc(Cl)nc(Cl)c2)CC1]

A mixture of(S)-1-methylpyrrolidine-2-carboxylic acid (157.53 mg, 1.22 mmol), Int-37e (180.0 mg, 0.610 mmol), HATU (347.82 mg, 0.910 mmol) and N,N-diisopropylethylamine (0.53 mL, 3.05 mmol) in DMF (2 mL) was stirred at 25° C. for 0.5 h. After the reaction was completed, the mixture was diluted with H₂O (200 mL), extracted with EA (200 mL×2). The organic layer was concentrated in vacuo to give Int-37f (200 mg, 80.7% yield) as a yellow solid. MS (ESI⁺) obsd. [(M+H)⁺]: 406.0.

### Step 7: (2S)-1-(2-amino-2-oxo-ethyl)-N-[4-[(2,6-dichloro-4-pyridyl)-difluoro-methyl]cyclohexyl]-1-methyl-pyrrolidin-1-ium-2-carboxamide;chloride

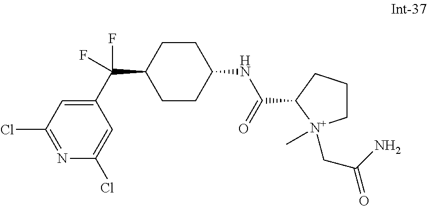 [SMILES: C[N+]1(CC(N)=O)CCC[C@H]1C(=O)N[C@H]1CC[C@H](C(F)(F)c2cc(Cl)nc(Cl)c2)CC1]

A mixture of Int-37f (300.0 mg, 0.740 mmol), 2-bromoacetamide (509.3 mg, 3.69 mmol), KI (612.85 mg, 3.69 mmol) and NaHCO₃ (310.12 mg, 3.69 mmol) in MeCN (5 mL) was stirred at 80° C. for 1 h. After the reaction was completed, the mixture was filtered and the filtrate was concentrated to give a crude residue which was purified by pre-HPLC (TFA as additive) to give Int-37 (120 mg, 35% yield) as a light yellow solid. MS (ESI⁺) obsd. [(M−Cl)⁺]: 463.0.

### Intermediate 38: Benzyl N-[2-[(2S)-2-[[4-[(2,6-dichloro-4-pyridyl)-difluoro-methyl]cyclohexyl]carbamoyl]pyrrolidin-1-yl]ethyl]carbamate

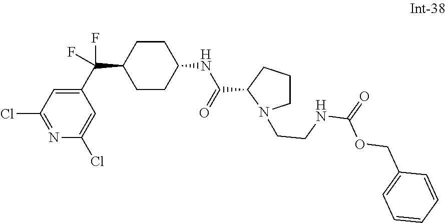 [SMILES: O=C(NCCN1CCC[C@H]1C(=O)N[C@H]1CC[C@H](C(F)(F)c2cc(Cl)nc(Cl)c2)CC1)OCc1ccccc1]

### Step 1: tert-Butyl(2S)-1-[2-(benzyloxycarbonylamino)ethyl]pyrrolidine-2-carboxylate

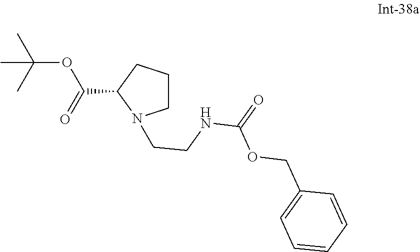 [SMILES: CC(C)(C)OC(=O)[C@@H]1CCCN1CCNC(=O)OCc1ccccc1]

To a solution of tert-butyl-(2S)-pyrrolidine-2-carboxylate (5.0 g, 29.2 mmol) and sodium hydrogen carbonate (7.36 g, 87.6 mmol) in MeCN (50 mL) and water (3 mL) was added benzyl N-(2-bromoethyl)carbamate (11.3 g, 43.8 mmol). The reaction was stirred at 25° C. for 24 h. After the reaction was completed, the reaction mixture was extracted with EtOAc (200 mL) and the organic layer was concentrated in vacuo to give a crude residue, which was purified by silica-gel chromatography (elute with EtOAc:PE=0-20%) to give Int-38a (8 g, 78.6% yield) as a light yellow oil. MS (ESI⁺) obsd. [(M+H)⁺]: 349.4.

### Step 2: (2S)-1-[2-(benzyloxycarbonylamino)ethyl]pyrrolidine-2-carboxylic acid

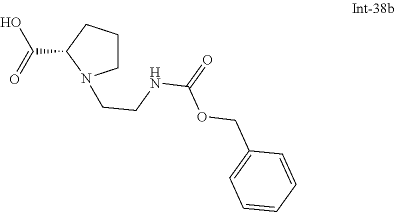 [SMILES: O=C(NCCN1CCC[C@H]1C(=O)O)OCc1ccccc1]

A solution of Int-38a (4.8 g, 13.78 mmol) in DCM (20 mL) and TFA (15.0 mL) was stirred at 20° C. for 30 h. After completion, solvent was evaporated to give crude Int-38b (4 g, 89.4% yield) as yellow oil. MS (ESI⁺) obsd. [(M+H)⁺]: 293.3.

### Step 3: Benzyl N-[2-[(2S)-2-[[4-[(2,6-dichloro-4-pyridyl)-difluoro-methyl]cyclohexyl]carbamoyl]pyrrolidin-1-yl]ethyl]carbamate

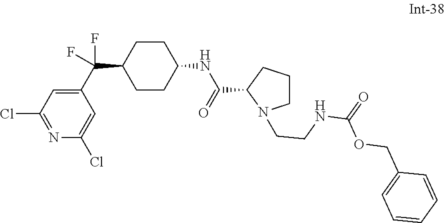 [SMILES: O=C(NCCN1CCC[C@H]1C(=O)N[C@H]1CC[C@H](C(F)(F)c2cc(Cl)nc(Cl)c2)CC1)OCc1ccccc1]

A mixture of Int-37e (300.0 mg, 1.03 mmol), HATU (585.37 mg, 1.54 mmol), Int-38b (121.16 mg, 0.410 mmol) and DIEA (0.2 mL) in DMF (10 mL) was stirred at 10° C. for 0.5 h. After the reaction was completed, the mixture was concentrated in vacuo to give crude product, which was purified by prep-HPLC (TFA as additive) to afford Int-38 (200 mg, 34.2% yield) as yellow solid. MS (ESI⁺) obsd. [(M+H)⁺]: 569.0.

### Intermediate 39:4-[(2,6-Dichloro-4-pyridyl)-difluoro-methyl]-N-(2-pyrrolidin-1-ylethyl)cyclohexanecarboxamide

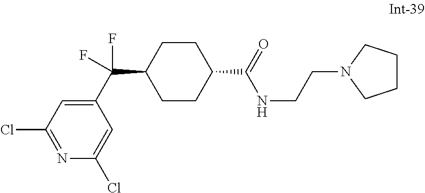 [SMILES: O=C(NCCN1CCCC1)[C@H]1CC[C@H](C(F)(F)c2cc(Cl)nc(Cl)c2)CC1]

### Step 1. N-[4-[(2,6-dichloro-4-pyridyl)-difluoro-methyl]cyclohexyl]-3-pyrrolidin-1-yl-propanamide

A mixture of 2-pyrrolidinoethylamine (33.47 mg, 0.293 mmol), DIPEA (113.63 mg, 153.56 μL, 0.879 mmol), 4-[(2,6-dichloro-4-pyridyl)-difluoro-methyl]cyclohexanecarboxylic acid (95 mg, 0.293 mmol) and HATU (167.15 mg, 0.440 mmol) in N,N-dimethylformamide (5 mL) was stirred at room temperature for 18 h. After the reaction was completed, the mixture was diluted with EtOAc (20 mL) and washed with water (20 mL). The organic was concentrated in vacuo to crude Int-39 (120 mg, 97%) give product without further purification. MS (ESI⁺) obsd. [(M+H)⁺]: 569.0.

### Example 001: trans-3-(aminomethyl)-7-[4-[6-chloro-4-(trifluoromethyl)-2-pyridyl]piperazin-1-yl]sulfonyl-3a,4-dihydro-3H-oxazolo[4,3-c][1,4]benzoxazin-1-one

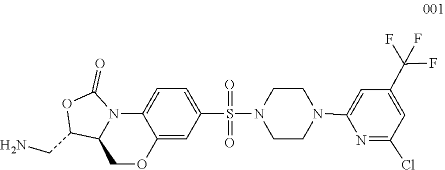 [SMILES: NC[C@@H]1OC(=O)N2c3ccc(S(=O)(=O)N4CCN(c5cc(C(F)(F)F)cc(Cl)n5)CC4)cc3OC[C@@H]12]

### Step 1: tert-butyl N-[[trans-7-[4-[6-chloro-4-(trifluoromethyl)-2-pyridyl]piperazin-1-yl]sulfonyl-1-oxo-3a,4-dihydro-3H-oxazolo[4,3-c][1,4]benzoxazin-3-yl]methyl]carbamate

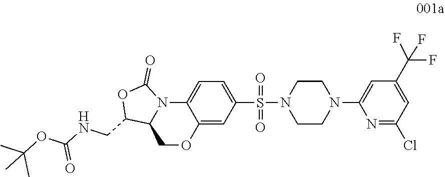 [SMILES: CC(C)(C)OC(=O)NC[C@@H]1OC(=O)N2c3ccc(S(=O)(=O)N4CCN(c5cc(C(F)(F)F)cc(Cl)n5)CC4)cc3OC[C@@H]12]

To a solution of compound int-7 (200.0 mg, 0.240 mmol) and Et₃N (393.44 mg, 3.89 mmol) in THF (1.11 mL) was added compound int-14 (63.43 mg, 0.240 mmol) at 25° C. and the mixture was stirred for 2 h. After the reaction was completed, solvent was removed under reduced pressure and the residue was purified by prep-HPLC (TFA as additive) to give compound 001a (20 mg, 6.5% yield) as a yellow solid. MS (ESI⁺) obsd. [(M+H)⁺]: 648.2.

### Step 2: trans-3-(aminomethyl)-7-[4-[6-chloro-4-(trifluoromethyl)-2-pyridyl]piperazin-1-yl]sulfonyl-3a,4-dihydro-3H-oxazolo[4,3-c][1,4]benzoxazin-1-one

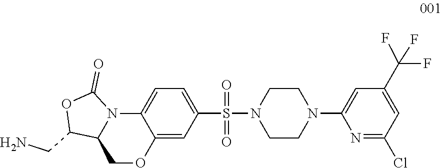 [SMILES: NC[C@@H]1OC(=O)N2c3ccc(S(=O)(=O)N4CCN(c5cc(C(F)(F)F)cc(Cl)n5)CC4)cc3OC[C@@H]12]

A mixture of compound 001a (30.0 mg, 0.046 mmol) in DCM (1 mL) and TFA (1.0 mL) was stirred at 20° C. for 1 h. After the reaction was completed, solvent was concentrated to give crude residue, which was purified by prep-HPLC (HCl as additive) to give Example 001 (5.1 mg, 17.2% yield) as a white solid. MS (ESI⁺) obsd. [(M+H)⁺]: 548.2.

Example 001: ¹H NMR (400 MHz, DMSO-d₆) δ 8.49-8.31 (m, 3H), 8.20-8.10 (m, 1H), 7.46-7.38 (m, 1H), 7.35-7.26 (m, 1H), 7.14-7.05 (m, 1H), 7.02-6.92 (m, 1H), 4.78-4.65 (m, 2H), 4.23-4.10 (m, 2H), 3.78-3.66 (m, 5H), 3.45-3.40 (m, 3H), 3.04-2.89 (m, 4H).

The following Examples 002 to 005 were prepared in analogy to the procedure described for the preparation of Example 001, replacing Int-14 with “Secondary amine”, and Int-9 with “RSO₂Cl” by the reagent indicated in Table 1 in Step 1.

TABLE 1
Compounds synthesis and characterization

| | | Secondary | |
|---|---|---|---|
| Example | Compounds Name and | amine and | |
| No. | Structure | ArSO₂Cl | NMR and (ESI⁺) |
| 002 | trans-3-(aminomethyl)-7-[4- | Secondary | ¹H NMR (400 MHz, |
| | [4-methyl-6- | amine: Int-15 | DMSO-d6) δ ppm 8.47- |
| | (trifluoromethyl)pyrimidin- | ArSO₂Cl: | 8.30 (m, 3H), 8.14 (d, |
| | 2-yl]piperazin-1-yl]sulfonyl- | Int-9 | J = 8.7 Hz, 1H), 7.40 |
| | 3a,4-dihydro-3H-oxazolo[4,3- | | (dd, J = 2.1, 8.6 Hz, |
| | c][1,4]benzoxazin-1-one | | 1H), 7.30 (d, J = 2.0 |
| | 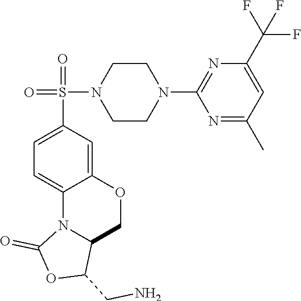 [SMILES: Cc1cc(C(F)(F)F)nc(N2CCN(S(=O)(=O)c3ccc4c(c3)OC[C@H]3[C@H](CN)OC(=O)N43)CC2)n1] | | Hz, 1H), 6.98 (s, 1H), 4.75-4.64 (m, 2H), 4.22-4.08 (m, 2H), 3.92-3.83 (m, 4H), 3.35-3.24 (m, 2H), 2.97 (br t, J = 4.5 Hz, 4H), 2.41-2.35 (m, 3H). MS (ESI⁺) obsd. [(M + H)⁺]: 529.1. |
| 003 | trans-3-(aminomethyl)-7-[4- | Secondary | ¹H NMR (400 MHz, |
| | (6-chloro-4- | amine: Int-16 | DMSO-d6) δ ppm 8.49- |
| | cyclopropylsulfonyl-2- | ArSO₂Cl: | 8.33 (m, 3H), 8.21- |
| | pyridyl)piperazin-1- | Int-9 | 8.10 (m, 1H), 7.46- |
| | yl]sulfonyl-3a,4-dihydro-3H- | | 7.35 (m, 1H), 7.33- |
| | oxazolo[4,3- | | 7.25 (m, 1H), 7.15- |
| | c][1,4]benzoxazin-1-one | | 7.08 (m, 1H), 7.05- |
| | 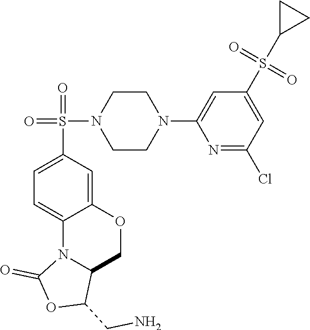 [SMILES: NC[C@@H]1OC(=O)N2c3ccc(S(=O)(=O)N4CCN(c5cc(S(=O)(=O)C6CC6)cc(Cl)n5)CC4)cc3OC[C@@H]12] | | 7.00 (m, 1H), 4.77- 4.63 (m, 2H), 4.25- 4.08 (m, 2H), 3.78- 3.63 (m, 4H), 3.48- 3.36 (m, 2H), 3.06- 2.93 (m, 5H), 1.18- 1.12 (m, 2H), 1.12- 1.04 (m, 2H). MS (ESI⁺) obsd. [(M + H)⁺]: 584.2. |
| 004 | trans-3-(aminomethyl)-7-[4- | Secondary | ¹H NMR (400 MHz, |
| | [4-(4-aminophenyl)sulfonyl- | amine: Int-17 | DMSO-d6)) δ ppm |
| | 6-chloro-2-pyridyl]piperazin- | ArSO₂Cl: | 8.56-8.40 (m, 6H), |
| | 1-yl]sulfonyl-3a,4-dihydro- | Int-9 | 8.22-8.10 (m, 2H), |
| | 3H-oxazolo[4,3- | | 7.58 (d, J = 8.8 Hz, |
| | c][1,4]benzoxazin-1-one | | 2H), 7.66-7.52 (m, |
| | 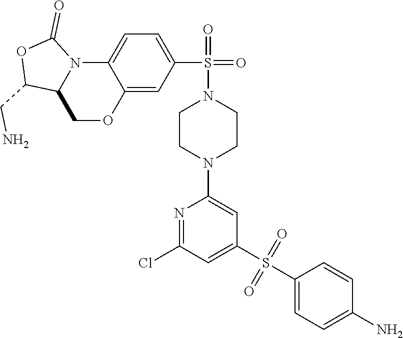 [SMILES: NC[C@@H]1OC(=O)N2c3ccc(S(=O)(=O)N4CCN(c5cc(S(=O)(=O)c6ccc(N)cc6)cc(Cl)n5)CC4)cc3OC[C@@H]12] | | 1H), 7.75-7.51 (m, 1H), 7.44-7.37 (m, 2H), 7.30 (s, 2H), 7.11- 7.02 (m, 2H), 6.91- 6.81 (m, 2H), 6.75- 6.48 (m, 4H), 4.79- 4.67 (m, 4H), 4.23- 4.10 (m, 5H), 2.97 (br t, J = 4.3 Hz, 8H), 2.08-2.06 (m, 1H). MS (ESI⁺) obsd. [(M + H)⁺]: 635.1. |
| 005 | cis-3-(aminomethyl)-7-[4-(6- | Secondary | ¹H NMR (400 MHz, |
| | chloro-4-cyclopropylsulfonyl- | amine: Int-16 | DMSO-d6) δ ppm 8.62- |
| | 2-pyridyl)piperazin-1- | ArSO₂Cl: | 8.27 (m, 3H), 8.26- |
| | yl]sulfonyl-3a,4-dihydro-3H- | Int-9 | 8.08 (m, 1H), 7.45- |
| | oxazolo[4,3- | | 7.38 (m, 1H), 7.34- |
| | c][1,4]benzoxazin-1-one | | 7.27 (m, 1H), 7.14- |
| | 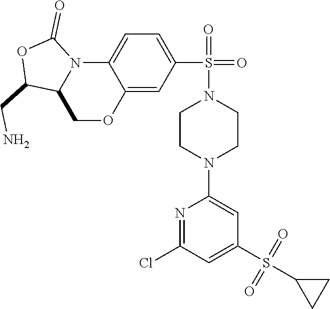 [SMILES: NC[C@H]1OC(=O)N2c3ccc(S(=O)(=O)N4CCN(c5cc(S(=O)(=O)C6CC6)cc(Cl)n5)CC4)cc3OC[C@@H]12] | | 7.09 (m, 1H), 7.03 (s, 1H), 5.31-4.97 (m, 1H), 4.75-4.60 (m, 1H), 4.58-4.44 (m, 1H), 4.21-4.07 (m, 1H), 3.71 (br d, J = 4.4 Hz, 4H), 3.28-3.23 (m, 1H), 3.18-3.11 (m, 1H), 2.99 (br s, 5H), 1.22-1.12 (m, 2H), 1.12-0.95 (m, 2H). MS (ESI⁺) obsd. [(M + H)⁺]: 585.2. |

### Example 006: cis-3-(aminomethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one

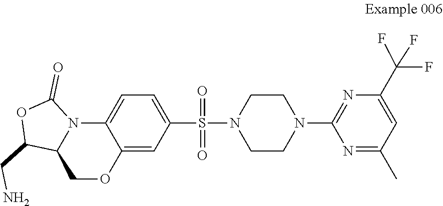 [SMILES: Cc1cc(C(F)(F)F)nc(N2CCN(S(=O)(=O)c3ccc4c(c3)OC[C@H]3[C@@H](CN)OC(=O)N43)CC2)n1]

### Step 1: tert-butyl N-[[(cis-7-[4-[4-methyl-6-(trifluoromethyl)pyrimidin-2-yl]piperazin-1-yl]sulfonyl-1-oxo-3a,4-dihydro-3H-oxazolo[4,3-c][1,4]benzoxazin-3-yl]methyl]carbamate

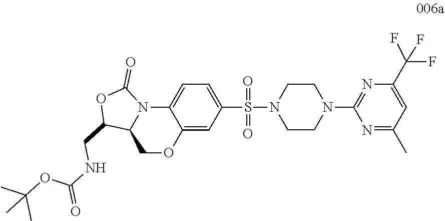 [SMILES: Cc1cc(C(F)(F)F)nc(N2CCN(S(=O)(=O)c3ccc4c(c3)OC[C@H]3[C@@H](CNC(=O)OC(C)(C)C)OC(=O)N43)CC2)n1]

A mixture of Int-6 (100.0 mg, 0.210 mmol), DIPEA (82.75 mg, 0.640 mmol) and 2-chloro-4-methyl-6-(trifluoromethyl)pyrimidine (62.93 mg, 0.320 mmol) in NMP (1 mL) was stirred at 75° C. for 12 h. After the reaction was completed, the mixture was poured into water (50 mL) and extracted with EtOAc (50 mL×3). The combined organic layer was concentrated in vacuo to give crude compound, which was purified by prep-HPLC (FA as additive) to afford compound 006a (110 mg, 81.9% yield) as a white solid. MS (ESI⁺) obsd. [(M+H)⁺]: 629.2.

### Step 2: cis-3-(aminomethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one

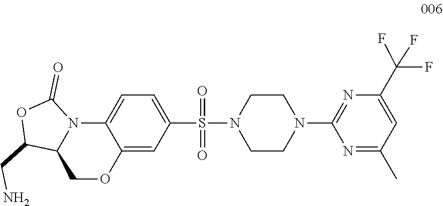 [SMILES: Cc1cc(C(F)(F)F)nc(N2CCN(S(=O)(=O)c3ccc4c(c3)OC[C@H]3[C@@H](CN)OC(=O)N43)CC2)n1]

To a solution of compound 006a (100.0 mg, 0.160 mmol) in DCM (2 mL) was added trifluoroacetic acid (1.0 mL, 12.98 mmol). The mixture was stirred at 25° C. for 2 h. After the reaction was completed, solvent was removed and the residue was purified by prep-HPLC (HCl as additive) to give Example 006 (61.9 mg, 68.57% yield) as a white solid. MS (ESI⁺) obsd. [(M+H)⁺]: 529.2.

Example 006: ¹H NMR (400 MHz, DMSO-d₆) δ ppm 8.47-8.63 (m, 3H), 8.14-8.22 (m, 1H), 7.37-7.43 (m, 1H), 7.29-7.33 (m, 1H), 6.96-7.00 (m, 1H), 5.15-5.24 (m, 1H), 4.66-4.74 (m, 1H), 4.43-4.55 (m, 1H), 4.08-4.19 (m, 1H), 3.80-3.92 (m, 4H), 3.12-3.31 (m, 2H), 2.93-3.02 (m, 4H), 2.36-2.41 (m, 3H). MS (ESI): 529.2 ([M+H]⁺).

The following Examples 007 to 010 were prepared in analogy to the procedure described for the preparation of Example 006, replacing 2-chloro-4-methyl-6-(trifluoromethyl)pyrimidine with “RCl”, and Int-8 with “Secondary amine” by the reagent indicated in Table 2 in Step 1.

TABLE 2
Compounds synthesis and characterization

| Example | Compounds Name and | Secondary | |
|---|---|---|---|
| No. | Structure | amine and RCl | NMR and (ESI⁺) |
| 007 | cis-3-(aminomethyl)-7-[4-[6- | Secondary | ¹H NMR (400 MHz, |
| | chloro-4-(trifluoromethyl)-2- | amine: Int-6 | DMSO-d6) δ ppm 8.47- |
| | pyridyl]piperazin-1- | RCl: | 8.71 (m, 3 H), 8.16- |
| | yl]sulfonyl-3a,4-dihydro-3H- | 2,6-dichloro-4- | 8.25 (m, 1 H), 7.38- |
| | oxazolo[4,3- | (trifluoromethyl) | 7.46 (m, 1 H), 7.29- |
| | c][1,4]benzoxazin-1-one | pyridine | 7.35 (m, 1 H), 7.08- |
| | 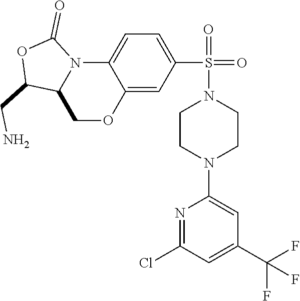 [SMILES: NC[C@H]1OC(=O)N2c3ccc(S(=O)(=O)N4CCN(c5cc(C(F)(F)F)cc(Cl)n5)CC4)cc3OC[C@@H]12] | | 7.14 (m, 1 H), 6.94- 7.00 (m, 1 H), 5.15- 5.26 (m, 1 H), 4.67- 4.76 (m, 1 H), 4.45- 4.58 (m, 1 H), 4.09- 4.21 (m, 1 H), 3.63- 3.78 (m, 4 H), 3.14- 3.31 (m, 2 H), 2.90- 3.04 (m, 4 H). MS (ESI⁺) obsd. [(M + H)⁺]: 548.1. |
| 008 | 3-Amino-7-[4-[6-chloro-4- | Secondary | ¹H NMR (400 MHz, |
| | [1,4-dioxan-2- | amine: Int-13 | CDCl₃) δ ppm 8.72 |
| | yl(difluoro)methyl]-2- | RCl: | (m, 1H), 7.36 (m, 2H), |
| | pyridyl]piperazin-1- | Int-18 | 6.68 (m, 1H), 6.50 (m, |
| | yl]sulfonyl-2,3,3a,4- | | 1H), 4.75 (m, 1H), |
| | tetrahydropyrrolo[2,1- | | 3.92 (m, 2H), 3.79 (t, |
| | c][1,4]benzoxazin-1-one | | J = 10.4 Hz, 2H), 3.74- |
| | 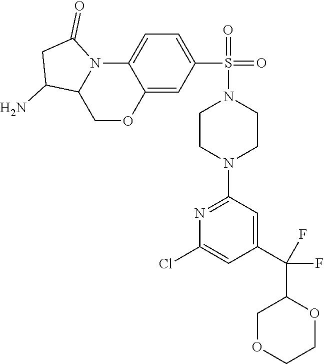 [SMILES: NC1CC(=O)N2c3ccc(S(=O)(=O)N4CCN(c5cc(C(F)(F)C6COCCO6)cc(Cl)n5)CC4)cc3OCC12] | | 3.52 (m, 9H), 3.45- 3.37 (m, 1H), 3.10 (t, J = 5.0 Hz, 4H), 2.86 (dd, J = 8.3, 16.7 Hz, 1H), 2.49 (m, 1H). MS (ESI⁺) obsd. [(M + H)⁺]: 600.0. |
| 009 | trans-3-(aminomethyl)-7-[4- | Secondary | ¹H NMR (400 MHz, |
| | [6-chloro-4-[difluoro(3- | amine: Int-8 | DMSO-d6) δ ppm |
| | piperidyl)methyl]-2- | RCl: Int-25 | 8.00-8.12 (m, 1H), |
| | pyridyl]piperazin-1- | | 7.29-7.38 (m, 1H), |
| | yl]sulfonyl-3a,4-dihydro-3H- | | 7.19-7.29 (m, 1H), |
| | oxazolo[4,3- | | 6.71-6.76 (m, 1H), |
| | c][1,4]benzoxazin-1-one | | 6.61-6.68 (m, 1H), |
| | 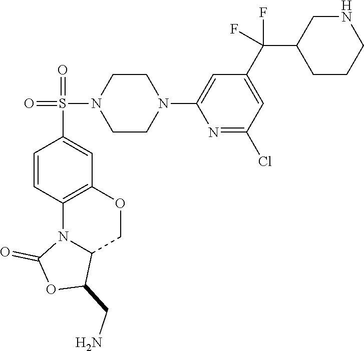 [SMILES: NC[C@H]1OC(=O)N2c3ccc(S(=O)(=O)N4CCN(c5cc(C(F)(F)C6CCCNC6)cc(Cl)n5)CC4)cc3OC[C@H]12] | | 4.54-4.63 (m, 1H), 4.42-4.54 (m, 1H), 3.98-4.12 (m, 2H), 3.53-3.67 (m, 4H), 3.03-3.14 (m, 6H), 2.86-2.98 (m, 5H), 2.55-2.70 (m, 3H), 2.23-2.29 (m, 1H), 1.60-1.76 (m, 2H), 1.37-1.51 (m, 1H), 1.20-1.35 (m, 1H). ¹⁹F NMR (376 MHz, DMSO-d6) δ ppm −110.41-−97.92 (m, 2 F). MS obsd. (ESI⁺) [(M + H)⁺]: 613.2 |
| 010 | cis-3-(aminomethyl)-7-[4-[6- | Secondary | ¹H NMR (400 MHz, |
| | chloro-4-[difluoro-(1-methyl- | amine: Int-6 | DMSO-d6) δ ppm |
| | 4-piperidyl)methyl]-2- | RCl: Int-26 | 8.15-8.22 (m, 1H), |
| | pyridyl]piperazin-1- | | 7.39-7.48 (m, 1H), |
| | yl]sulfonyl-3a,4-dihydro-3H- | | 7.30-7.36 (m, 1H), |
| | oxazolo[4,3- | | 6.80-6.88 (m, 1H), |
| | c][1,4]benzoxazin-1-one | | 6.71-6.77 (m, 1H), |
| | 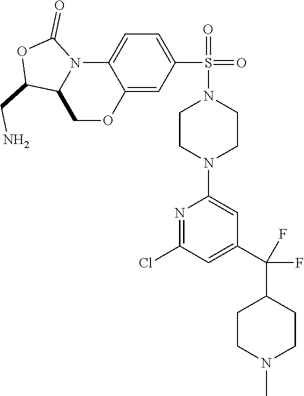 [SMILES: CN1CCC(C(F)(F)c2cc(Cl)nc(N3CCN(S(=O)(=O)c4ccc5c(c4)OC[C@H]4[C@@H](CN)OC(=O)N54)CC3)c2)CC1] | | 4.97-5.16 (m, 1H), 4.58-4.72 (m, 1H), 4.45-4.60 (m, 1H), 4.08-4.22 (m, 1H), 3.57-3.75 (m, 4H), 3.42-3.50 (m, 2H), 3.23-3.29 (m, 1H), 3.10-3.23 (m, 2H), 2.94-3.02 (m, 4H), 2.79-2.94 (m, 2H), 2.65-2.80 (m, 4H), 1.91-2.07 (m, 1H), 1.72-1.90 (m, 2H), 1.37-1.68 (m, 2H). 19F NMR (376 MHz, DMSO-d6) δ ppm −110.96-−100.14 (m, 2F) MS obsd. (ESI⁺) [(M + H)⁺]: 627.2. |

### Example 011: 7-[4-[4-Methyl-6-(trifluoromethyl)pyrimidin-2-yl]piperazin-1-yl]sulfonyl-2,3,3a,4-tetrahydropyrrolo[2,1-c][1,4]benzoxazin-1-one

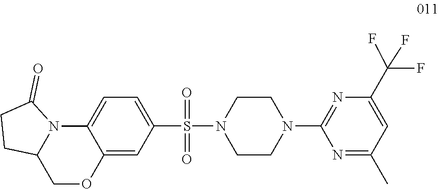 [SMILES: Cc1cc(C(F)(F)F)nc(N2CCN(S(=O)(=O)c3ccc4c(c3)OCC3CCC(=O)N43)CC2)n1]

A mixture of Int-12 (0.029 g, 86 μmol), 2-chloro-4-methyl-6-(trifluoromethyl)pyrimidine (16.9 mg, 86 μmol) and DIPEA (33.3 mg, 258 μmol) in NMP (2 mL) was stirred at 90° C. overnight. After the reaction was completed, the mixture was diluted with EtOAc (20 mL) and washed with water (20 mL). The organic phase was concentrated in vacuo to give crude residue, which was purified by HPLC (NH₃ as additive) to give Example 011 (2.5 mg, 6%). MS obsd. (ESI⁺) [(M+H)⁺]: 498.3.

Example 011: ¹H NMR (400 MHz, DMSO-d₆) δ ppm 8.61 (d, J=8.7 Hz, 1H), 7.33 (br d, J=8.7 Hz, 1H), 7.25 (s, 1H), 6.97 (s, 1H), 4.60 (br d, J=10.6 Hz, 1H), 4.04 (m, 1H), 3.84 (m, 5H), 2.97 (br s, 4H), 2.64 (td, J=10.1, 17.0 Hz, 1H), 2.38 (m, 4H), 2.22 (m, 1H), 1.69 (m, 1H).

The following Examples 012 to 016 were prepared in analogy to the procedure described for the preparation of Example 011, replacing 2-chloro-4-methyl-6-(trifluoromethyl)pyrimidine with “RCl”, and Int-12 with “Secondary amine” by the reagent indicated in Table 3 in Step 1.

TABLE 3
Compounds synthesis and characterization

| Example | Compounds Name and | Secondary | |
|---|---|---|---|
| No. | Structure | amine and RCl | NMR and (ESI⁺) |
| 012 | 7-[4-[6-Chloro-4- | Secondary | ¹H NMR (400 MHz, |
| | (trifluoromethyl)-2- | amine: Int-12 | DMSO-d6) δ ppm 8.55 |
| | pyridyl]piperazin-1- | RCl: | (dd, J = 1.5, 8.6 Hz, |
| | yl]sulfonyl-2,3,3a,4- | 2,6-dichloro-4- | 1H), 7.27 (br d, J = 8.6 |
| | tetrahydropyrrolo[2,1- | (trifluoromethyl) | Hz, 1H), 7.19 (s, 1H), |
| | c][1,4]benzoxazin-1-one | pyridine | 7.01 (s, 1H), 6.89 (s, |
| | 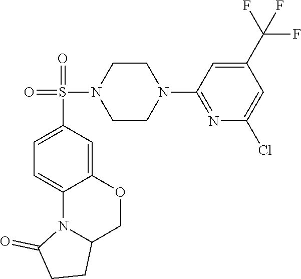 [SMILES: O=C1CCC2COc3cc(S(=O)(=O)N4CCN(c5cc(C(F)(F)F)cc(Cl)n5)CC4)ccc3N12] | | 1H), 4.53 (m, 1H), 3.98 (br d, J = 7.5 Hz, 1H), 3.77 (br t, J = 9.9 Hz, 1H), 3.64 (br s, 4H), 2.90 (br s, 4H), 2.57 (m, 1H), 2.30 (br dd, J = 9.7, 16.9 Hz, 1H), 2.16 (m, 1H), 1.62 (quin, J = 10.6 Hz, 1H). MS obsd. (ESI⁺) [(M + H)⁺]: 517.1. |
| 013 | 7-[4-[4-(4- | Secondary | ¹H NMR (400 MHz, |
| | Aminophenyl)sulfonyl-6- | amine: Int-12 | DMSO-d6) δ ppm 8.62 |
| | chloro-2-pyridyl]piperazin-1- | RCl: | (d, J = 8.7 Hz, 1H), |
| | yl]sulfonyl-2,3,3a,4- | 4-[(2,6- | 7.60-7.56 (m, 2H), |
| | tetrahydropyrrolo[2,1- | dichloro-4- | 7.33 (dd, J = 2.1, 8.6 |
| | c][1,4]benzoxazin-1-one | pyridyl)sulfonyl] | Hz, 1H), 7.24 (d, J = |
| | 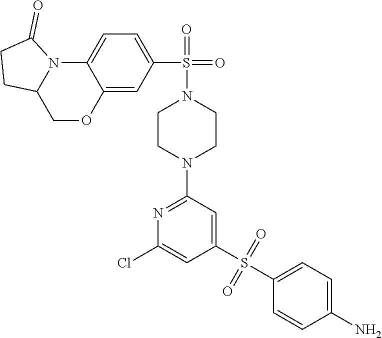 [SMILES: Nc1ccc(S(=O)(=O)c2cc(Cl)nc(N3CCN(S(=O)(=O)c4ccc5c(c4)OCC4CCC(=O)N54)CC3)c2)cc1] | aniline | 2.1 Hz, 1H), 7.08- 7.06 (m, 1H), 6.87 (d, J = 0.7 Hz, 1H), 6.64- 6.59 (m, 2H), 4.60 (dd, J = 3.1, 10.6 Hz, 1H), 4.10-4.00 (m, 1H), 3.88-3.80 (m, 1H), 3.69-3.64 (m, 4H), 2.96 (br s, 4H), 2.69- 2.58 (m, 1H), 2.42- 2.31 (m, 1H), 2.29- 2.15 (m, 1H), 1.76- 1.62 (m, 1H). MS obsd. (ESI⁺) [(M + H)⁺]: 604.2. |
| 014 | 7-[4-[4-(4- | Secondary | ¹H NMR (400 MHz, |
| | Aminophenyl)sulfonyl-6- | amine: Int-12 | DMSO-d6) δ ppm 8.62 |
| | bromo-2-pyridyl]piperazin-1- | RCl: | (d, J = 8.6 Hz, 1H), |
| | yl]sulfonyl-2,3,3a,4- | 4-[(2,6- | 7.61-7.55 (m, 2H), |
| | tetrahydropyrrolo[2,1- | dibromo-4- | 7.33 (dd, J = 2.1, 8.6 |
| | c][1,4]benzoxazin-1-one | pyridyl)sulfonyl] | Hz, 1H), 7.24 (d, J = |
| | 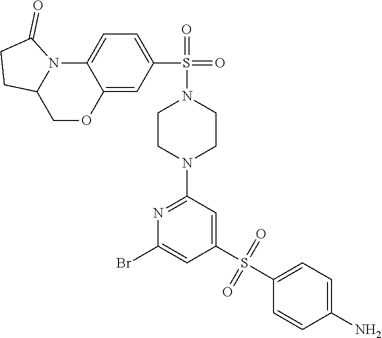 [SMILES: Nc1ccc(S(=O)(=O)c2cc(Br)nc(N3CCN(S(=O)(=O)c4ccc5c(c4)OCC4CCC(=O)N54)CC3)c2)cc1] | aniline | 2.1 Hz, 1H), 7.12- 7.06 (m, 1H), 6.98 (s, 1H), 6.68-6.55 (m, 2H), 4.60 (dd, J = 3.1, 10.8 Hz, 1H), 4.16- 3.94 (m, 1H), 3.84 (t, J = 10.3 Hz, 1H), 3.71- 3.59 (m, 4H), 2.96 (br t, J = 4.7 Hz, 4H), 2.62 (m, 1H), 2.37 (m, 1H), 2.33 (m, 1H), 1.78- 1.61 (m, 1H). MS obsd. (ESI⁺) [(M + H)⁺]: 648.4 |
| 015 | (3R,3aR)-3-(hydroxymethyl)- | Secondary | ¹H NMR (400 MHz, |
| | 7-[4-[4-methyl-6- | amine: Int-2B | DMSO-d6) δ ppm 8.22 |
| | (trifluoromethyl)pyrimidin- | RCl: | (d, J = 8.7 Hz, 1H), |
| | 2-yl]piperazin-1-yl]sulfonyl- | 2-chloro-4- | 7.37 (dd, J = 2.1, 8.6 |
| | 3a,4-dihydro-3H-oxazolo[4,3- | methyl-6- | Hz, 1H), 7.28 (d, J = |
| | c][1,4]benzoxazin-1-one | (trifluoromethyl) | 2.1 Hz, 1H), 6.97 (s, |
| | 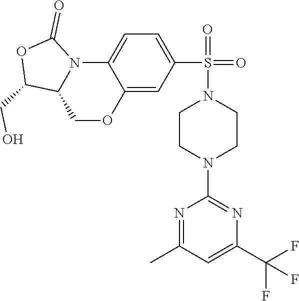 [SMILES: Cc1cc(C(F)(F)F)nc(N2CCN(S(=O)(=O)c3ccc4c(c3)OC[C@@H]3[C@H](CO)OC(=O)N43)CC2)n1] | pyrimidine | 1H), 4.84 (m, 1H), 4.68 (dd, J = 3.1, 10.5 Hz, 1H), 4.44 (m, 1H), 4.20(m, 1H), 3.87 (m, 4H), 3.70 (dd, J = 4.3, 12.4 Hz, 1H), 3.58 (dd, J = 3.8, 12.3 Hz, 1H), 2.98 (br t, J = 4.8 Hz, 4H), 2.38 (s, 3H) MS obsd. (ESI⁺) [(M + H)⁺]: 530.2 |
| 016 | 7-[4-[6-Chloro-4-[difluoro-(1- | Secondary | ¹H NMR (400 MHz, |
| | methyl-4-piperidyl)methyl]- | amine: Int-12 | DMSO-d6) δ ppm |
| | 2-pyridyl]piperazin-1- | RCl: | 8.47-8.68 (m, 1H), |
| | yl]sulfonyl-2,3,3a,4- | Int-26 | 7.30-7.43 (m, 1H), |
| | tetrahydropyrrolo[2,1- | | 7.20-7.31 (m, 1H), |
| | c][1,4]benzoxazin-1-one | | 6.77-6.84 (m, 1H), |
| | 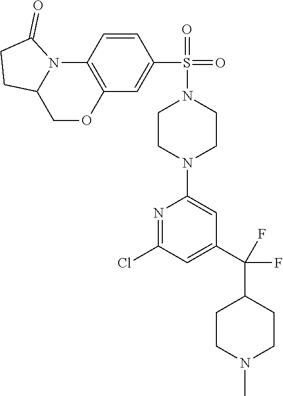 [SMILES: CN1CCC(C(F)(F)c2cc(Cl)nc(N3CCN(S(=O)(=O)c4ccc5c(c4)OCC4CCC(=O)N54)CC3)c2)CC1] | | 6.68-6.76 (m, 1H), 4.53-4.69 (m, 1H), 3.98-4.14 (m, 2H), 3.78-3.90 (m, 2H), 3.63-3.72 (m, 4H), 2.94-3.02 (m, 4H), 2.80-2.91 (m, 2H), 2.71-2.77 (m, 3H), 2.29-2.43 (m, 2H), 2.17-2.29 (m, 1H), 1.75-1.86 (m, 2H), 1.61-1.75 (m, 2H), 1.44-1.60 (m, 2H), 1.02-1.24 (m, 1H). ¹⁹F NMR (376 MHz, DMSO-d6) δ ppm −109.30-−100.97 (m, 2F). MS obsd. (ESI⁺) [(M + H)⁺]: 596.3 |

### Example 017: trans-3-(aminomethyl)-7-[4-[6-chloro-4-[1,4-dioxan-2-yl(difluoro)methyl]-2-pyridyl]piperazin-1-yl]sulfonyl-3a,4-dihydro-3H-oxazolo[4,3-c][1,4]benzoxazin-1-one

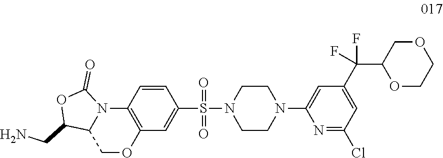 [SMILES: NC[C@H]1OC(=O)N2c3ccc(S(=O)(=O)N4CCN(c5cc(C(F)(F)C6COCCO6)cc(Cl)n5)CC4)cc3OC[C@H]12]

### Step 1: Benzyl N-[[trans-7-[4-[6-chloro-4-[1,4-dioxan-2-yl(difluoro)methyl]-2-pyridyl]piperazin-1-yl]sulfonyl-1-oxo-3a,4-dihydro-3H-oxazolo[4,3-c][1,4]benzoxazin-3-yl]methyl]carbamate

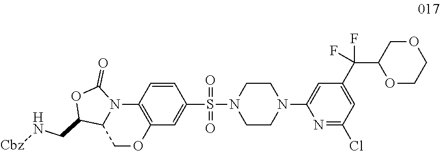

Compounds Int-10 (300 mg, 597 μmol) and Int-18 (230 mg, 810 μmol) were dissolved in the DMSO (6 mL), followed by adding DIPEA (424 μl, 2.43 mmol). The reaction was stirred at 100° C. for 1 day. After the reaction was completed, the reaction was cooled to room temperature and concentrated in vacuo to give crude product, which was purified by silica-gel chromatography (elute with EtOAc:PE=0-90%) to afford compound 017a (120 mg, 19.8% yield) as a white solid. MS obsd. (ESI⁺) [(M+H)⁺]: 750.1.

### Step 2: trans-3-(aminomethyl)-7-[4-[6-chloro-4-[1,4-dioxan-2-yl(difluoro)methyl]-2-pyridyl]piperazin-1-yl]sulfonyl-3a,4-dihydro-3H-oxazolo[4,3-c][1,4]benzoxazin-1-one

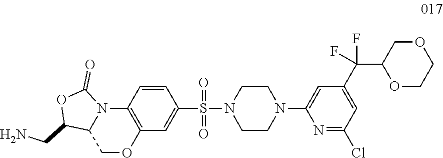 [SMILES: NC[C@H]1OC(=O)N2c3ccc(S(=O)(=O)N4CCN(c5cc(C(F)(F)C6COCCO6)cc(Cl)n5)CC4)cc3OC[C@H]12]

Compound 017a (120 mg, 160 μmol) in TFA (5 mL) was stirred at 70° C. for 4 h. After the reaction was completed, the reaction was concentrated in vacuo to give a crude residue, which was purified by prep-HPLC (HCl as additive) to give Example 017 (45 mg, 37.0% yield) as a white solid. MS obsd. (ESI⁺) [(M+H)⁺]: 616.2.

Exemple 017: ¹H NMR (400 MHz, DMSO-d₆) δ ppm 8.23-8.33 (m, 3H), 8.10-8.21 (m, 1H), 7.38-7.48 (m, 1H), 7.26-7.34 (m, 1H), 6.82-6.89 (m, 1H), 6.69-6.79 (m, 1H), 4.64-4.77 (m, 2H), 4.13-4.28 (m, 4H), 3.72-3.87 (m, 4H), 3.63-3.72 (m, 4H), 3.31-3.41 (m, 2H), 2.90-3.04 (m, 4H). ¹⁹F NMR (376 MHz, DMSO-d₆) δ ppm −106.25-−100.42 (m, 1F), −116.52-−109.30 (m, 1F).

The following Examples 018 to 024 were prepared in analogy to the procedure described for the preparation of Example 017, replacing 2-chloro-4-methyl-6-(trifluoromethyl)pyrimidine with “RCl”, and Int-8 with “Secondary amine” by the reagent indicated in Table 4 in Step 1.

TABLE 4
Compounds synthesis and characterization

| Example | Compounds Name and | Secondary | |
|---|---|---|---|
| No. | Structure | amine and RCl | NMR and (ESI⁺) |
| 018 | trans-3-(aminomethyl)-7-[4- | Secondary | ¹H NMR (400 MHz, |
| | [6-chloro-4-[difluoro(3- | amine: Int-10 | DMSO-d6) δ ppm |
| | oxabicyclo[3.1.0]hexan-6- | RCl: Int-19 | 7.97-8.14 (m, 1H), |
| | yl)methyl]-2- | | 7.28-7.39 (m, 1H), |
| | pyridyl]piperazin-1- | | 7.16-7.26 (m, 1H), |
| | yl]sulfonyl-3a,4-dihydro-3H- | | 6.77-6.86 (m, 1H), |
| | oxazolo[4,3- | | 6.67-6.75 (m, 1H), |
| | c][1,4]benzoxazin-1-one | | 4.50-4.64 (m, 1H), |
| | 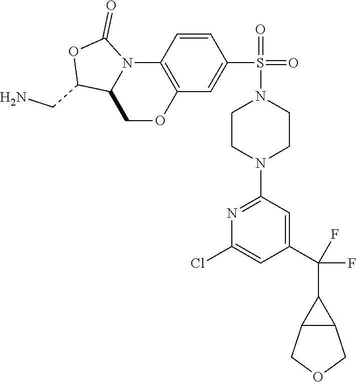 [SMILES: NC[C@@H]1OC(=O)N2c3ccc(S(=O)(=O)N4CCN(c5cc(C(F)(F)C6C7COCC76)cc(Cl)n5)CC4)cc3OC[C@@H]12] | | 4.31-4.43 (m, 1H), 3.97-4.15 (m, 2H), 3.64-3.74 (m, 4H), 3.56-3.64 (m, 4H), 3.47-3.56 (m, 2H), 3.04-3.14 (m, 4H), 2.84-2.93 (m, 4H), 1.82-2.07 (m, 3H). ¹⁹F NMR (376 MHz, DMSO-d₆) δ ppm δ ppm −95.68 (br d, J = 12.26 Hz, 2F). MS obsd. (ESI⁺) [(M + H)⁺]: 612.3. |
| 019 | trans-3-(aminomethyl)-7-[4- | Secondary | ¹H NMR (400 MHz, |
| | [6-chloro-4-[difluoro-(2- | amine: Int-10 | DMSO-d6) δ ppm |
| | methyl-4-pyridyl)methyl]-2- | RCl: Int-20 | 8.47-8.64 (m, 1 H), |
| | pyridyl]piperazin-1- | | 8.00-8.25 (m, 1 H), |
| | yl]sulfonyl-3a,4-dihydro-3H- | | 7.59-7.70 (m, 1 H), |
| | oxazolo[4,3- | | 7.44-7.56 (m, 1 H), |
| | c][1,4]benzoxazin-1-one | | 7.35-7.44 (m, 2 H), |
| | 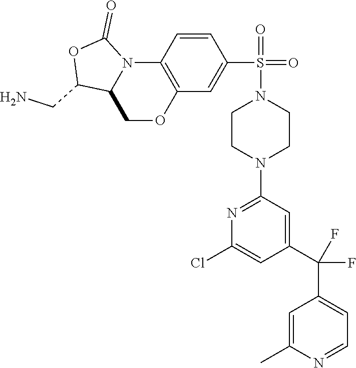 [SMILES: Cc1cc(C(F)(F)c2cc(Cl)nc(N3CCN(S(=O)(=O)c4ccc5c(c4)OC[C@H]4[C@H](CN)OC(=O)N54)CC3)c2)ccn1] | | 7.19-7.35 (m, 1 H), 6.86-7.11 (m, 1 H), 6.75-6.90 (m, 1 H), 4.59-4.69 (m, 1 H), 4.44-4.59 (m, 2 H), 3.99-4.27 (m, 4 H), 3.60-3.71 (m, 4 H), 3.10-3.21 (m, 3 H), 2.88-3.04 (m, 4 H). ¹⁹F NMR (376 MHz, DMSO-d6) δ ppm −95.53 (s, 2F). MS obsd. (ESI⁺) [(M + H)⁺]: 621.4. |
| 020 | cis-3-(aminomethyl)-7-[4-[6- | Secondary | ¹H NMR (400 MHz, |
| | chloro-4-[difluoro-(1-methyl- | amine: Int-9 | DMSO-d6) δ ppm 8.25- |
| | 3,6-dihydro-2H-pyridin-4- | RCl: Int-21 | 8.44 (m, 2 H), 8.13- |
| | yl)methyl]-2- | | 8.24 (m, 2 H), 7.40- |
| | pyridyl]piperazin-1- | | 7.49 (m, 1 H), 7.27- |
| | yl]sulfonyl-3a,4-dihydro-3H- | | 7.38 (m, 1 H), 6.79- |
| | oxazolo[4,3- | | 6.92 (m, 1 H), 6.64- |
| | c][1,4]benzoxazin-1-one | | 6.72 (m, 1 H), 6.10- |
| | 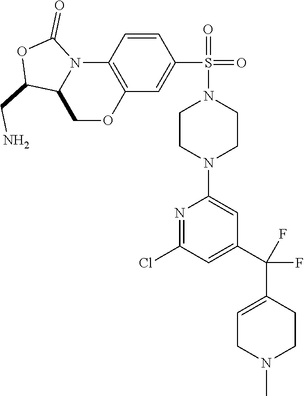 [SMILES: CN1CC=C(C(F)(F)c2cc(Cl)nc(N3CCN(S(=O)(=O)c4ccc5c(c4)OC[C@H]4[C@@H](CN)OC(=O)N54)CC3)c2)CC1] | | 6.24 (m, 1 H), 5.00- 5.15 (m, 1 H), 4.62- 4.73 (m, 1 H), 4.43- 4.58 (m, 1 H), 4.06- 4.21 (m, 1 H), 3.62- 3.72 (m, 5 H), 3.09- 3.22 (m, 2 H), 2.93- 3.03 (m, 4 H), 2.76- 2.83 (m, 3 H), 2.26- 2.40 (m, 2 H), 1.09- 1.36 (m, 2 H). ¹⁹F NMR (376 MHz, DMSO-d6) δ ppm −107.36-−93.48 (m, 2F). MS obsd. (ESI⁺) [(M + H)⁺]: 625.1 |
| 021 | (3R,3aS)-3-(Aminomethyl)-7- | Secondary | ¹H NMR (400 MHz, |
| | [4-[6-chloro-4-[[(2S)-1, 4- | amine: Int-6 | CD₃OD) δ ppm 8.18- |
| | dioxan-2-yl]-difluoro- | RCl: Int-18-A | 8.32 (m, 1H), 7.41- |
| | methyl]-2-pyridyl]piperazin- | | 7.49 (m, 1H), 7.31- |
| | 1-yl]sulfonyl-3a,4-dihydro- | | 7.39 (m, 1H), 6.78- |
| | 3H-oxazolo[4,3- | | 6.83 (m, 1H), 6.70- |
| | c][1,4]benzoxazin-1-one | | 6.74 (m, 1H), 5.05- |
| | 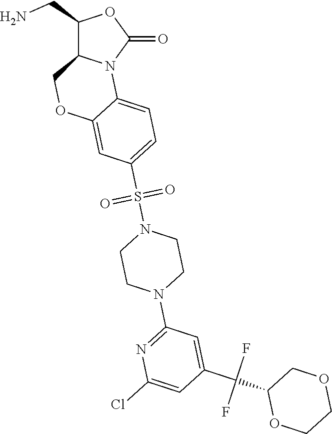 [SMILES: NC[C@H]1OC(=O)N2c3ccc(S(=O)(=O)N4CCN(c5cc(C(F)(F)[C@@H]6COCCO6)cc(Cl)n5)CC4)cc3OC[C@@H]12] | | 5.22 (m, 1H), 4.62- 4.71 (m, 1H), 4.44- 4.60 (m, 1H), 4.04- 4.19 (m, 2H), 3.73- 3.90 (m, 2H), 3.66- 3.73 (m, 6H), 3.58- 3.66 (m, 2H), 3.44- 3.57 (m, 2H), 3.24- 3.40 (m, 2H), 2.96- 3.09 (m, 4H). ¹⁹F NMR (376 MHz, CD₃OD) δ ppm −108.19-−103.19 (m, 1F), −117.35-−112.35 (m, 1F). MS obsd. (ESI⁺) [(M + H)⁺]: 616.6. |
| 022 | (3S,3aR)-3-(aminomethyl)-7- | Secondary | ¹H NMR (400 MHz, |
| | [4-[6-chloro-4-[[(2S)-1,4- | amine: Int-6 | CD₃OD) δ ppm 8.24- |
| | dioxan-2-yl]-difluoro- | RCl: Int-18-B | 8.33 (m, 1H), 7.43- |
| | methyl]-2-pyridyl]piperazin- | | 7.50 (m, 1H), 7.38- |
| | 1-yl]sulfonyl-3a,4-dihydro- | | 7.43 (m, 1H), 6.69- |
| | 3H-oxazolo[4,3- | | 6.78 (m, 2H), 5.09- |
| | c][1,4]benzoxazin-1-one | | 5.23 (m, 1H), 4.61- |
| | 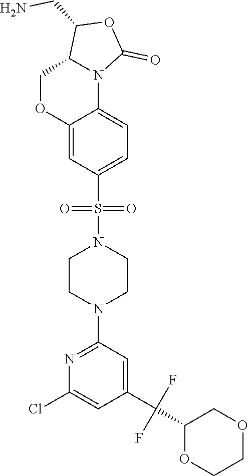 [SMILES: NC[C@@H]1OC(=O)N2c3ccc(S(=O)(=O)N4CCN(c5cc(C(F)(F)[C@@H]6COCCO6)cc(Cl)n5)CC4)cc3OC[C@H]12] | | 4.72 (m, 1H), 4.46- 4.61 (m, 1H), 4.27- 4.42 (m, 1H), 3.99- 4.27 (m, 3H), 3.85- 3.96 (m, 1H), 3.63- 3.83 (m, 8H), 3.48- 3.61 (m, 2H), 3.01- 3.19 (m, 5H), 1.96- 2.11 (m, 1H). ¹⁹F NMR (376 MHz, CD₃OD) δ ppm −109.85-−101.25 (m, 1F), −121.79-−112.08 (m, 1F). MS obsd. (ESI⁺) [(M + H)⁺]: 616.9. |
| 023 | (3S,3aS)-3-(aminomethyl)-7- | Secondary | 1H NMR (400 MHz, |
| | [4-[6-chloro-4-[difluoro- | amine: Int-10 | DMSO-d6) δ ppm 8.85- |
| | [(2R)-morpholin-2- | RCl: Int-23 | 9.41 (m, 1 H), 8.17- |
| | yl]methyl]-2- | | 8.48 (m, 2 H), 8.10- |
| | pyridyl]piperazin-1- | | 8.19 (m, 2 H), 7.39- |
| | yl]sulfonyl-3a,4-dihydro-3H- | | 7.49 (m, 1 H), 7.26- |
| | oxazolo[4,3- | | 7.35 (m, 1 H), 6.82- |
| | c][1,4]benzoxazin-1-one | | 6.90 (m, 1 H), 6.70- |
| | 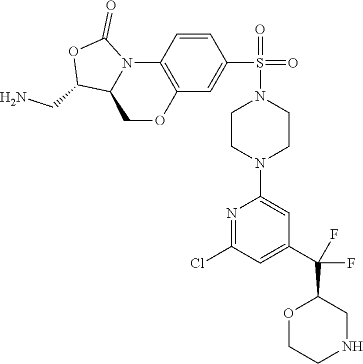 [SMILES: NC[C@@H]1OC(=O)N2c3ccc(S(=O)(=O)N4CCN(c5cc(C(F)(F)[C@H]6CNCCO6)cc(Cl)n5)CC4)cc3OC[C@@H]12] | | 6.82 (m, 1 H), 4.60- 4.80 (m, 2 H), 4.26- 4.43 (m, 1 H), 4.10- 4.23 (m, 2 H), 3.95- 4.10 (m, 1 H), 3.59- 3.78 (m, 6 H), 3.11- 3.26 (m, 1 H), 2.87- 3.11 (m, 7 H). ¹⁹F NMR (376 MHz, DMSO-d6) δ ppm −108.47-−100.69 (m, 1 F), −115.13-−108.74 (m, 1 F). MS obsd. (ESI⁺) [(M + H)+]: 615.2. |
| 024 | (3R,3aR)-3-(aminomethyl)-7- | Secondary | 1H NMR (400 MHz, |
| | [4-[6-chloro-4-[difluoro- | amine: Int-10 | DMSO-d6) δ ppm 8.83- |
| | [(2R)-morpholin-2- | RCl: Int-24 | 9.32 (m, 1 H), 8.04- |
| | yl]methyl]-2- | | 8.46 (m, 3 H), 7.38- |
| | pyridyl]piperazin-1- | | 7.54 (m, 1 H), 7.24- |
| | yl]sulfonyl-3a,4-dihydro-3H- | | 7.38 (m, 1 H), 6.81- |
| | oxazolo[4,3- | | 6.97 (m, 1 H), 6.66- |
| | c][1,4]benzoxazin-1-one | | 6.81 (m, 1 H), 4.56- |
| | 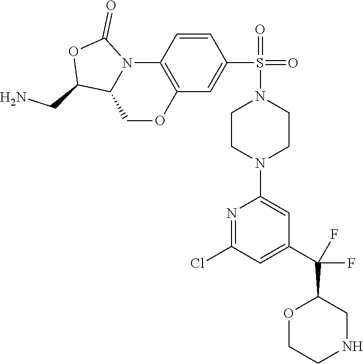 [SMILES: NC[C@H]1OC(=O)N2c3ccc(S(=O)(=O)N4CCN(c5cc(C(F)(F)[C@H]6CNCCO6)cc(Cl)n5)CC4)cc3OC[C@H]12] | | 4.81 (m, 2 H), 4.24- 4.45 (m, 1 H), 4.09- 4.24 (m, 2 H), 3.97- 4.09 (m, 1 H), 3.57- 3.81 (m, 4 H), 3.41- 3.49 (m, 2 H), 3.11- 3.24 (m, 1 H), 2.92- 3.11 (m, 5 H). ¹⁹F NMR (376 MHz, DMSO-d6) δ ppm −108.74-−102.08 (m, 1 F), −115.13-−108.47 (m, 1 F). MS obsd. (ESI⁺) [(M + H)⁺]: 615.2. |

### Example 025: (3R,3aS)-3-[(3-aminoazetidin-1-yl)methyl]-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one

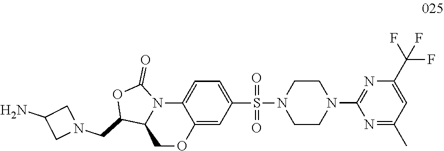 [SMILES: Cc1cc(C(F)(F)F)nc(N2CCN(S(=O)(=O)c3ccc4c(c3)OC[C@H]3[C@@H](CN5CC(N)C5)OC(=O)N43)CC2)n1]

### Step 1. (3S,3aS)-3-(hydroxymethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one

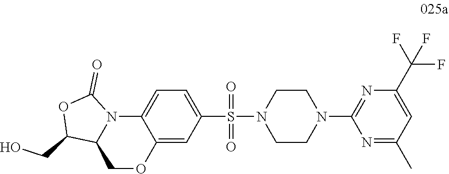 [SMILES: Cc1cc(C(F)(F)F)nc(N2CCN(S(=O)(=O)c3ccc4c(c3)OC[C@H]3[C@@H](CO)OC(=O)N43)CC2)n1]

A mixture of Int-2A (135 mg, 0.365 mmol), 2-chloro-4-methyl-6-(trifluoromethyl)pyrimidine (71.84 mg, 0.365 mmol) and DIPEA (141.7 mg, 191.49 μL, 1.1 mmol) in N,N-dimethylformamide (1.5 mL) was stirred at 100° C. for 16 h. After the reaction was completed, the mixture was concentrated in vacuo to give crude compound 025a (181 mg, 93%). MS (ESI⁺) obsd. [(M+H)⁺]: 530.1.

### Step 2. [(3S,3aS)-7-[4-[4-methyl-6-(trifluoromethyl)pyrimidin-2-yl]piperazin-1-yl]sulfonyl-1-oxo-3a,4-dihydro-3H-oxazolo[4,3-c][1,4]benzoxazin-3-yl]methyl trifluoromethanesulfonate

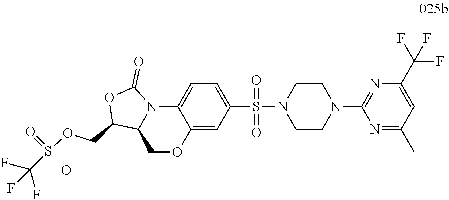

To a mixture of compound 025a (193 mg, 0.365 mmol) and DIPEA (141.32 mg, 1.09 mmol) in dichloromethane (2 mL) at 0° C. was added trifluoromethanesulfonic anhydride (308.52 mg, 1.09 mmol). The resulting mixture was allowed to room temperature and stirred for 2 h. After the reaction was completed, the reaction was quenched with cold water and extracted with DCM. The organic phase was dried with anhydrous sodium sulfate and filtered. The filtrate was concentrated in vacuo and isolated by silica-gel chromatography (elute with EtOAc:PE=0-50%) to give compound 025b (220 mg, 91%). MS (ESI⁺) obsd. [(M+H)⁺]: 662.1.

### Step 3. tert-Butyl N-[1-[[(3R,3aS)-7-[4-[4-methyl-6-(trifluoromethyl)pyrimidin-2-yl]piperazin-1-yl]sulfonyl-1-oxo-3a,4-dihydro-3H-oxazolo[4,3-c][1,4]benzoxazin-3-yl]methyl]azetidin-3-yl]carbamate

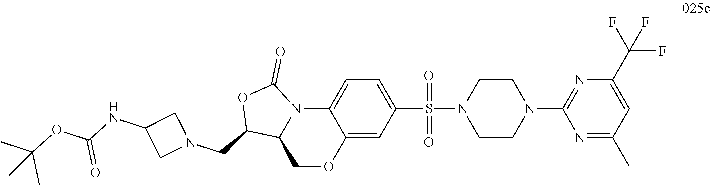 [SMILES: Cc1cc(C(F)(F)F)nc(N2CCN(S(=O)(=O)c3ccc4c(c3)OC[C@H]3[C@@H](CN5CC(NC(=O)OC(C)(C)C)C5)OC(=O)N43)CC2)n1]

A mixture of compound 025b (110 mg, 0.166 mmol), N-(azetidin-3-yl) carbamic acid tert-butyl ester (42.95 mg, 0.249 mmol) and DIPEA (53.72 mg, 0.416 mmol, 2.5 eq) in tetrahydrofuran (2 mL) was heated at 50° C. for 16 h. After the reaction was completed, the mixture was concentrated in vacuo to give crude compound 025c (113 mg, 99% yield), which was used directly without further purification. MS (ESI⁺) obsd. [(M+H)⁺]: 684.2.

### Step 4. (3R,3aS)-3-[(3-aminoazetidin-1-yl)methyl]-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one

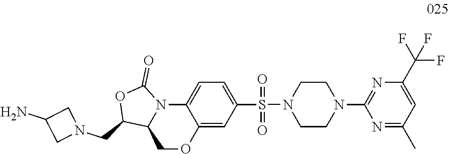 [SMILES: Cc1cc(C(F)(F)F)nc(N2CCN(S(=O)(=O)c3ccc4c(c3)OC[C@H]3[C@@H](CN5CC(N)C5)OC(=O)N43)CC2)n1]

A mixture of compound 025c (113 mg, 0.165 mmol) and TFA (282.67 mg, 2.48 mmol) in dichloromethane (2 mL) was stirred at rt for 1 h. After the reaction was completed, the mixture was concentrated in vacuo to give crude residue, which was purified by prep-HPLC to give Example 025 (49 mg, 48% yield). MS observed (M+H): 584.2.

Example 025: ¹H NMR (400 MHz, DMSO-d₆) δ ppm 8.09 (d, J=8.6 Hz, 1H), 7.30 (dd, J=2.1, 8.6 Hz, 1H), 7.20 (d, J=2.0 Hz, 1H), 6.90 (s, 1H), 4.73 (td, J=5.3, 8.9 Hz, 1H), 4.56 (dd, J=2.9, 10.4 Hz, 1H), 4.27 (m, 1H), 4.13 (m, 1H), 3.80 (m, 4H), 3.38 (m, 3H), 2.90 (br t, J=4.7 Hz, 4H), 2.74 (m, 2H), 2.66 (m, 1H), 2.57 (m, 1H), 2.31 (s, 3H). ¹⁹F NMR (376 MHz, CD₃OD) δ ppm −74.45 (s, 2F).

The following Example 026 was prepared in analogy to the procedure described for the preparation of Example 025, replacing N-(azetidin-3-yl) carbamic acid tert-butyl ester with “Secondary amine by the reagent indicated in Table 5 in Step 3.

TABLE 5
Compounds synthesis and characterization

| Example | Compounds Name and | Secondary | |
|---|---|---|---|
| No. | Structure | amine | NMR and (ESI⁺) |
| 026 | (3R,3aS)-3-[[3- (aminomethyl)azetidin-1- yl]methyl]-7-[4-[4-methyl-6- (trifluoromethyl)pyrimidin- 2-yl]piperazin-1-yl]sulfonyl- 3a,4-dihydro-3H-oxazolo[4,3- c][1,4]benzoxazin-1-one 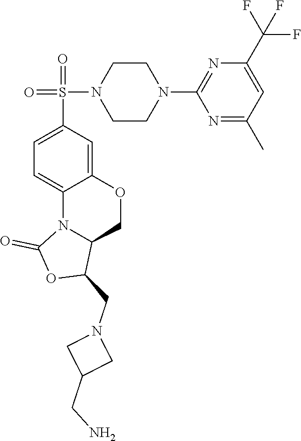 [SMILES: Cc1cc(C(F)(F)F)nc(N2CCN(S(=O)(=O)c3ccc4c(c3)OC[C@H]3[C@@H](CN5CC(CN)C5)OC(=O)N43)CC2)n1] | tert-butyl N- (azetidin-3- ylmethyl) carbamate | ¹H NMR (400 MHz, DMSO-d6) δ ppm 8.17 (d, J = 8.6 Hz, 1H), 7.39 (dd, J = 2.1, 8.7 Hz, 1H), 7.29 (d, J = 2.0 Hz, 1H), 6.98 (s, 1H), 4.81 (td, J = 5.4, 8.8 Hz, 1H), 4.65 (dd, J = 3.0, 10.5 Hz, 1H), 4.36 (m, 1H), 4.19 (br t, J = 10.3 Hz, 1H), 3.88 (m, 4H), 3.31 (q, J = 7.5 Hz, 3H), 2.98 (m, 6H), 2.87 (m, 2H), 2.74 (dd, J = 4.8, 13.3 Hz, 1H), 2.64 (dd, J = 5.9, 13.3 Hz, 1H), 2.39 (s, 3H). ¹⁹F NMR (376 MHz, CD₃OD) δ ppm −74.43 (s, 2F). MS obsd. (ESI⁺) [(M + H)⁺]: 598.2. |

### Example 027: 7-[4-[6-Chloro-4-[difluoro-[rac-(2S)-1,4-dioxan-2-yl]methyl]-2-pyridyl]piperazin-1-yl]sulfonyl-3-(4-piperidylamino)-2,3,3a,4-tetrahydropyrrolo[2,1-c][1,4]benzoxazin-1-one

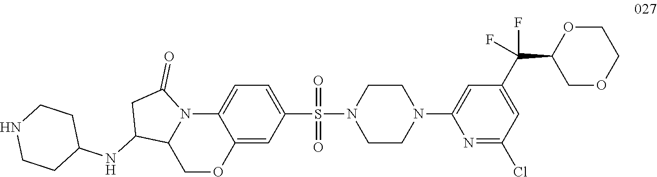 [SMILES: O=C1CC(NC2CCNCC2)C2COc3cc(S(=O)(=O)N4CCN(c5cc(C(F)(F)[C@@H]6COCCO6)cc(Cl)n5)CC4)ccc3N12]

### Step 1. tert-Butyl N-[7-[4-[6-chloro-4-[[(2S)-1,4-dioxan-2-yl]-difluoro-methyl]-2-pyridyl]piperazin-1-yl]sulfonyl-1-oxo-2,3,3a,4-tetrahydropyrrolo[2,1-c][1,4]benzoxazin-3-yl]carbamate

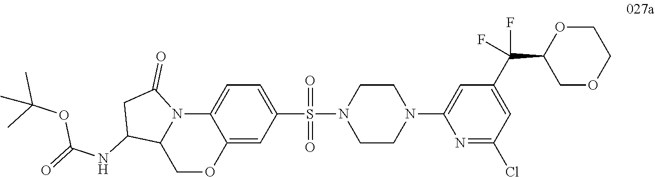 [SMILES: CC(C)(C)OC(=O)NC1CC(=O)N2c3ccc(S(=O)(=O)N4CCN(c5cc(C(F)(F)[C@@H]6COCCO6)cc(Cl)n5)CC4)cc3OCC12]

A mixture of Int-13 (60 mg, 0.133 mmol), Int-18-B (37.67 mg, 0.133 mmol) and DIPEA (17.14 mg, 0.133 mmol) in N, N-dimethylformamide (1.5 mL) was stirred at 100° C. for 16 h. After the reaction was completed, the mixture was diluted with EtOAc (20 mL) and washed with water (10 mL×2). The organic phase was concentrated in vacuo to give crude compound 023a (92 mg, 94%), which was used directly in next step. MS obsd. (ESI⁺) [(M+H)⁺]: 700.2.

### Step 2. 3-Amino-7-[4-[6-chloro-4-[[(2S)-1,4-dioxan-2-yl]-difluoro-methyl]-2-pyridyl]piperazin-1-yl]sulfonyl-2,3,3a,4-tetrahydropyrrolo[2,1-c][1,4]benzoxazin-1-one

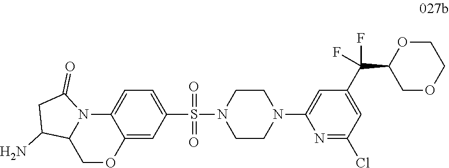 [SMILES: NC1CC(=O)N2c3ccc(S(=O)(=O)N4CCN(c5cc(C(F)(F)[C@@H]6COCCO6)cc(Cl)n5)CC4)cc3OCC12]

A mixture of compound 027a (93 mg, 0.133 mmol) and 2,2,2-trifluoroacetic acid (302.9 mg, 2.66 mmol) in dichloromethane (1 mL) was stirred at room temperature for 1 h. After the reaction was completed, the mixture was concentrated in vacuo to give crude compound 027b (80 m, 100%), which was used directly in next step. MS obsd. (ESI⁺) [(M+H)⁺]: 600.2.

### Step 3. tert-Butyl 4-[[7-[4-[6-chloro-4-[[(2S)-1,4-dioxan-2-yl]-difluoro-methyl]-2-pyridyl]piperazin-1-yl]sulfonyl-1-oxo-2,3,3a,4-tetrahydropyrrolo[2,1-c][1,4]benzoxazin-3-yl]amino]piperidine-1-carboxylate

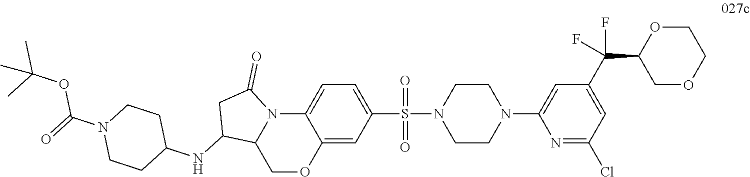 [SMILES: CC(C)(C)OC(=O)N1CCC(NC2CC(=O)N3c4ccc(S(=O)(=O)N5CCN(c6cc(C(F)(F)[C@@H]7COCCO7)cc(Cl)n6)CC5)cc4OCC23)CC1]

A mixture of compound 027b (80 mg, 0.133 mmol) and 4-ketopiperidine-1-carboxylic acid tert-butyl ester in THF (2 mL) in was heated at 80° C. for 2 h. After the reaction was completed, the mixture was quenched with water and extracted with EtOAc (20 mL). The organic layer was dried over anhydrous sodium sulfate and filtered. The filtrate was concentrated in vacuo to give crude compound 027c (104 mg, 100%), which was used directly in next step. MS obsd. (ESI⁺) [(M+H)⁺]: 783.3.

### Step 4. 7-[4-[6-Chloro-4-[[(2S)-1,4-dioxan-2-yl]-difluoro-methyl]-2-pyridyl]piperazin-1-yl]sulfonyl-3-(4-piperidylamino)-2,3,3a,4-tetrahydropyrrolo[2,1-c][1,4]benzoxazin-1-one

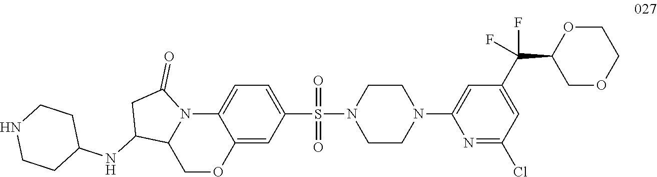 [SMILES: O=C1CC(NC2CCNCC2)C2COc3cc(S(=O)(=O)N4CCN(c5cc(C(F)(F)[C@@H]6COCCO6)cc(Cl)n5)CC4)ccc3N12]

A mixture of compound 027c (104 mg, 0.133 mmol) and 2,2,2-trifluoroacetic acid (302.78 mg, 2.66 mmol) in dichloromethane (2 mL) was stirred at rt for 2 h. After the reaction was completed, the mixture was concentrated in vacuo to give a residue, which was purified by prep-HPLC (HCl as additive) to give Example 027 (21 mg, 22.0% yield). MS obsd. (ESI⁺) [(M+H)⁺]: 683.4.

Example 027: ¹H NMR (400 MHz, DMSO-d₆) δ ppm 8.61 (d, J=8.7 Hz, 1H), 8.39 (br s, 1H), 7.35 (dd, J=2.2, 8.7 Hz, 1H), 7.26 (d, J=2.1 Hz, 1H), 6.84 (s, 1H), 6.75 (s, 1H), 4.70 (dd, J=3.1, 10.6 Hz, 1H), 4.20 (m, 1H), 3.91 (m, 1H), 3.81 (m, 1H), 3.73 (m, 2H), 3.65 (m, 6H), 3.60 (br d, J=2.2 Hz, 1H), 3.10 (m, 4H), 2.97 (m, 4H), 2.70 (br dd, J=8.1, 16.4 Hz, 4H), 2.45 (m, 1H), 1.85 (m, 2H), 1.29 (m, 2H). ¹⁹F NMR (376 MHz, CD₃OD) δ ppm −109.35, (d, 1F), −117.53, (d, 1F).

### Example 028: (3R,3aR)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one

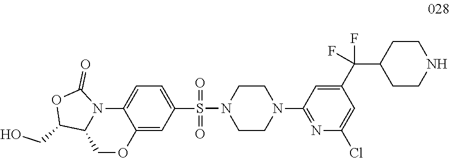 [SMILES: O=C1O[C@@H](CO)[C@H]2COc3cc(S(=O)(=O)N4CCN(c5cc(C(F)(F)C6CCNCC6)cc(Cl)n5)CC4)ccc3N12]

### Step 1: tert-Butyl 4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]piperidine-1-carboxylate

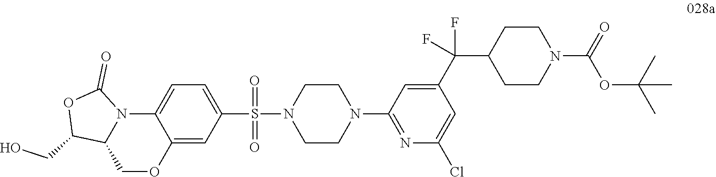 [SMILES: CC(C)(C)OC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of Int-2B (84.6 mg, 222 μmol), Int-22 and DIPEA (86 mg, 666 μmol) in NMP (1.5 mL) was heated at 100° C. overnight. After the reaction was completed, the mixture was diluted with EtOAc (20 mL), washed with sat. aqueous ammonium chloride solution (10 mL), and brine (20 mL). The organic was dried with anhydrous sodium sulfate, filtered and concentrated in vacuo to give crude compound 028a (158 mg, 100%) as light yellow solid. MS obsd. (ESI⁺) [(M+H)]⁺: 714.8.

### Step 2. (3R,3aR)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one

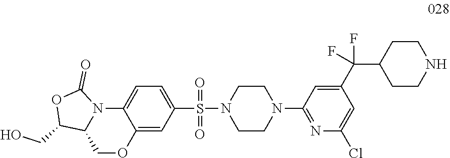 [SMILES: O=C1O[C@@H](CO)[C@H]2COc3cc(S(=O)(=O)N4CCN(c5cc(C(F)(F)C6CCNCC6)cc(Cl)n5)CC4)ccc3N12]

A mixture of compound 028a (0.159 g, 223 μmol) and TFA (862 mg, 6.68 mmol) in DCM (1.5 mL) was stirred at 25° C. for 1 h. After the reaction was completed, the mixture was concentrated in vacuo to give a residue, which was purified by prep-HPLC (HCl as additive) to give Example 028 (57 mg, 39.6% yield) as a white solid. MS obsd. (ESI⁺) [(M+H)]⁺: 614.1.

Example 028: ¹H NMR (400 MHz, DMSO-d₆) δ ppm 8.55 (br d, J=10.5 Hz, 1H), 8.23 (d, J=8.7 Hz, 1H), 7.40 (dd, J=2.1, 8.6 Hz, 1H), 7.30 (d, J=2.1 Hz, 1H), 6.79 (s, 1H), 6.72 (s, 1H), 5.31 (br s, 1H), 4.85 (m, 1H), 4.69 (dd, J=3.1, 10.6 Hz, 1H), 4.44 (m, 1H), 4.23 (t, J=10.3 Hz, 1H), 3.68 (m, 6H), 3.59 (m, 3H), 2.99 (m, 4H), 2.82 (m, 2H), 1.75 (br d, J=12.1 Hz, 2H), 1.51 (m, 2H).

The following Examples 029 to 050 were prepared in analogy to the procedure described for the preparation of Example 028, replacing Int-2B with “Secondary amine”, and Int-22 with “ArCl” by the reagent indicated in Table 6 in Step 1.

TABLE 6
Compounds synthesis and characterization

| Example | Compounds Name and | Secondary | |
|---|---|---|---|
| No. | Structure | amine and ArCl | NMR and (ESI⁺) |
| 029 | (3S,3aS)-7-[4-[6-chloro-4- [difluoro(4- piperidyl)methyl]-2- pyridyl]piperazin-1- yl]sulfonyl-3- (hydroxymethyl)-3a,4- dihydro-3H-oxazolo[4,3- c][1,4]benzoxazin-1-one 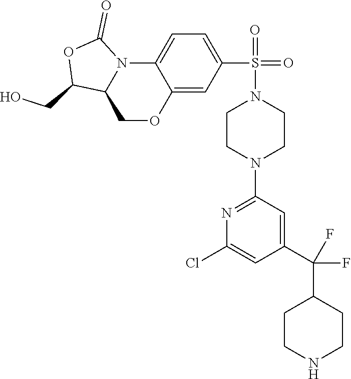 [SMILES: O=C1O[C@H](CO)[C@@H]2COc3cc(S(=O)(=O)N4CCN(c5cc(C(F)(F)C6CCNCC6)cc(Cl)n5)CC4)ccc3N12] | Secondary amine: Int-2A ArCl: Int-22 | ¹H NMR (400 MHz, DMSO-d6) δ ppm 8.22 (m, 1H), 7.39 (dd, J = 2.0, 8.6 Hz, 1H), 7.29 (d, J = 2.1 Hz, 1H), 6.79 (s, 1H), 6.71 (s, 1H), 5.31 (br s, 1H), 4.84 (m, 1H), 4.69 (dd, J = 3.1, 10.5 Hz, 1H), 4.44 (m, 1H), 4.23 (t, J = 10.3 Hz, 1H), 3.67 (m, 5H), 3.58 (br d, J = 8.7 Hz, 1H), 3.29 (m, 1H), 2.99 (m, 4H), 2.82 (m, 2H), 2.57 (m, 1H), 1.74 (br d, J = 12.3 Hz, 2H), 1.50 (m, 2H). ¹⁹F NMR (376 MHz, CD₃OD) δ ppm −109.61 (s, 1F), −109.64 (s, 1F). MS obsd. (ESI⁺) [(M + H)⁺]: 614.1. |
| 030 | 7-[4-[6-Chloro-4-[difluoro- [(2R)-morpholin-2- yl]methyl]-2- pyridyl]piperazin-1- yl]sulfonyl-2,3,3a,4- tetrahydropyrrolo[2,1- c][1,4]benzoxazin-1-one 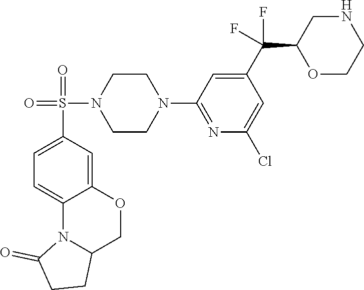 [SMILES: O=C1CCC2COc3cc(S(=O)(=O)N4CCN(c5cc(C(F)(F)[C@H]6CNCCO6)cc(Cl)n5)CC4)ccc3N12] | Secondary amine: Int-12 ArCl: Int-23 | ¹H NMR (400 MHz, DMSO-d6) δ ppm 8.55 (d, J = 8.7 Hz, 1H), 7.27 (dd, J = 2.1, 8.7 Hz, 1H), 7.19 (d, J = 2.1 Hz, 1H), 6.74 (s, 1H), 6.65 (s, 1H), 4.53 (dd, J = 3.2, 10.8 Hz, 1H), 3.98 (ddt, J = 3.2, 6.8, 9.6 Hz, 1H), 3.86 (m, 1H), 3.77 (m, 1H), 3.66 (m, 1H), 3.58 (m, 4H), 2.90 (m, 4H), 2.75 (br d, J = 11.5 Hz, 1H), 2.55 (m, 5H), 2.29 (m, 1H), 2.15 (m, 1H), 1.62 (m, 1H). MS obsd. (ESI⁺) [(M + H)⁺]: 584.1. |
| 031 | 3-Amino-7-[4-[6-chloro-4- [difluoro-[(2R)-morpholin-2- yl]methyl]-2- pyridyl]piperazin-1- yl]sulfonyl-2,3,3a,4- tetrahydropyrrolo[2,1- c][1,4]benzoxazin-1-one 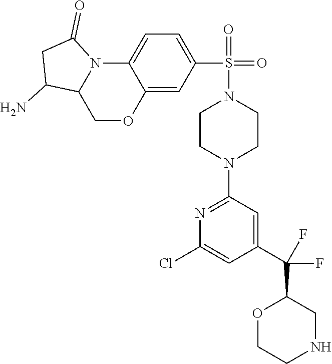 [SMILES: NC1CC(=O)N2c3ccc(S(=O)(=O)N4CCN(c5cc(C(F)(F)[C@H]6CNCCO6)cc(Cl)n5)CC4)cc3OCC12] | Secondary amine: Int-13 ArCl: Int-23 | ¹H NMR (400 MHz, DMSO-d6) δ ppm 8.61 (d, J = 8.7 Hz, 1H), 7.34 (dd, J = 2.1, 8.6 Hz, 1H), 7.26 (d, J = 2.1 Hz, 1H), 6.81 (s, 1H), 6.72 (s, 1H), 4.75 (dd, J = 3.2, 10.8 Hz, 1H), 3.98-3.79 (m, 3H), 3.72 (m, 3H), 3.65 (m, 4H), 2.96 (br t, J = 4.6 Hz, 4H), 2.83 (br d, J = 11.1 Hz, 1H), 2.59 (m, 5H). MS obsd. (ESI⁺) [(M + H)⁺]: 599.0 |
| 032 | cis-7-[4-[6-chloro-4-[difluoro- [(2R)-morpholin-2- yl]methyl]-2- pyridyl]piperazin-1- yl]sulfonyl-3- (hydroxymethyl)-3a,4- dihydro-3H-oxazolo[4,3- c][1,4]benzoxazin-1-one 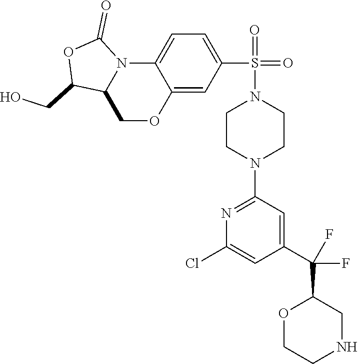 [SMILES: O=C1O[C@H](CO)[C@@H]2COc3cc(S(=O)(=O)N4CCN(c5cc(C(F)(F)[C@H]6CNCCO6)cc(Cl)n5)CC4)ccc3N12] | Secondary amine: Int-1 ArCl: Int-20 | ¹H NMR (400 MHz, DMSO-d6) δ ppm 8.23 (d, J = 8.7 Hz, 1H), 7.39 (dd, J = 2.1, 8.6 Hz, 1H), 7.29 (d, J = 2.1 Hz, 1H), 6.81 (s, 1H), 6.72 (s, 1H), 5.30 (br t, J = 4.9 Hz, 1H), 4.84 (m, 1H), 4.68 (dd, J = 3.2, 10.5 Hz, 1H), 4.44 (m, 1H), 4.23 (m, 1H), 3.92 (m, 1H), 3.77-3.55 (m, 7H), 2.97 (br t, J = 4.7 Hz, 4H), 2.83 (br d, J = 10.8 Hz, 1H), 2.70- 2.52 (m, 3H). MS obsd. (ESI⁺) [(M + H)⁺]: 616.1 |
| 033 | cis-7-[4-[6-Chloro-4- [difluoro(3- piperidyl)methyl]-2- pyridyl]piperazin-1- yl]sulfonyl-3- (hydroxymethyl)-3a,4- dihydro-3H-oxazolo[4,3- c][1,4]benzoxazin-1-one 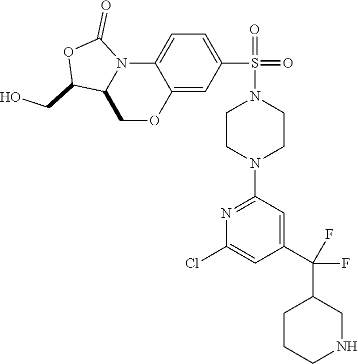 [SMILES: O=C1O[C@H](CO)[C@@H]2COc3cc(S(=O)(=O)N4CCN(c5cc(C(F)(F)C6CCCNC6)cc(Cl)n5)CC4)ccc3N12] | Secondary amine: Int-1 ArCl: Int-25 | ¹H NMR (400 MHz, DMSO-d6) δ ppm 8.23 (d, J = 8.6 Hz, 1H), 7.39 (dd, J = 2.1, 8.7 Hz, 1H), 7.29 (d, J = 2.1 Hz, 1H), 6.79 (s, 1H), 6.68 (s, 1H), 5.31 (br s, 1H), 4.84 (m, 1H), 4.68 (dd, J = 3.2, 10.5 Hz, 1H), 4.44 (m, 1H), 4.23 (t, J = 10.3 Hz, 1H), 3.66 (m, 5H), 3.59 (m, 1H), 2.98 (m, 4H), 2.89 (br d, J = 7.2 Hz, 3H), 2.34 (m, 3H), 1.68-1.59 (m, 2H). MS obsd. (ESI⁺) [(M + H)⁺]: 614.2 |
| 034 | (3R,3aR)-7-[4-[4-[3- azabicyclo[3.1.0]hexan-6- yl(difluoro)methyl]-6-chloro- 2-pyridyl]piperazin-1- yl]sulfonyl-3- (hydroxymethyl)-3a,4- dihydro-3H-oxazolo[4,3- c][1,4]benzoxazin-1-one 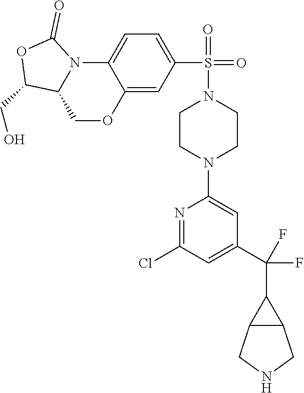 [SMILES: O=C1O[C@@H](CO)[C@H]2COc3cc(S(=O)(=O)N4CCN(c5cc(C(F)(F)C6C7CNCC76)cc(Cl)n5)CC4)ccc3N12] | Secondary amine: Int-2B ArCl: Int-27 | ¹H NMR (400 MHz, DMSO-d6) δ ppm 8.23 (d, J = 8.6 Hz, 1H), 7.39 (dd, J = 2.1, 8.7 Hz, 1H), 7.29 (d, J = 2.1 Hz, 1H), 6.82 (s, 1H), 6.76 (s, 1H), 5.32 (br s, 1H), 4.84 (m, 1H), 4.69 (dd, J = 3.1, 10.5 Hz, 1H), 4.44 (m, 1H), 4.23 (t, J = 10.3 Hz, 1H), 3.68 (m, 5H), 3.58 (m, 1H), 3.34 (m, 4H), 2.99 (br t, J = 4.6 Hz, 4H), 2.19 (br s, 2H), 1.79 (tt, J = 3.6, 12.2 Hz, 1H). ¹⁹F NMR (376 MHz, METHANOL-d₄) δ ppm −101.67 (s, 1F), −101.70 (s, 1F). MS obsd. (ESI⁺) [(M + H)⁺]: 612.2. |
| 035 | (3R,3aR)-7-[4-[4-[azetidin-3- yl(difluoro)methyl]-6-chloro- 2-pyridyl]piperazin-1- yl]sulfonyl-3- (hydroxymethyl)-3a,4- dihydro-3H-oxazolo[4,3- c][1,4]benzoxazin-1-one 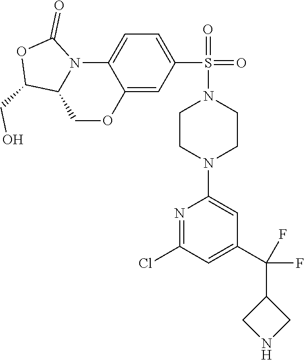 [SMILES: O=C1O[C@@H](CO)[C@H]2COc3cc(S(=O)(=O)N4CCN(c5cc(C(F)(F)C6CNC6)cc(Cl)n5)CC4)ccc3N12] | Secondary amine: Int-2B ArCl: Int-28 | ¹H NMR (400 MHz, DMSO-d6) δ ppm 8.23 (d, J = 8.7 Hz, 1H), 7.39 (dd, J = 2.1, 8.7 Hz, 1H), 7.29 (d, J = 2.1 Hz, 1H), 6.85 (s, 1H), 6.80 (s, 1H), 5.32 (br s, 1H), 4.84 (m, 1H), 4.69 (dd, J = 3.1, 10.6 Hz, 1H), 4.44 (m, 1H), 4.22 (m, 1H), 4.03 (m, 4H), 3.79 (m, 1H), 3.68 (m, 5H), 3.58 (m, 1H), 2.98(m, 4H). ¹⁹F NMR (376 MHz, CD₃OD) δ ppm −110.23 (s, 1F), −110.27 (s, 1F). MS obsd. (ESI⁺) [(M + H)⁺]: 586.2. |
| 036 | (3R,3aR)-7-[4-[4-[3- azabicyclo[3.1.0]hexan-1- yl(difluoro)methyl]-6-chloro- 2-pyridyl]piperazin-1- yl]sulfonyl-3- (hydroxymethyl)-3a,4- dihydro-3H-oxazolo[4,3- c][1,4]benzoxazin-1-one 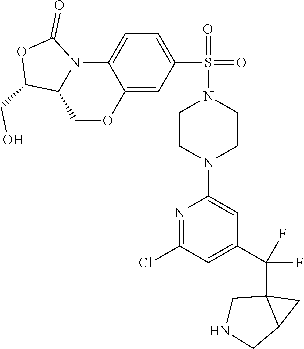 [SMILES: O=C1O[C@@H](CO)[C@H]2COc3cc(S(=O)(=O)N4CCN(c5cc(C(F)(F)C67CNCC6C7)cc(Cl)n5)CC4)ccc3N12] | Secondary amine: Int-2B ArCl: Int-29 | ¹H NMR (400 MHz, DMSO-d6) δ ppm 8.33 (d, J = 8.6 Hz, 1H), 7.49 (dd, J = 2.1, 8.6 Hz, 1H), 7.39 (d, J = 2.1 Hz, 1H), 6.86 (s, 1H), 6.80 (s, 1H), 5.41 (br s, 1H), 4.94 (m, 1H), 4.78 (dd, J = 3.2, 10.5 Hz, 1H), 4.54 (m, 1H), 4.33 (m, 1H), 3.78 (m, 5H), 3.68 (m, 1H), 3.08 (br t, J = 4.6 Hz, 4H), 2.86 (m, 4H), 1.93 (m, 1H), 1.05 (dd, J = 5.2, 8.4 Hz, 1H), 0.89 (m, 1H). ¹⁹F NMR (376 MHz, CD₃OD) δ ppm −100.66 (m, 2F). MS obsd. (ESI⁺) [(M + H)⁺]: 612.1 |
| 037 | (3S,3aR)-7-[4-[6-chloro-4- [difluoro(4- piperidyl)methyl]-2- pyridyl]piperazin-1- yl]sulfonyl-3- (hydroxymethyl)-3a,4- dihydro-3H-oxazolo[4,3- c][1,4]benzoxazin-1-one 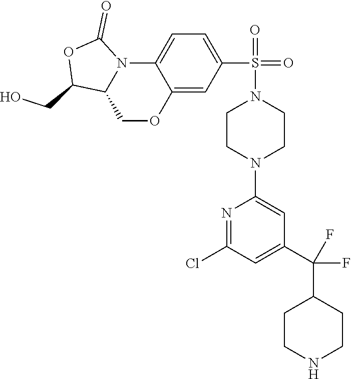 [SMILES: O=C1O[C@H](CO)[C@H]2COc3cc(S(=O)(=O)N4CCN(c5cc(C(F)(F)C6CCNCC6)cc(Cl)n5)CC4)ccc3N12] | Secondary amine: Int-4B ArCl: Int-22 | ¹H NMR (400 MHz, DMSO-d6) δ ppm 8.06 (d, J = 8.6 Hz, 1H), 7.32 (dd, J = 2.1, 8.7 Hz, 1H), 7.22 (d, J = 2.0 Hz, 1H), 6.71 (s, 1H), 6.63 (s, 1H), 4.54 (m, 1H), 4.42 (br d, J = 4.9 Hz, 1H), 4.03 (m, 2H), 3.69 (m, 1H), 3.60 (m, 6H), 3.02 (br d, J = 11.7 Hz, 2H), 2.91 (m, 4H), 2.49 (m, 1H), 2.28 (m, 1H), 1.51 (br d, J = 12.2 Hz, 2H), 1.27 (m, 2H) ¹⁹F NMR (376 MHz, CD₃OD) δ ppm −109.81(d, 2F) MS obsd. (ESI⁺) [(M + H)⁺]: 614.2 |
| 038 | (3R,3aS)-7-[4-[6-chloro-4- [difluoro(4- piperidyl)methyl]-2- pyridyl]piperazin-1- yl]sulfonyl-3- (hydroxymethyl)-3a,4- dihydro-3H-oxazolo[4,3- c][1,4]benzoxazin-1-one 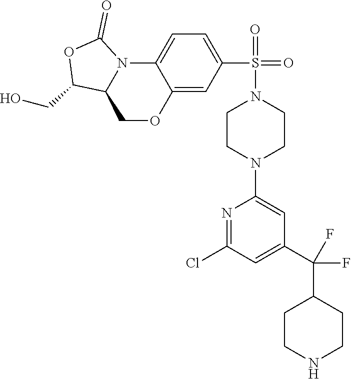 [SMILES: O=C1O[C@@H](CO)[C@@H]2COc3cc(S(=O)(=O)N4CCN(c5cc(C(F)(F)C6CCNCC6)cc(Cl)n5)CC4)ccc3N12] | Secondary amine: Int-4A ArCl: Int-22 | ¹H NMR (400 MHz, DMSO-d6) δ ppm 8.06 (d, J = 8.6 Hz, 1H), 7.32 (dd, J = 2.1, 8.6 Hz, 1H), 7.22 (d, J = 2.0 Hz, 1H), 6.71 (s, 1H), 6.63 (s, 1H), 4.54 (m, 1H), 4.42 (br d, J = 4.6 Hz, 1H), 4.02 (m, 2H), 3.69 (m, 1H), 3.60 (m, 6H), 3.01 (br d, J = 11.6 Hz, 2H), 2.91 (br s, 4H), 2.49 (m, 1H), 2.27 (m, 1H), 1.50 (br d, J = 12.2 Hz, 2H), 1.25 (m, 2H) ¹⁹F NMR (376 MHz, CD₃OD) δ ppm −109.73(d, 2F). MS obsd. (ESI⁺) [(M + H)⁺]: 614.2. |
| 039 | (3R,3aR)-7-[4-[4-[2- azaspiro[3.3]heptan-6- yl(difluoro)methyl]-6-chloro- 2-pyridyl]piperazin-1- yl]sulfonyl-3- (hydroxymethyl)-3a,4- dihydro-3H-oxazolo[4,3- c][1,4]benzoxazin-1-one 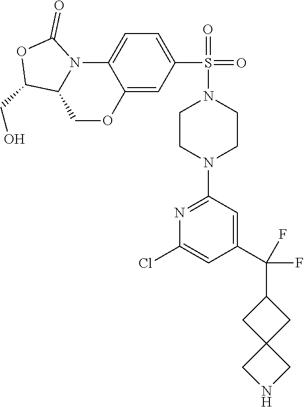 [SMILES: O=C1O[C@@H](CO)[C@H]2COc3cc(S(=O)(=O)N4CCN(c5cc(C(F)(F)C6CC7(CNC7)C6)cc(Cl)n5)CC4)ccc3N12] | Secondary amine: Int-2B ArCl: Int-30 | ¹H NMR (400 MHz, DMSO-d₆) δ ppm 8.16 (d, J = 8.6 Hz, 1H), 7.32 (dd, J = 2.1, 8.7 Hz, 1H), 7.22 (d, J = 2.1 Hz, 1H), 6.71 (s, 1H), 6.65 (s, 1H), 4.77 (m, 1H), 4.62 (dd, J = 3.1, 10.6 Hz, 1H), 4.37 (m, 1H), 4.15 (m, 1H), 3.86 (td, J = 6.1, 17.8 Hz, 4H), 3.64 (m, 1H), 3.59 (m, 4H), 3.51 (m, 1H), 2.90 (m, 5H), 2.19 (m, 4H). ¹⁹F NMR (376 MHz, CD₃OD) δ ppm −110.07(m, 2F). MS obsd. (ESI⁺) [(M + H)⁺]: 626.2 |
| 040 | (3R,3aS)-7-[4-[6-chloro-4- [difluoro-(5- methylmorpholin-2- yl)methyl]-2- pyridyl]piperazin-1- yl]sulfonyl-3- (hydroxymethyl)-3a,4- dihydro-3H-oxazolo[4,3- c][1,4]benzoxazin-1-one 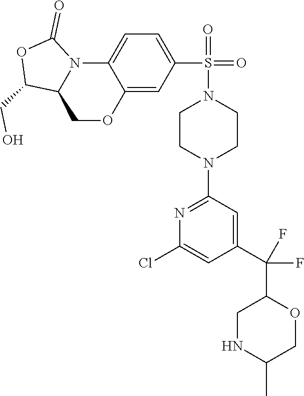 [SMILES: CC1COC(C(F)(F)c2cc(Cl)nc(N3CCN(S(=O)(=O)c4ccc5c(c4)OC[C@H]4[C@H](CO)OC(=O)N54)CC3)c2)CN1] | Secondary amine: Int-4A ArCl: Int-31 | ¹H NMR (400 MHz, DMSO-d₆) δ ppm 8.06 (d, J = 8.5 Hz, 1H), 7.32 (dd, J = 2.0, 8.5 Hz, 1H), 7.22 (d, J = 2.0 Hz, 1H), 6.81 (s, 1H), 6.72 (s, 1H), 5.28 (t, J = 5.5 Hz, 1H), 4.54 (m, 1H), 4.42 (br d, J = 4.5 Hz, 1H), 4.20 (m, 1H), 4.03 (m, 2H), 3.69 (m, 3H), 3.61 (br s, 4H), 3.43 (m, 2H), 3.07 (m, 1H), 2.91 (br s, 4H), 1.19 (d, J = 6.8 Hz, 3H). MS obsd. (ESI⁺) [(M + H)⁺]: 630.2. |
| 041 | (3R,3aR)-7-[4-[4-[1-(2- aminopropanoyl)azetidin-3- yl]-difluoro-methyl]-6- chloro-2-pyridyl]piperazin-1- yl]sulfonyl-3- (hydroxymethyl)-3a,4- dihydro-3H-oxazolo[4,3- c][1,4]benzoxazin-1-one 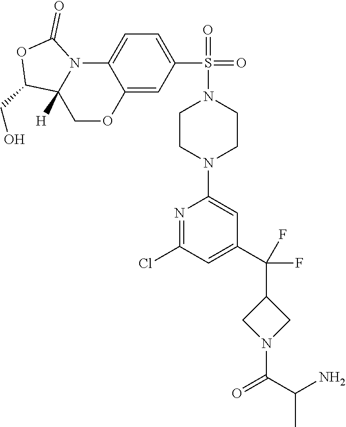 [SMILES: CC(N)C(=O)N1CC(C(F)(F)c2cc(Cl)nc(N3CCN(S(=O)(=O)c4ccc5c(c4)OC[C@@H]4[C@H](CO)OC(=O)N54)CC3)c2)C1] | Secondary amine: Int-2B ArCl: Int-32 | ¹H NMR (400 MHz, DMSO-d6) δ ppm 8.23 (d, J = 8.6 Hz, 1H), 7.39 (dd, J = 2.1, 8.6 Hz, 1H), 7.29 (d, J = 2.1 Hz, 1H), 6.88 (d, J = 9.0 Hz, 1H), 6.82 (d, J = 7.7 Hz, 1H), 4.84 (m, 1H), 4.68 (dd, J = 3.1, 10.5 Hz, 1H), 4.44 (m, 1H), 4.23 (m, 3H), 3.91 (m, 1H), 3.84 (m, 1H), 3.68 (m, 5H), 3.58 (m, 3H), 2.98 (br s, 4H), 1.11 (t, J = 6.4 Hz, 3H). ¹⁹F NMR (376 MHz, CD₃OD) δ ppm −111.12(m, 2F). MS obsd. (ESI⁺) [(M + H)⁺]: 657.2 |
| 042 | (3R,3aR)-7-[4-[4-(4- aminocyclohexoxy)-6-chloro- 2-pyridyl]piperazin-1- yl]sulfonyl-3- (hydroxymethyl)-3a,4- dihydro-3H-oxazolo[4,3- c][1,4]benzoxazin-1-one 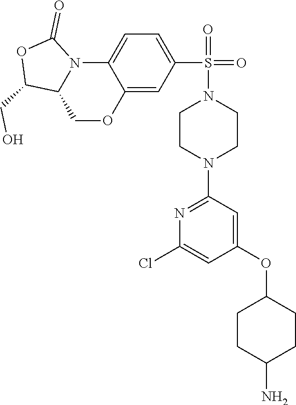 [SMILES: NC1CCC(Oc2cc(Cl)nc(N3CCN(S(=O)(=O)c4ccc5c(c4)OC[C@@H]4[C@H](CO)OC(=O)N54)CC3)c2)CC1] | Secondary amine: Int-2B ArCl: tert-butyl N-[4- [(2,6-dichloro- 4-pyridyl)oxy] cyclohexyl] carbamate | ¹H NMR (400 MHz, DMSO-d₆) δ ppm 8.06 (d, J = 8.5 Hz, 1H), 7.32 (dd, J = 2.0, 8.5 Hz, 1H), 7.22 (d, J = 1.8 Hz, 1H), 6.35 (s, 1H), 6.15 (s, 1H), 5.28 (br s, 1H), 4.54 (m, 1H), 4.38 (m, 2H), 4.04 (m, 2H), 3.70 (br d, J = 12.5 Hz, 1H), 3.61 (br d, J = 12.3 Hz, 1H), 3.49 (m, 4H), 2.96 (br s, 1H), 2.85 (m, 4H), 1.97 (m, 2H), 1.86 (br d, J = 10.3 Hz, 2H), 1.35 (m, 4H). MS obsd. (ESI⁺) [(M + H)⁺]: 594.3 |
| 043 | 3-Amino-7-[4-[4-(4- aminocyclohexoxy)-6-chloro- 2-pyridyl]piperazin-1- yl]sulfonyl-2,3,3a,4- tetrahydropyrrolo[2,1- c][1,4]benzoxazin-1-one 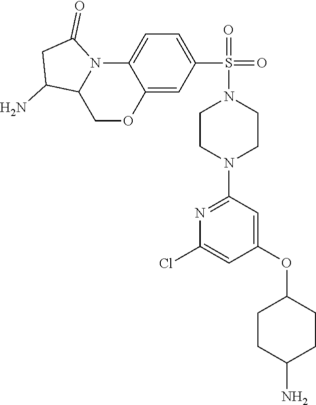 [SMILES: NC1CCC(Oc2cc(Cl)nc(N3CCN(S(=O)(=O)c4ccc5c(c4)OCC4C(N)CC(=O)N54)CC3)c2)CC1] | Secondary amine: Int-13 ArCl: tert-butyl N-[4- [(2,6-dichloro- 4-pyridyl)oxy] cyclohexyl] carbamate | ¹H NMR (500 MHz, DMSO-d₆) δ ppm 8.53 (d, J = 8.7 Hz, 1H), 7.39 (dd, J = 2.1, 8.7 Hz, 1H), 7.30 (d, J = 2.1 Hz, 1H), 6.44 (d, J = 1.4 Hz, 1H), 6.22 (s, 1H), 4.78 (dd, J = 2.6, 10.2 Hz, 1H), 4.43 (m, 1H), 4.09 (m, 2H), 3.97 (m, 1H), 3.80 (m, 1H), 3.55 (m, 4H), 2.94 (br s, 4H), 2.78 (m, 2H), 2.03 (br d, J = 10.7 Hz, 2H), 1.93 (br d, J = 11.0 Hz, 2H), 1.51-1.35 (m, 4H). MS obsd. (ESI⁺) [(M + H)⁺]: 577.1 |
| 044 | (E)-4-[2-[4-[[(3R,3aR)-3- (hydroxymethyl)-1-oxo-3a,4- dihydro-3H-oxazolo[4,3- c][1,4]benzoxazin-7- yl]sulfonyl]piperazin-1-yl]-6- chloro-4-pyridyl]-N-(3- aminopropyl)-4,4-difluoro- but-2-enamide 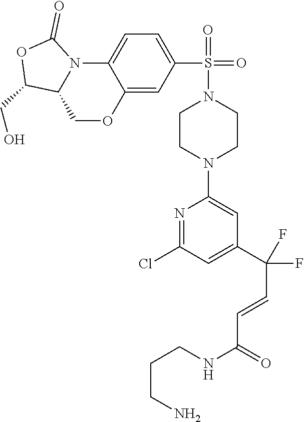 [SMILES: NCCCNC(=O)/C=C/C(F)(F)c1cc(Cl)nc(N2CCN(S(=O)(=O)c3ccc4c(c3)OC[C@@H]3[C@H](CO)OC(=O)N43)CC2)c1] | Secondary amine: Int-2B ArCl: Int-33 | ¹H NMR (400 MHz, DMSO-d6) δ ppm 8.43 (t, J = 5.7 Hz, 1H), 8.16 (d, J = 8.6 Hz, 1H), 7.32 (dd, J = 2.1, 8.6 Hz, 1H), 7.22 (d, J = 2.1 Hz, 1H), 6.83 (m, 1H), 6.78(m, 1H), 6.70 (m, 1H), 6.34 (m, 1H), 5.24 (br s, 1H), 4.77 (m, 1H), 4.61 (dd, J = 3.1, 10.5 Hz, 1H), 4.37 (m, 1H), 4.16 (t, J = 10.3 Hz, 1H), 3.61 (m, 5H), 3.51 (br dd, J = 3.7, 12.4 Hz, 1H), 3.14 (m, 2H), 2.91 (br t, J = 4.6 Hz, 4H), 2.72 (m, 2H), 1.63 (m, 2H). ¹⁹F NMR (376 MHz, CD₃OD) δ ppm −94.96(d, 2F). MS obsd. (ESI⁺) [(M + H)⁺]: 657.1 |
| 045 | 4-[[2-[4-[(3-Amino-1-oxo- 2,3,3a,4- tetrahydropyrrolo[2,1- c][1,4]benzoxazin-7- yl)sulfonyl]piperazin-1-yl]-6- chloro-4-pyridyl]-difluoro- methyl]-N-(3- aminopropyl) cyclohexanecarboxamide 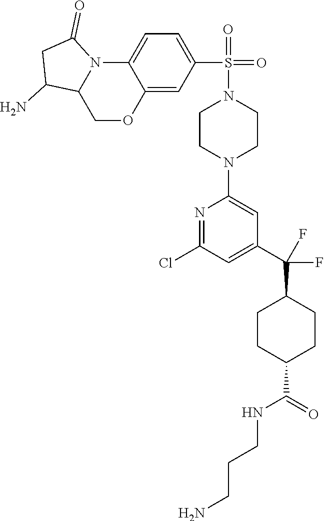 [SMILES: NCCCNC(=O)[C@H]1CC[C@H](C(F)(F)c2cc(Cl)nc(N3CCN(S(=O)(=O)c4ccc5c(c4)OCC4C(N)CC(=O)N54)CC3)c2)CC1] | Secondary amine: Int-13 ArCl: Int-34 | ¹H NMR (400 MHz, DMSO-d₆) δ ppm 8.63 (d, J = 8.7 Hz, 1H), 7.87 (br t, J = 5.5 Hz, 1H), 7.34 (dd, J = 2.1, 8.7 Hz, 1H), 7.25 (d, J = 2.1 Hz, 1H), 6.78 (s, 1H), 6.71 (s, 1H), 4.75 (dd, J = 3.1, 10.8 Hz, 1H), 3.85 (m, 1H), 3.66 (m, 4H), 3.08 (m, 2H), 2.96 (m, 4H), 2.68 (m, 3H), 2.59 (m, 2H), 2.44 (m, 1H), 2.19 (m, 1H), 2.01(m, 1H), 1.73 (m, 2H), 1.63 (m, 4H), 1.33 (m, 2H), 1.11 (m, 2H). ¹⁹F NMR (376 MHz, CD₃OD) δ ppm −109.14(d, 2F). MS obsd. (ESI⁺) [(M + H)⁺]: 696.3. |
| 046 | 4-[[2-[4-[[(3R,3aR)-3- (hydroxymethyl)-1-oxo-3a,4- dihydro-3H-oxazolo[4,3- c][1,4]benzoxazin-7- yl]sulfonyl]piperazin-1-yl]-6- chloro-4-pyridyl]-difluoro- methyl]-N-(3- aminopropyl) cyclohexanecarboxamide 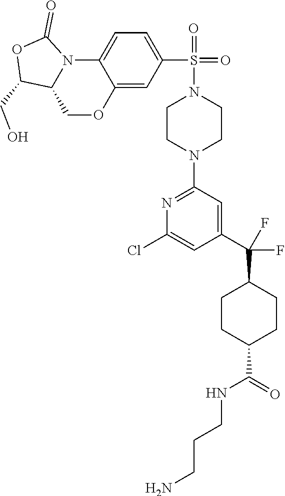 [SMILES: NCCCNC(=O)[C@H]1CC[C@H](C(F)(F)c2cc(Cl)nc(N3CCN(S(=O)(=O)c4ccc5c(c4)OC[C@@H]4[C@H](CO)OC(=O)N54)CC3)c2)CC1] | Secondary amine: Int-2B ArCl: Int-34 | ¹H NMR (400 MHz, DMSO-d₆) δ ppm 8.22 (d, J = 8.5 Hz, 1H), 7.90 (br t, J = 5.8 Hz, 1H), 7.38 (dd, J = 2.0, 8.8 Hz, 1H), 7.28 (d, J = 2.0 Hz, 1H), 6.78 (s, 1H), 6.70 (s, 1H), 5.32 (br s, 1H), 4.83 (m, 1H), 4.68 (dd, J = 3.1, 10.4 Hz, 1H), 4.44 (m, 1H), 4.22 (t, J = 10.3 Hz, 1H), 3.67 (m, 5H), 3.57 (br d, J = 10.5 Hz, 1H), 3.16 (m, 1H), 3.07 (q, J = 6.5 Hz, 2H), 2.97 (br s, 4H), 2.73 (t, J = 7.4 Hz, 2H), 2.18 (m, 1H), 2.02 (m, 1H), 1.75 (m, 2H), 1.63 (m, 3H), 1.33 (m, 2H), 1.13 (m, 2H). MS obsd. (ESI⁺) [(M + H)⁺]: 713.1. |
| 047 | 4-[[2-[4-[[cis-3- (aminomethyl)-1-oxo-3a,4- dihydro-3H-oxazolo[4,3- c][1,4]benzoxazin-7- yl]sulfonyl]piperazin-1-yl]-6- chloro-4-pyridyl]-difluoro- methyl]-N-(3- aminopropyl) cyclohexanecarboxamide 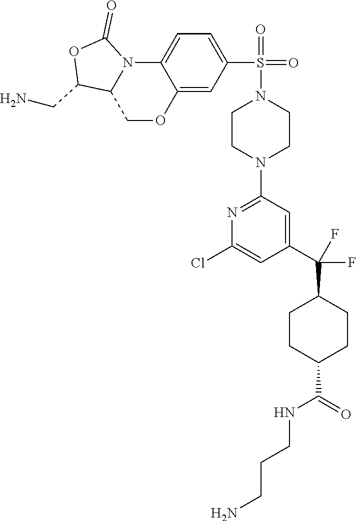 [SMILES: NCCCNC(=O)[C@H]1CC[C@H](C(F)(F)c2cc(Cl)nc(N3CCN(S(=O)(=O)c4ccc5c(c4)OC[C@@H]4[C@H](CN)OC(=O)N54)CC3)c2)CC1] | Secondary amine: Int-9 ArCl: Int-34 | ¹H NMR (400 MHz, DMSO-d₆) δ ppm 8.58 (br s, 3 H) 8.20 (d, J = 8.68 Hz, 1 H) 8.03 (br d, J = 5.01 Hz, 4 H) 7.41 (dd, J = 8.62, 1.90 Hz, 1 H) 7.31 (d, J = 1.96 Hz, 1 H) 6.80 (s, 1 H) 6.70 (s, 1 H) 5.21 (br s, 1 H) 4.70 (br dd, J = 10.03, 2.81 Hz, 1 H) 4.42-4.58 (m, 1 H) 4.15 (br t, J = 10.33 Hz, 1 H) 3.66 (br s, 4 H) 3.12-3.33 (m, 2 H) 3.07 (q, J = 6.32 Hz, 2 H) 2.95 (br s, 4 H) 2.65-2.80 (m, 2 H) 2.10-2.28 (m, 1 H) 2.04 (br t, J = 11.68 Hz, 1 H) 1.59- 1.81 (m, 6 H) 1.32 (q, J = 11.90 Hz, 2 H) 1.11 (q, J = 12.10 Hz, 2 H). ¹⁹F NMR (376 MHz, DMSO-d₆) δ ppm −104.09 (br s, 2 F). MS obsd. (ESI⁺) [(M + H)⁺]: 712.2. |
| 048 | 4-[[2-[4-[[(3R,3aS)-3- (hydroxymethyl)-1-oxo-3a,4- dihydro-3H-oxazolo[4,3- c][1,4]benzoxazin-7- yl]sulfonyl]piperazin-1-yl]-6- chloro-4-pyridyl]-difluoro- methyl]-N,N-bis(3- aminopropyl) cyclohexanecarboxamide 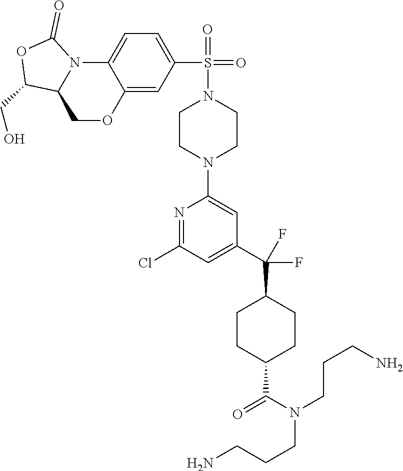 [SMILES: NCCCN(CCCN)C(=O)[C@H]1CC[C@H](C(F)(F)c2cc(Cl)nc(N3CCN(S(=O)(=O)c4ccc5c(c4)OC[C@H]4[C@H](CO)OC(=O)N54)CC3)c2)CC1] | Secondary amine: Int-4A ArCl: Int-35 | ¹H NMR (500 MHz, DMSO-d₆) δ ppm 8.13 (d, J = 8.5 Hz, 1H), 7.39 (dd, J = 2.1, 8.6 Hz, 1H), 7.29 (d, J = 2.0 Hz, 1H), 6.79 (s, 1H), 6.70 (s, 1H), 4.61 (m, 1H), 4.49 (m, 1H), 4.11 (m, 2H), 3.76 (m, 2H), 3.70 (m, 1H), 3.65 (m, 4H), 3.34 (m, 2H), 3.27 (m, 2H), 2.98 (m, 4H), 2.78 (br t, J = 6.6 Hz, 2H), 2.66 (br t, J = 6.9 Hz, 2H), 2.22 (quin, J = 13.0 Hz, 1H), 1.83-1.56 (m, 8H), 1.44-1.32 (m, 2H), 1.30-1.20 (m, 2H). MS obsd. (ESI⁺) [(M + H)⁺]: 770.1. |
| 049 | 4-[[2-[4-[[(3R,3aS)-3- (hydroxymethyl)-1-oxo-3a,4- dihydro-3H-oxazolo[4,3- c][1,4]benzoxazin-7- yl]sulfonyl]piperazin-1-yl]-6- chloro-4-pyridyl]-difluoro- methyl]-N-(3- aminopropyl) cyclohexanecarboxamide 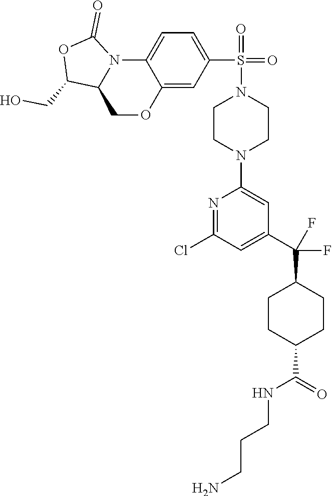 [SMILES: NCCCNC(=O)[C@H]1CC[C@H](C(F)(F)c2cc(Cl)nc(N3CCN(S(=O)(=O)c4ccc5c(c4)OC[C@H]4[C@H](CO)OC(=O)N54)CC3)c2)CC1] | Secondary amine: Int-4A ArCl: Int-34 | 1H NMR (400 MHz, CD₃OD) δ ppm 8.65- 8.44 (m, 1H), 8.26- 8.02 (m, 1H), 7.46- 7.26 (m, 2H), 6.74- 6.52 (m, 2H), 4.67- 4.54 (m, 3H), 4.52- 4.43 (m, 1H), 4.27- 4.13 (m, 1H), 4.10- 3.97 (m, 1H), 3.97- 3.86 (m, 1H), 3.84- 3.74 (m, 1H), 3.73- 3.65 (m, 4H), 3.26- 3.20 (m, 2H), 3.14- 3.02 (m, 4H), 2.93- 2.81 (m, 2H), 2.21- 2.00 (m, 2H), 1.94- 1.67 (m, 6H), 1.52- 1.08 (m, 4H). ¹⁹F NMR (376 MHz, CD₃OD) δ ppm −107.08 (br d, J = 13.63 Hz, 2F). MS obsd. (ESI⁺) [(M + H)⁺]: 713.5. |
| 050 | trans-7-[4-[6-chloro-4- [difluoro-[(2S)-morpholin-2- yl]methyl]-2- pyridyl]piperazin-1- yl]sulfonyl-3- (hydroxymethyl)-3a,4- dihydro-3H-oxazolo[4,3- c][1,4]benzoxazin-1-one 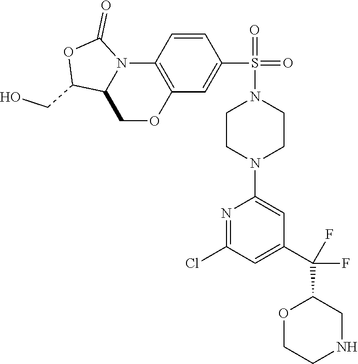 [SMILES: O=C1O[C@@H](CO)[C@@H]2COc3cc(S(=O)(=O)N4CCN(c5cc(C(F)(F)[C@@H]6CNCCO6)cc(Cl)n5)CC4)ccc3N12] | Secondary amine: Int-4 ArCl: Int-24 | H NMR (400 MHz, DMSO-d6) δ ppm 8.19- 8.28 (m, 1 H), 7.35- 7.42 (m, 1 H), 7.25- 7.30 (m, 1 H), 6.79- 6.85 (m, 1 H), 6.69- 6.74 (m, 1 H), 5.24- 5.36 (m, 1 H), 4.79- 4.89 (m, 1 H), 4.64- 4.73 (m, 1 H), 4.37- 4.50 (m, 1 H), 4.16- 4.30 (m, 1 H), 3.85- 3.96 (m, 1 H), 3.69- 3.77 (m, 2 H), 3.62- 3.69 (m, 5 H), 3.53- 3.62 (m, 2 H), 2.93- 3.03 (m, 5 H), 2.74- 2.86 (m, 2 H). MS obsd. (ESI⁺) [(M + H)⁺]: 616.1. |

### Example 051: N-[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]oxy]cyclohexyl]-3-amino-propanamide

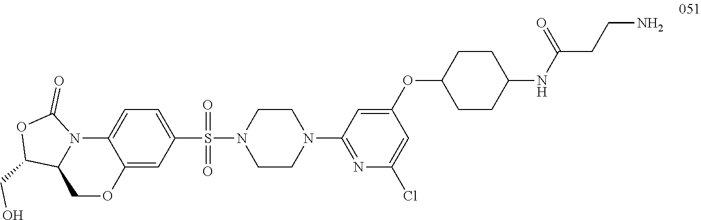 [SMILES: NCCC(=O)NC1CCC(Oc2cc(Cl)nc(N3CCN(S(=O)(=O)c4ccc5c(c4)OC[C@H]4[C@H](CO)OC(=O)N54)CC3)c2)CC1]

### Step 1. tert-Butyl N-[4-[[2-chloro-6-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-4-pyridyl]oxy]cyclohexyl]carbamate

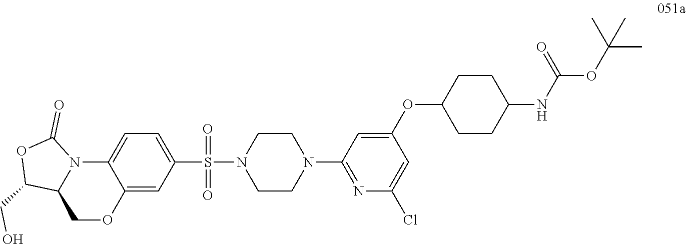 [SMILES: CC(C)(C)OC(=O)NC1CCC(Oc2cc(Cl)nc(N3CCN(S(=O)(=O)c4ccc5c(c4)OC[C@H]4[C@H](CO)OC(=O)N54)CC3)c2)CC1]

A mixture of Int-4A (93 mg, 0.252 mmol), N-[4-[(2,6-dichloro-4-pyridyl)oxy]cyclohexyl]carbamic acid tert-butyl ester (90.95 mg, 0.252 mmol) and DIPEA (162.69 mg, 219.85 μL, 1.26 mmol) in N-ethyl-2-pyrrolidinone (1 mL) was stirred at 150° C. for 3 days. After the reaction was completed, the mixture was diluted with EtOAc (20 mL) and washed with water. The organic phase was dried with anhydrous sodium sulfate and filtered. The filtrate was concentrated in vacuo to give crude compound 047a (174 mg, 100%), which was used directly in next step. MS obsd. (ESI⁺) [(M+H)⁺]: 694.2.

### Step 2. (3R,3aS)-7-[4-[4-(4-aminocyclohexoxy)-6-chloro-2-pyridyl]piperazin-1-yl]sulfonyl-3-(hydroxymethyl)-3a,4-dihydro-3H-oxazolo[4,3-c][1,4]benzoxazin-1-one

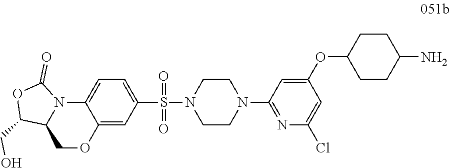 [SMILES: NC1CCC(Oc2cc(Cl)nc(N3CCN(S(=O)(=O)c4ccc5c(c4)OC[C@H]4[C@H](CO)OC(=O)N54)CC3)c2)CC1]

A mixture of compound 051a (174 mg, 0.251 mmol) and TFA (571.58 mg, 5.01 mmol) in dichloromethane (1.5 mL) was stirred at room temperature for 2 h. After the reaction was completed, the mixture was concentrated in vacuo to give crude compound 051b (148 mg, 99%), which was used directly in next step. MS obsd. (ESI⁺) [(M+H)⁺]: 594.2.

### Step 3. tert-Butyl N-[3-[[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]oxy]cyclohexyl]amino]-3-oxo-propyl]carbamate

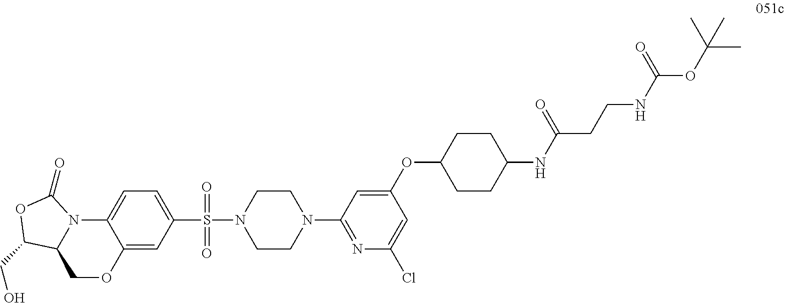 [SMILES: CC(C)(C)OC(=O)NCCC(=O)NC1CCC(Oc2cc(Cl)nc(N3CCN(S(=O)(=O)c4ccc5c(c4)OC[C@H]4[C@H](CO)OC(=O)N54)CC3)c2)CC1]

A mixture of compound mmol), 3-(tert-051b (148 mg, 0.249 butoxycarbonylamino) propionic acid (70.71 mg, 0.37 mmol), HATU (142.09 mg, 0.374 mmol) and DIPEA (96.59 mg, 0.75 mmol) in N,N-dimethylformamide (1.5 mL) at 25° C. for 16 h. After the reaction was completed, the mixture was diluted with EtOAc (20 mL) and washed with sat. NH₄Cl aqueous (20 mL). The organic layer was dried with anhydrous Na₂SO₄ and filtered. The filtrate was concentrated in vacuo to give crude compound 051c (190 mg, 100%). MS obsd. (ESI⁺) [(M+H)⁺]: 765.4.

### Step 4. N-[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]oxy]cyclohexyl]-3-amino-propanamide

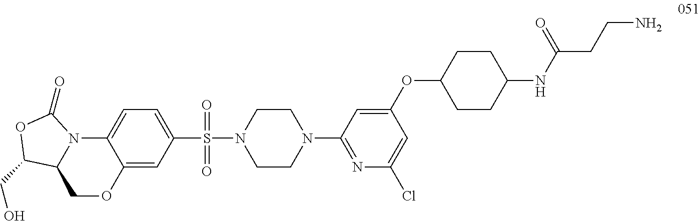 [SMILES: NCCC(=O)NC1CCC(Oc2cc(Cl)nc(N3CCN(S(=O)(=O)c4ccc5c(c4)OC[C@H]4[C@H](CO)OC(=O)N54)CC3)c2)CC1]

A mixture of compound 051c (190 mg, 0.248 mmol) and TFA (740 mg, 6.49 mmol) in dichloromethane (1.5 mL) was stirred at room temperature for 1 h. After the reaction was completed, the mixture was concentrated in vacuo to give a residue, which was purified by prep-HPLC (HCl as additive) to give Example 051 (24 mg, 14.0% yield). MS obsd. (ESI⁺) [(M+H)⁺]: 665.1.

Example 051: ¹H NMR (500 MHz, DMSO-d₆) δ ppm 8.13 (d, J=8.5 Hz, 1H), 8.04 (d, J=7.5 Hz, 1H), 7.66 (br s, 2H), 7.38 (dd, J=2.0, 8.5 Hz, 1H), 7.28 (d, J=2.0 Hz, 1H), 6.41 (s, 1H), 6.22 (s, 1H), 5.32 (t, J=5.6 Hz, 1H), 4.60 (m, 1H), 4.47 (m, 2H), 4.11 (m, 2H), 3.72 (m, 2H), 3.56 (br s, 4H), 2.96 (m, 6H), 2.43 (m, 2H), 1.99 (br d, J=9.6 Hz, 2H), 1.81 (br d, J=10.1 Hz, 2H), 1.45-1.28 (m, 4H).

### Example 052: (3R,3aR)-7-[4-[4-[[1-[2-[bis(2-aminoethyl)amino]acetyl]-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one

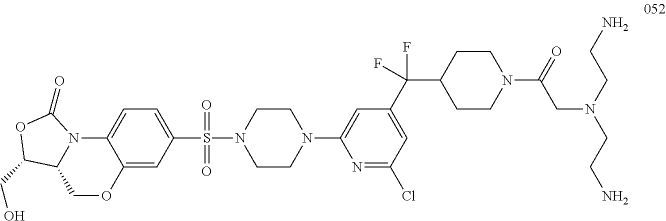 [SMILES: NCCN(CCN)CC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

### Step 1. tert-Butyl N-[2-[[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]-2-oxo-ethyl]-[2-(tert-butoxycarbonylamino)ethyl]amino]ethyl]carbamate

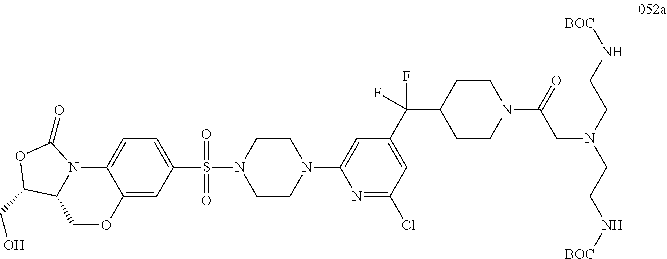 [SMILES: CC(C)(C)OC(=O)NCCN(CCNC(=O)OC(C)(C)C)CC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of Int-36 (100.06 mg, 0.277 mmol), Example 028 (85 mg, 0.138 mmol), HATU (94.74 mg, 0.249 mmol) and DIPEA (53.7 mg, 0.415 mmol in N, N-dimethylformamide (2 mL) was stirred at room temperature overnight. After the reaction was completed, the mixture was diluted with EtOAc (20 mL) and washed with water (10 mL×2). The organic phase was dried with anhydrous sodium sulfate and filtered. The filtrate was concentrated in vacuo to give crude compound 052a (130 mg, 98% yield), which was used directly in next step. MS obsd. (ESI⁺) [(M+H)⁺]: 957.6.

### Step 2. (3R,3aR)-7-[4-[4-[[1-[2-[bis(2-aminoethyl)amino]acetyl]-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one

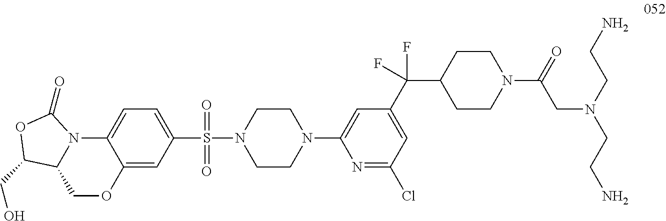 [SMILES: NCCN(CCN)CC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of compound 052a (130 mg, 0.136 mmol) and TFA (232.21 mg, 2.04 mmol) in dichloromethane (1.5 mL) was stirred at room temperature for 1 h. After the reaction was completed, the mixture was concentrated in vacuo to give residue, which was purified by prep-HPLC (HCl as additive) to give Example 052 (13 mg, 11.0% yield). MS obsd. (ESI⁺) [(M+H)⁺]: 757.4.

Example 052: ¹H NMR (400 MHz, DMSO-d₆) δ ppm 8.23 (d, J=8.7 Hz, 1H), 7.39 (dd, J=2.1, 8.7 Hz, 1H), 7.29 (d, J=2.1 Hz, 1H), 6.78 (s, 1H), 6.70 (m, 1H), 4.84 (m, 1H), 4.69 (br dd, J=3.1, 10.5 Hz, 1H), 4.44 (m, 2H), 4.23 (br t, J=10.3 Hz, 1H), 3.79 (br d, J=12.0 Hz, 1H), 3.68 (m, 6H), 3.59 (m, 2H), 2.98 (m, 4H), 2.88 (m, 1H), 2.74 (m, 8H), 2.56 (m, 1H), 2.46 (m, 1H), 1.61 (m, 2H), 1.32 (m, 1H), 1.14 (m, 1H). ¹⁹F NMR (376 MHz, CD₃OD) δ ppm −104.46 (m, 2F).

The following Examples 053 to 054 were prepared in analogy to the procedure described for the preparation of Example 052, replacing Int-36 with “RCOOH” by the reagent indicated in Table 7 in step 1.

TABLE 7
Compounds synthesis and characterization

| Example | Compounds Name and | | |
|---|---|---|---|
| No. | Structure | RCOOH | NMR and (ESI⁺) |
| 053 | (3R,3aR)-7-[4-[4-[1-(2- aminoacetyl)-4-piperidyl]- difluoro-methyl]-6-chloro-2- pyridyl]piperazin-1- yl]sulfonyl-3- (hydroxymethyl)-3a,4- dihydro-3H-oxazolo[4,3- c][1,4]benzoxazin-1-one 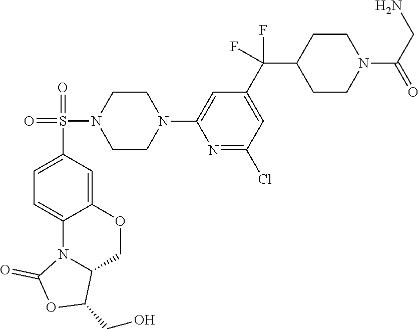 [SMILES: NCC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1] | 2-(tert- butoxycarbonyl amino)acetic acid | ¹H NMR (400 MHz, DMSO-d6) δ ppm δ ppm 8.23 (d, J = 8.7 Hz, 1H), 7.39 (dd, J = 2.1, 8.6 Hz, 1H), 7.29 (d, J = 2.1 Hz, 1H), 6.78 (s, 1H), 6.70 (s, 1H), 5.31 (br s, 1H), 4.84 (m, 1H), 4.69 (dd, J = 3.2, 10.5 Hz, 1H), 4.44 (m, 2H), 4.23 (br t, J = 10.3 Hz, 1H), 3.91 (br dd, J = 5.4, 16.4 Hz, 1H), 3.81 (m, 1H), 3.67 (m, 6H), 3.60 (br d, J = 3.7 Hz, 1H), 3.17 (s, 2H), 2.99 (m, 4H), 2.58 (m, 1H), 1.64 (br t, J = 8.9 Hz, 2H), 1.39-1.23 (m, 1H), 1.20-1.09 (m, 1H). ¹⁹F NMR (376 MHz, CD₃OD) δ ppm −109.15 (d, 1F), −109.46 (d, 1F). MS obsd. (ESI⁺) [(M + H)⁺]: 671.3. |
| 054 | (3R,3aR)-7-[4-[4-[1-(3- aminopropanoyl)-4- piperidyl]-difluoro-methyl]- 6-chloro-2-pyridyl]piperazin- 1-yl]sulfonyl-3- (hydroxymethyl)-3a,4- dihydro-3H-oxazolo[4,3- c][1,4]benzoxazin-1-one 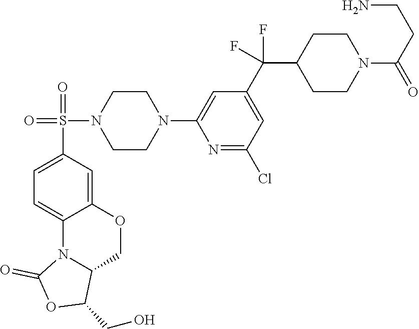 [SMILES: NCCC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1] | 3-(tert- butoxycarbonyl amino)propanoic acid | ¹H NMR (400 MHz, DMSO-d6) δ ppm δ ppm 8.23 (d, J = 8.6 Hz, 1H), 7.39 (dd, J = 2.1, 8.6 Hz, 1H), 7.29 (d, J = 2.1 Hz, 1H), 6.78 (s, 1H), 6.71 (s, 1H), 4.84 (m, 1H), 4.69 (br dd, J = 3.1, 10.6 Hz, 1H), 4.45 (m, 2H), 4.23 (m, 1H), 3.84 (br d, J = 12.8 Hz, 1H), 3.68 (m, 6H), 3.58 (m, 2H), 2.99 (m, 4H), 2.91 (m, 2H), 2.59 (m, 3H), 1.62 (br d, J = 12.1 Hz, 2H), 1.33- 1.09 (m, 2H). ¹⁹F NMR (376 MHz, CD₃OD) δ ppm −109.38 (d, 2F). MS obsd. (ESI⁺) [(M + H)⁺]: 685.4. |

### Example 055: (3R,3aR)-7-[4-[4-[[1-(2-aminoethyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one

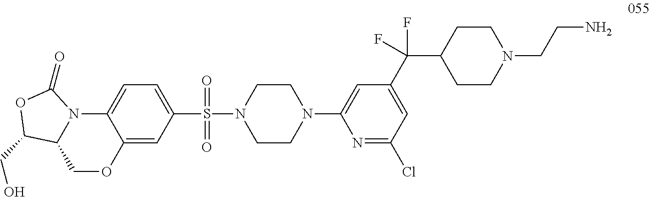 [SMILES: NCCN1CCC(C(F)(F)c2cc(Cl)nc(N3CCN(S(=O)(=O)c4ccc5c(c4)OC[C@@H]4[C@H](CO)OC(=O)N54)CC3)c2)CC1]

### Step 1. tert-Butyl N-[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]ethyl]carbamate

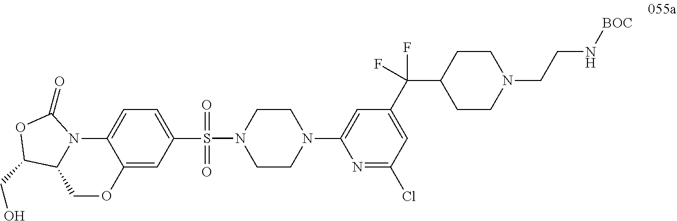 [SMILES: CC(C)(C)OC(=O)NCCN1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of Example 028 (60 mg, 0.098 mmol), N-(2-bromoethyl) carbamic acid tert-butyl ester (26.28 mg, 0.117 mmol) and DIPEA (37.88 mg, 0.293 mmol) in acetonitrile (1.5 mL) was stirred at rt for 16 h. The mixture was diluted with EtOAc (20 mL) and washed with water. The organic layer was dried over anhydrous sodium sulfate and filtered. The filtrate was concentrated in vacuo to give crude product compound 055a (72 mg, 97.0% yield), which was used directly in next step. MS obsd. (ESI⁺) [(M+H)⁺]: 757.4.

### Step 2. (3R,3aR)-7-[4-[4-[[1-(2-aminoethyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one

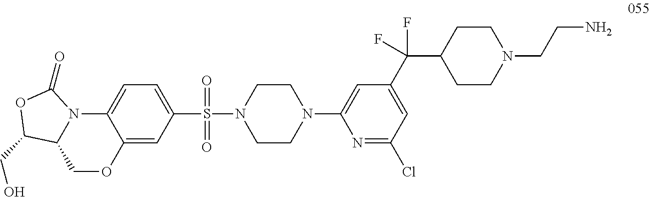 [SMILES: NCCN1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of compound 055a (72 mg, 0.095 mmol) and TFA (216.83 mg, 1.9 mmol) in dichloromethane (2 mL) was stirred at rt for 1 h. The mixture was concentrated to give a residue which was purified by prep-HPLC (HCl as additive) to give Example 055 (7.6 mg, 12% yield). MS obsd. (ESI⁺) [(M+H)⁺]: 657.4.

Example 055: ¹H NMR (400 MHz, DMSO-d₆) δ ppm 8.23 (d, J=8.6 Hz, 1H), 7.39 (dd, J=2.1, 8.7 Hz, 1H), 7.29 (d, J=2.1 Hz, 1H), 6.78 (s, 1H), 6.70 (s, 1H), 5.33 (m, 1H), 4.84 (m, 1H), 4.68 (dd, J=3.1, 10.5 Hz, 1H), 4.44 (dt, J=3.1, 9.4 Hz, 1H), 4.23 (t, J=10.3 Hz, 1H), 3.68 (m, 6H), 3.59 (m, 1H), 2.98 (m, 4H), 2.85 (m, 4H), 2.41 (m, 2H), 2.17 (m, 1H), 2.00 (m, 1H), 1.90 (m, 1H), 1.52 (m, 1H), 1.40 (m, 1H). ¹⁹F NMR (376 MHz, METHANOL-d₄) δ ppm −109.01 (m, 2F).

### Example 056: 2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]acetamide

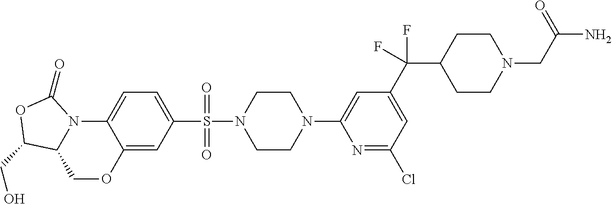 [SMILES: NC(=O)CN1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of Example 028 (60 mg, 0.098 mmol), 2-bromoacetamide (17.52 mg, 0.127 mmol) and DIPEA (63.14 mg, 0.489 mmol) in acetonitrile (1.5 mL) was stirred at rt for 16 h. After the reaction was completed, the mixture was concentrated in vacuo to give a residue which was purified by prep-HPLC (HCl as additive) to give Example 056 (18.7 mg, 26% yield). MS obsd. (ESI⁺) [(M+H)⁺]: 671.1.

Example 056: ¹H NMR (400 MHz, DMSO-d₆) δ ppm 8.23 (d, J=8.6 Hz, 1H), 7.91 (br s, 1H), 7.68 (br s, 1H), 7.39 (dd, J=2.1, 8.6 Hz, 1H), 7.29 (d, J=2.0 Hz, 1H), 6.79 (s, 1H), 6.72 (s, 1H), 5.31 (br s, 1H), 4.84 (m, 1H), 4.69 (dd, J=3.2, 10.5 Hz, 1H), 4.44 (m, 1H), 4.22 (t, J=10.3 Hz, 1H), 3.83 (br s, 2H), 3.68 (m, 6H), 3.58 (br dd, J=3.5, 12.3 Hz, 1H), 3.47 (m, 2H), 2.99 (br s, 6H), 1.87-1.63 (m, 4H). ¹⁹F NMR (376 MHz, CD₃OD) δ ppm −108.94 (d, 2F).

### Example 057: N-[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]-2-oxo-ethyl]-2-amino-acetamide

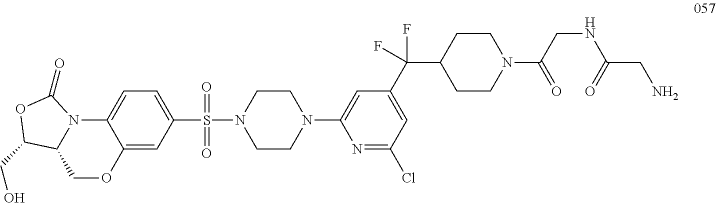 [SMILES: NCC(=O)NCC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

### Step 1. tert-Butyl N-[2-[[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]-2-oxo-ethyl]amino]-2-oxo-ethyl]carbamate

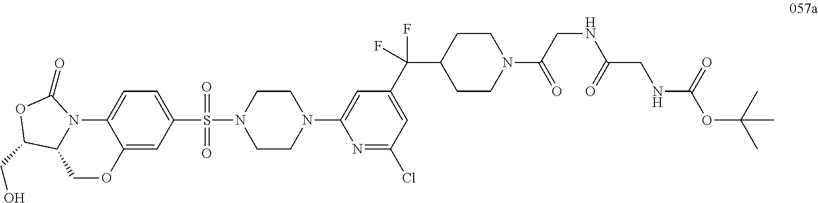 [SMILES: CC(C)(C)OC(=O)NCC(=O)NCC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of Example 053 (87 mg, 0.130 mmol), Boc-glycine (34.06 mg, 0.194 mmol), DIPEA (50.26 mg, 0.389 mmol) and 2-(7-aza-1 h-benzotriazole-1-yl)-1,1,3,3-tetramethyluronium hexafluorophosphate (88.72 mg, 0.23 mmol) in N,N-dimethylformamide (2 mL) was stirred at rt for 16 h. After the reaction was completed, the mixture was diluted with EtOAc (20 mL) and washed with water (10 mL×2). The organic phase was dried with anhydrous sodium sulfate and filtered. The filtrate was concentrated in vacuo to give compound 057a (100 mg, 93.0% yield), which was used directly in next step. MS obsd. (ESI⁺) [(M+H−Boc)⁺]: 728.3.

### Step 2. N-[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]-2-oxo-ethyl]-2-amino-acetamide

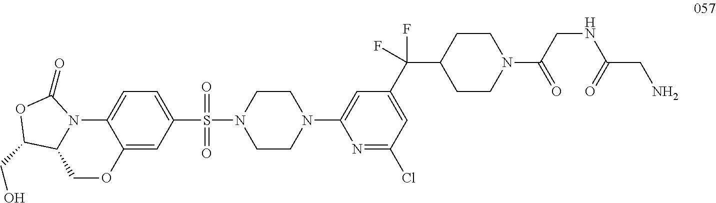 [SMILES: NCC(=O)NCC(=O)N1CCC(C(F)(F)c2cc(Cl)nc(N3CCN(S(=O)(=O)c4ccc5c(c4)OC[C@@H]4[C@H](CO)OC(=O)N54)CC3)c2)CC1]

A mixture of compound 057a (100 mg, 0.121 mmol) and TFA (206.49 mg, 1.81 mmol) in dichloromethane (2 mL) was stirred at room temperature for 1 h. After the reaction was completed, the mixture was concentrated in vacuo to give crude, which was purified by prep-HPLC (HCl as additive) to give Example 057 (18 mg, 18%). MS obsd. (ESI⁺) [(M+H)⁺]: 727.9.

Example 057: ¹H NMR (400 MHz, DMSO-d₆) δ ppm 8.16 (d, J=8.7 Hz, 1H), 7.32 (dd, J=2.0, 8.6 Hz, 1H), 7.22 (d, J=1.8 Hz, 1H), 6.71 (s, 1H), 6.64 (s, 1H), 4.77 (m, 1H), 4.62 (br dd, J=2.9, 10.5 Hz, 1H), 4.37 (m, 2H), 4.16 (m, 1H), 3.91 (m, 1H), 3.75 (br d, J=16.3 Hz, 1H), 3.61 (m, 7H), 3.51 (m, 4H), 2.89 (m, 5H), 1.54 (m, 2H), 1.30-1.01 (m, 2H). ¹⁹F NMR (376 MHz, CD₃OD) δ ppm −104.30 (m, 2F).

### Example 058 and 059: (1S,2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride (Example 058) and (1R,2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride (Example 059)

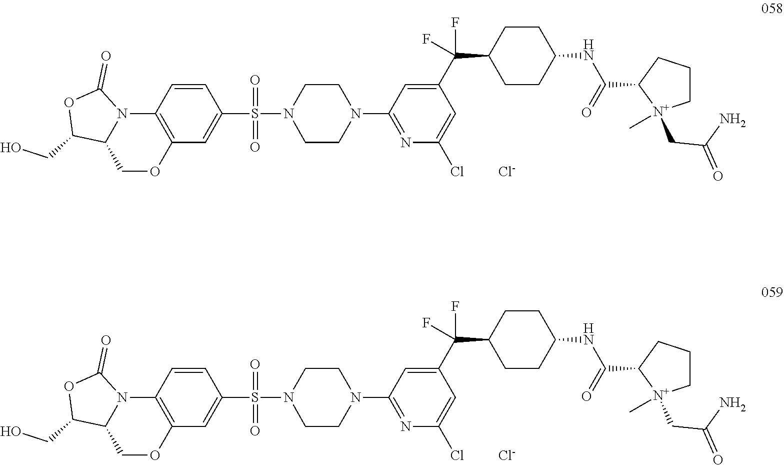 [SMILES: C[N@@+]1(CC(N)=O)CCC[C@H]1C(=O)N[C@H]1CC[C@H](C(F)(F)c2cc(Cl)nc(N3CCN(S(=O)(=O)c4ccc5c(c4)OC[C@@H]4[C@H](CO)OC(=O)N54)CC3)c2)CC1 ; [Cl-] ; C[N@+]1(CC(N)=O)CCC[C@H]1C(=O)N[C@H]1CC[C@H](C(F)(F)c2cc(Cl)nc(N3CCN(S(=O)(=O)c4ccc5c(c4)OC[C@@H]4[C@H](CO)OC(=O)N54)CC3)c2)CC1 ; [Cl-]]

To a solution of Int-2B (120.0 mg, 0.260 mmol) and Int-37 (143.19 mg, 0.390 mmol) in DMSO (0.500 mL) was added DIPEA (0.23 mL, 1.29 mmol), the mixture was stirred at 110° C. for 12 h. After the reaction was completed, the reaction was purified by Pre-HPLC (HCl as additive) to give Example 058 (22.5 mg, 10.3% yield, faster eluted) as a white solid and Example 059 (12.6 mg, 5.72% yield, slower eluted) as a white solid.

Example 058: ¹H NMR (400 MHz, DMSO-d₆) δ ppm 8.64-8.98 (m, 1H) 8.49 (s, 1H) 8.23 (d, J=8.68 Hz, 1H) 7.92-8.10 (m, 1H) 7.62 (br s, 1H) 7.39 (dd, J=8.56, 2.08 Hz, 1H) 7.29 (d, J=1.83 Hz, 1H) 6.79 (s, 1H) 6.71 (s, 1H) 4.79-4.90 (m, 1H) 4.68 (br dd, J=10.51, 3.06 Hz, 1H) 4.55 (br d, J=2.32 Hz, 1H) 4.40-4.47 (m, 1H) 4.15-4.28 (m, 3H) 3.80-3.93 (m, 1H) 3.54-3.77 (m, 8H) 3.24 (br s, 3H) 2.97 (br s, 4H) 2.04-2.24 (m, 4H) 1.57-1.93 (m, 5H) 1.33-1.55 (m, 1H) 1.04-1.29 (m, 4H). ¹⁹F NMR (376 MHz, DMSO-d₆) δ ppm −104.24-−103.22 (m, 2 F); MS obsd. (ESI⁺) [(M−Cl)⁺]: 796.2.

Example 059: ¹H NMR (400 MHz, DMSO-d₆) δ ppm 8.50 (s, 1H) 8.23 (d, J=8.68 Hz, 1H) 7.71 (br s, 1H) 7.39 (dd, J=8.68, 2.08 Hz, 1H) 7.29 (d, J=2.08 Hz, 1H) 6.79 (s, 1H) 6.71 (s, 1H) 4.84 (dt, J=8.41, 4.17 Hz, 1H) 4.68 (br dd, J=10.82, 3.12 Hz, 1H) 4.35-4.54 (m, 2H) 4.07-4.33 (m, 3H) 3.82-4.00 (m, 1H) 3.52-3.81 (m, 8H) 3.16 (s, 3H) 2.97 (br s, 4H) 2.01-2.29 (m, 5H) 1.59-1.96 (m, 5H) 1.14-1.29 (m, 4H). ¹⁹F NMR (376 MHz, DMSO-d₆) δ ppm −104.24-−103.06 (m, 2 F). MS obsd. (ESI⁺) [(M−Cl)⁺]: 796.2.

### Example 060 and 061: (1S,2S)—N-[4-[[2-[4-[[(3S,3aR)-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide; chloride (Example 060) and (1R,2S)—N-[4-[[2-[4-[[(3S,3aR)-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride (Example 061)

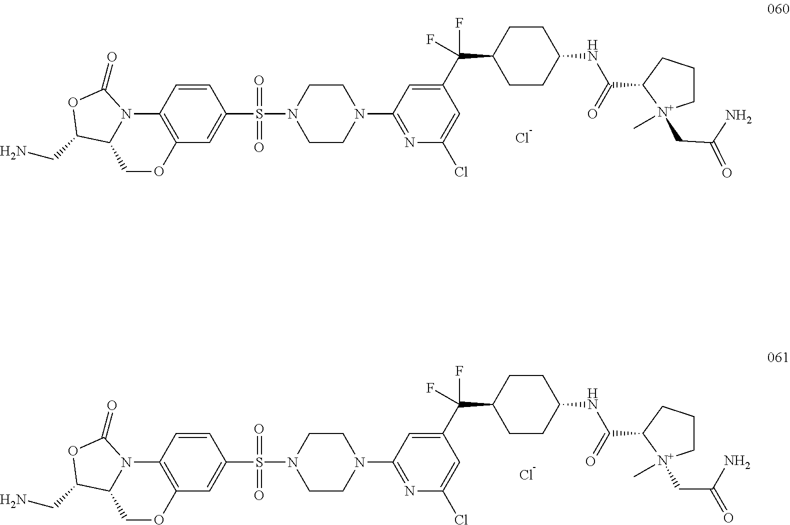 [SMILES: C[N@@+]1(CC(N)=O)CCC[C@H]1C(=O)N[C@H]1CC[C@H](C(F)(F)c2cc(Cl)nc(N3CCN(S(=O)(=O)c4ccc5c(c4)OC[C@@H]4[C@H](CN)OC(=O)N54)CC3)c2)CC1 ; Cl ; C[N@+]1(CC(N)=O)CCC[C@H]1C(=O)N[C@H]1CC[C@H](C(F)(F)c2cc(Cl)nc(N3CCN(S(=O)(=O)c4ccc5c(c4)OC[C@@H]4[C@H](CN)OC(=O)N54)CC3)c2)CC1 ; Cl]

The title compound was prepared in analogy to the preparation of Example 058 & 059 by using Int-9 instead of Int-2B followed de-Cbz protection using TFA as solvent at 60° C. for 4 h. The final residue was purified by prep-HPLC (HCl as additive) to afford Example 060 (16.5 mg, 21.72% yield, faster eluted) as a white powder and Example 061 (17.6 mg, 23.5% yield, slower eluted) as a white solid.

Example 060: ¹H NMR (400 MHz, DMSO-d₆) δ ppm 8.67 (td, J=8.83, 2.38 Hz, 1H) 8.40-8.57 (m, 3H) 8.20 (d, J=8.68 Hz, 1H) 7.98 (br s, 1H) 7.62 (s, 1H) 7.42 (dd, J=8.62, 1.90 Hz, 1H) 7.32 (d, J=1.96 Hz, 1H) 6.80 (s, 1H) 6.72 (s, 1H) 5.16 (br t, J=10.03 Hz, 1H) 4.69 (br d, J=7.82 Hz, 1H) 4.45-4.61 (m, 2H) 4.08-4.34 (m, 3H) 3.81-3.93 (m, 1H) 3.59-3.77 (m, 5H) 3.42-3.50 (m, 1H) 3.13-3.29 (m, 5H) 2.97 (br s, 4H) 2.11-2.27 (m, 3H) 2.00-2.09 (m, 1H) 1.60-1.90 (m, 4H) 1.10-1.54 (m, 5H). ¹⁹F NMR (376 MHz, DMSO-d₆) δ ppm −104.69-−102.96 (m, 2 F). MS obsd. (ESI⁺) [(M−Cl)⁺]: 795.3.

Example 061: ¹H NMR (400 MHz, DMSO-d₆) δ ppm 8.83 (br d, J=7.46 Hz, 1H) 8.56 (br s, 3H) 8.13-8.27 (m, 2H) 7.74 (br s, 1H) 7.41 (dd, J=8.56, 1.96 Hz, 1H) 7.31 (d, J=1.96 Hz, 1H) 6.81 (s, 1H) 6.72 (s, 1H) 5.11-5.25 (m, 1H) 4.69 (br dd, J=10.03, 2.69 Hz, 1H) 4.47-4.57 (m, 2H) 4.13-4.27 (m, 4H) 3.87-3.98 (m, 1H) 3.77 (br dd, J=7.34, 4.28 Hz, 1H) 3.66 (br s, 4H) 3.41-3.55 (m, 1H) 3.18 (s, 5H) 2.88-3.03 (m, 4H) 2.03-2.24 (m, 4H) 1.77-1.94 (m, 2H) 1.58-1.72 (m, 2H) 1.09-1.31 (m, 4H). ¹⁹F NMR (376 MHz, DMSO-d₆) δ ppm −104.70-−102.84 (m, 2 F). MS obsd. (ESI⁺) [(M−Cl)⁺]: 795.3.

### Example 062: (2S)-1-(2-aminoethyl)-N-[4-[[2-chloro-6-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-4-pyridyl]-difluoro-methyl]cyclohexyl]-1-methyl-pyrrolidin-1-ium-2-carboxamide;chloride

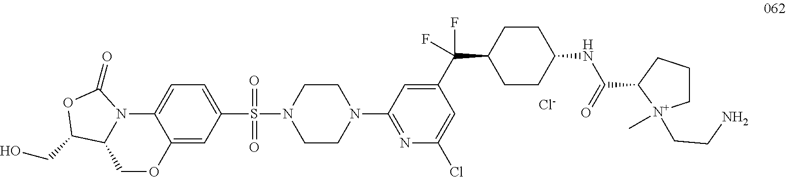 [SMILES: C[N+]1(CCN)CCC[C@H]1C(=O)N[C@H]1CC[C@H](C(F)(F)c2cc(Cl)nc(N3CCN(S(=O)(=O)c4ccc5c(c4)OC[C@@H]4[C@H](CO)OC(=O)N54)CC3)c2)CC1 ; [Cl-]]

### Step 1: Benzyl N-[2-[(2S)-2-[[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]carbamoyl]pyrrolidin-1-yl]ethyl]carbamate

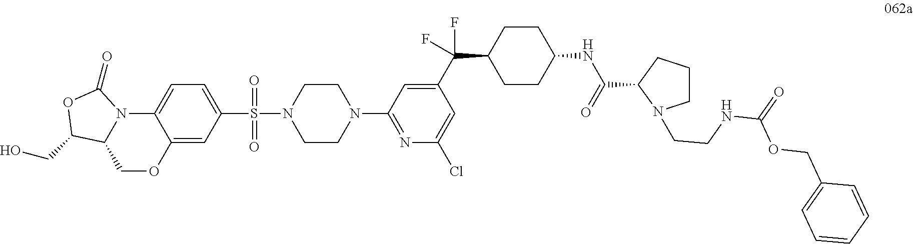 [SMILES: O=C(NCCN1CCC[C@H]1C(=O)N[C@H]1CC[C@H](C(F)(F)c2cc(Cl)nc(N3CCN(S(=O)(=O)c4ccc5c(c4)OC[C@@H]4[C@H](CO)OC(=O)N54)CC3)c2)CC1)OCc1ccccc1]

To a solution of Int-38 (100.0 mg, 0.180 mmol) and Int-2B (64.87 mg, 0.180 mmol) in DMSO (0.500 mL) was added DIPEA (0.2 mL). The mixture was stirred at 110° C. for 12 h. After the reaction was completed, the mixture was purified by prep-HPLC (TFA as additive) to afford Example 062a (50 mg, 31.6% yield) as white solid. MS obsd. (ESI⁺) [(M+H)⁺]: 902.3.

### Step 2: Benzyl N-[2-[(2S)-2-[[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]carbamoyl]-1-methyl-pyrrolidin-1-ium-1-yl]ethyl]carbamate; chloride

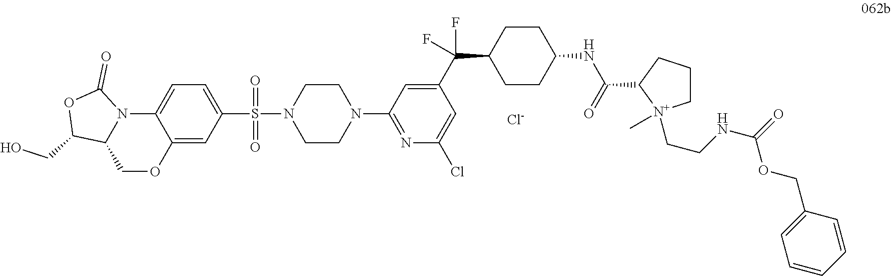

A mixture of compound 062a (50.0 mg, 0.060 mmol), NaHCO₃ (13.96 mg, 0.170 mmol) and MeI (0.2 mL) in MeCN (2 mL) was stirred at 40° C. for 2 h. After the reaction was completed, the mixture was filtered and concentrated under reduced pressure to give crude compound 062b (50 mg, 98.2% yield) as yellow oil. MS obsd. (ESI⁺) [(M−Cl)⁺]: 916.4.

### Step 3: (2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-aminoethyl)-1-methyl-pyrrolidin-1-ium-2-carboxamide;chloride

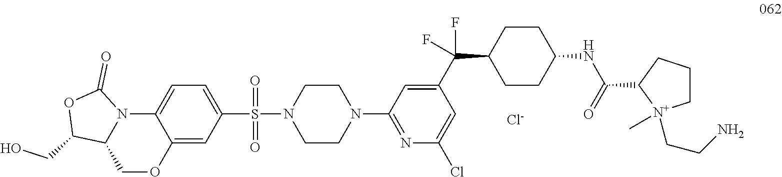

A solution of compound 062b (50.0 mg, 0.050 mmol) in TFA (1.0 mL) was stirred at 50° C. for 2 h. After the reaction was completed, the mixture was concentrated in vacuo to give a residue, which was purified by prep-HPLC (HCl as additive) to give Example 062 (18.5 mg, 39.4% yield) as yellow solid. MS obsd. (ESI⁺) [(M−Cl)⁺]: 782.3.

Example 062: ¹H NMR (400 MHz, DMSO-d₆) δ ppm 1.17-1.32 (m, 4H) 1.69 (br d, J=8.80 Hz, 2H) 1.83 (br s, 1H) 1.91 (br s, 1H) 2.10-2.24 (m, 4H) 2.45 (br d, J=5.62 Hz, 1H) 2.98 (br s, 4H) 3.06 (s, 2H) 3.15 (s, 1H) 3.30-3.34 (m, 1H) 3.51-3.89 (m, 13H) 4.20-4.29 (m, 1H) 4.39-4.57 (m, 2H) 4.69 (dd, J=10.45, 2.87 Hz, 1H) 4.80-4.91 (m, 1H) 6.72 (s, 1H) 6.80 (s, 1H) 7.29 (d, J=1.96 Hz, 1H) 7.39 (dd, J=8.62, 2.02 Hz, 1H) 8.23 (d, J=8.68 Hz, 1H) 8.41-8.65 (m, 3H) 8.96-9.15 (m, 1H). ¹⁹F NMR (376 MHz, DMSO-d₆) δ ppm −104.43-−102.68 (m, 1 F).

### Example 063:4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-amino-2-oxo-ethyl)pyrrolidin-1-ium-1-yl]ethyl]cyclohexanecarboxamide; chloride

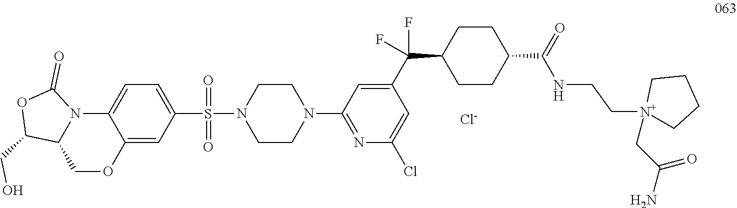 [SMILES: NC(=O)C[N+]1(CCNC(=O)[C@H]2CC[C@H](C(F)(F)c3cc(Cl)nc(N4CCN(S(=O)(=O)c5ccc6c(c5)OC[C@@H]5[C@H](CO)OC(=O)N65)CC4)c3)CC2)CCCC1 ; [Cl-]]

### Step 1: N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-3-pyrrolidin-1-yl-propanamide

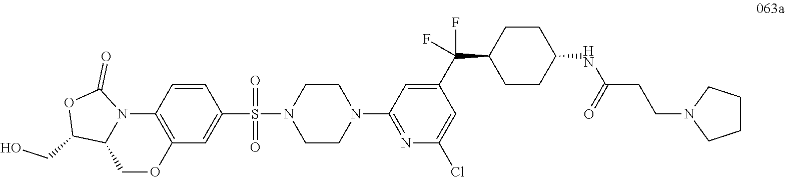 [SMILES: O=C(CCN1CCCC1)N[C@H]1CC[C@H](C(F)(F)c2cc(Cl)nc(N3CCN(S(=O)(=O)c4ccc5c(c4)OC[C@@H]4[C@H](CO)OC(=O)N54)CC3)c2)CC1]

A mixture of DIPEA (138.37 mg, 1.07 mmol), Int-2B (131.82 mg, 0.357 mmol) and Int-39 (150 mg, 0.357 mmol) in DMSO (2 mL) at 100° C. for 16 h. After the reaction was completed, the mixture was diluted with EtOAc (20 mL), washed with water (20 mL) and brine (20 mL). The organic phase was dried over anhydrous sodium sulfate and filtered. The filtrate was concentrated in vacuo to give crude compound 063a (268 mg, 99% yield), which was used in next step directly. MS obsd. (ESI⁺) [(M+H)⁺]: 753.2.

### Step 2. 4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-amino-2-oxo-ethyl)pyrrolidin-1-ium-1-yl]ethyl]cyclohexanecarboxamide;chloride

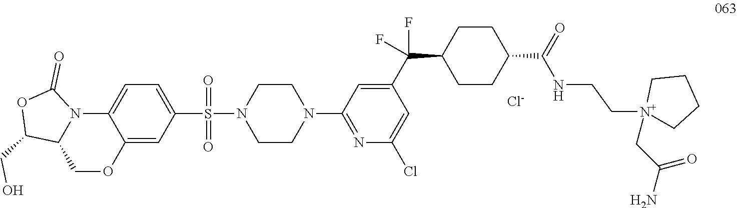 [SMILES: NC(=O)C[N+]1(CCNC(=O)[C@H]2CC[C@H](C(F)(F)c3cc(Cl)nc(N4CCN(S(=O)(=O)c5ccc6c(c5)OC[C@@H]5[C@H](CO)OC(=O)N65)CC4)c3)CC2)CCCC1 ; [Cl-]]

A mixture of compound 063a (260 mg, 0.345 mmol), 2-bromoacetamide (61.9 mg, 0.449 mmol) and DIPEA (133.83 mg, 1.04 mmol) in acetonitrile (2.5 mL) was stirred at room temperature for 18 h. After the reaction was completed, the mixture was concentrated in vacuo to give a residue, which was purified by prep-HPLC (HCl as additive) to give Example 063 (70 mg, 24% yield). MS obsd. (ESI⁺) [(M−Cl)⁺]: 810.2.

Example 063: ¹H NMR (500 MHz, DMSO-d₆) δ ppm 8.55 (m, 2H), 8.38 (m, 1H), 8.13 (d, J=8.5 Hz, 1H), 7.62 (s, 1H), 7.39 (dd, J=2.1, 8.6 Hz, 1H), 7.29 (d, J=2.0 Hz, 1H), 6.79 (s, 1H), 6.70 (s, 1H), 4.61 (m, 1H), 4.48 (m, 1H), 4.21 (br s, 2H), 4.11 (m, 2H), 3.75 (m, 1H), 3.67 (m, 8H), 3.58 (br t, J=5.6 Hz, 2H), 3.44 (m, 2H), 2.97 (br t, J=4.4 Hz, 4H), 2.39 (m, 1H), 2.16 (m, 1H), 2.04 (m, 5H), 1.78 (br d, J=11.0 Hz, 2H), 1.66 (br d, J=11.9 Hz, 2H), 1.36-1.22 (m, 2H), 1.20-1.06 (m, 2H).

### Example 064:4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-aminoethyl)pyrrolidin-1-ium-1-yl]ethyl]cyclohexanecarboxamide; chloride

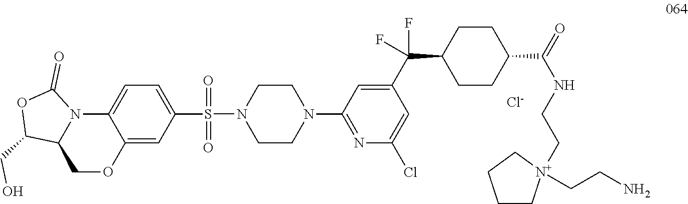 [SMILES: NCC[N+]1(CCNC(=O)[C@H]2CC[C@H](C(F)(F)c3cc(Cl)nc(N4CCN(S(=O)(=O)c5ccc6c(c5)OC[C@H]5[C@H](CO)OC(=O)N65)CC4)c3)CC2)CCCC1 ; [Cl-]]

### Step 1. N-[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-3-pyrrolidin-1-yl-propanamide

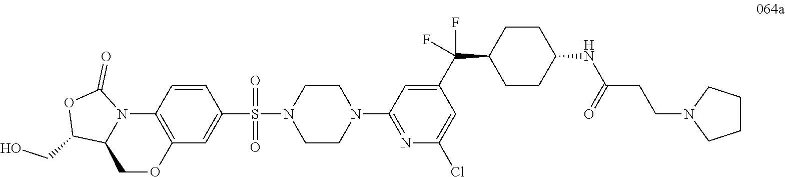 [SMILES: O=C(CCN1CCCC1)N[C@H]1CC[C@H](C(F)(F)c2cc(Cl)nc(N3CCN(S(=O)(=O)c4ccc5c(c4)OC[C@H]4[C@H](CO)OC(=O)N54)CC3)c2)CC1]

A mixture of Int-39 (120 mg, 0.286 mmol), Int-4B (105.46 mg, 0.286 mmol) and DIPEA (110.69 mg, 0.856 mmol) was stirred at 100° C. for 18 h. After the reaction was completed, the mixture was diluted with EtOAc (30 mL) and washed with NH₄Cl aqueous solution. The organic phase was dried over anhydrous sodium sulfate and filtered. The filtrate was concentrated in vacuo to give the crude compound 064a (215 mg, 100% yield), which was used in next step directly. MS (ESI⁺) obsd. [(M+H)⁺]: 753.4.

### Step 2. tert-Butyl N-[2-[1-[2-[[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexanecarbonyl]amino]ethyl]pyrrolidin-1-ium-1-yl]ethyl]carbamate;chloride

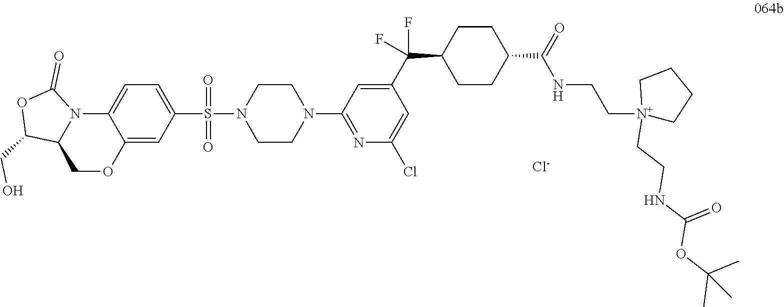 [SMILES: CC(C)(C)OC(=O)NCC[N+]1(CCNC(=O)[C@H]2CC[C@H](C(F)(F)c3cc(Cl)nc(N4CCN(S(=O)(=O)c5ccc6c(c5)OC[C@H]5[C@H](CO)OC(=O)N65)CC4)c3)CC2)CCCC1 ; [Cl-]]

A mixture of compound 064a (215 mg, 0.285 mmol), N-(2-bromoethyl) carbamic acid tert-butyl ester (383.78 mg, 1.71 mmol) and K₂CO₃ (157.79 mg, 1.14 mmol) in N,N-dimethylformamide (2 mL) was stirred at 90° C. for 16 h. After the reaction was completed, the mixture was diluted with EtOAc (20 mL) and washed with sat. NH₄Cl aqueous solution (20 mL). The organic phase was dried over anhydrous sodium sulfate and filtered. The filtrate was concentrated in vacuo to give crude compound 064b (256 mg, 100% yield), which was used in next step directly. MS (ESI⁺) obsd. [(M−Cl)⁺]: 896.3.

### Step 3. 4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-aminoethyl)pyrrolidin-1-ium-1-yl]ethyl]cyclohexanecarboxamide;chloride

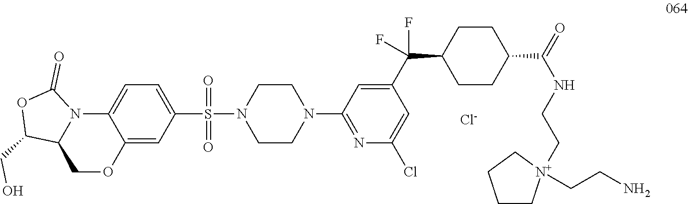 [SMILES: NCC[N+]1(CCNC(=O)[C@H]2CC[C@H](C(F)(F)c3cc(Cl)nc(N4CCN(S(=O)(=O)c5ccc6c(c5)OC[C@H]5[C@H](CO)OC(=O)N65)CC4)c3)CC2)CCCC1 ; [Cl-]]

A mixture compound 064b (256 mg, 0.285 mmol) and TFA (740 mg, 6.49 mmol) in dichloromethane (extra dry, 2 mL) was stirred at room temperature for 1 h. The mixture was concentrated in vacuo and then purified by HPLC (HCl as additive) to give Example 064 (2 mg, 1% yield). MS (ESI⁺) obsd. [(M−Cl)⁺]: 796.2.

Example 064: ¹H NMR (500 MHz, DMSO-d₆) δ ppm 8.14 (m, 1H), 8.12 (m, 1H), 7.40 (dd, J=2.0, 8.5 Hz, 1H), 7.30 (d, J=2.0 Hz, 1H), 6.79 (s, 1H), 6.71 (m, 1H), 5.34 (t, J=5.7 Hz, 1H), 4.62 (m, 1H), 4.49 (br d, J=4.6 Hz, 1H), 4.10 (m, 2H), 3.77 (m, 2H), 3.69 (br d, J=5.0 Hz, 2H), 3.66 (m, 4H), 3.58 (m, 2H), 3.18 (m, 2H), 2.98 (m, 4H), 2.18 (m, 2H), 2.08 (m, 6H), 1.76 (m, 2H), 1.68 (m, 2H), 1.32 (m, 4H), 1.13 (m, 4H). ¹⁹F NMR (376 MHz, CD₃OD) δ ppm −109.12 (d, 2F).

Compound F-1, disclosed in the literature (Bioorganic Chemistry 102 (2020) 104055) and reported having LPS synthesis pathway inhibition activity and displaying antibiotic activity against efflux-deficient E. coli strains, was chosen as reference compound in present invention.

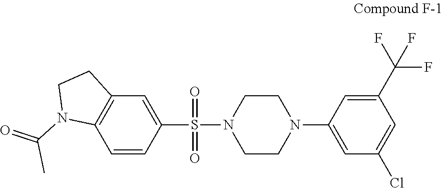 [SMILES: CC(=O)N1CCc2cc(S(=O)(=O)N3CCN(c4cc(Cl)cc(C(F)(F)F)c4)CC3)ccc21]

### Biological Examples

### Example 065: Minimal Inhibitory Concentration Protocol (MIC) Assay:

The antibacterial activity of the compounds of the present invention was evaluated against the commonly used quality control strain Escherichia coli ATCC 25922 and the rifampin-resistant mutant strain Klebsiella pneumonia ATCC 43816. Both were originally derived from human clinical samples and are available from ATCC (American Type Culture Collection). The in vitro potency of compounds to inhibit Escherichia coli (ATCC 25922) and Klebsiella pneumonia (ATCC 43816) growth was assessed by the MIC (Minimal Inhibitory Concentration) broth dilution method. Specifically compound dilutions were prepared from 10 mM DMSO stock solutions as follows: i) serial 2-fold dilution in 20 μL DMSO were prepared in a master plate (Greiner, Cat No: 651201), ii) 180 μL sterile distilled water was added to each aliquot and iii) 10 μL diluted compounds were transferred into a new assay plate (Costar, 3599).

Vials of each test microorganisms were maintained frozen in the vapor phase of a liquid nitrogen freezer. Single-use frozen vials of the two strains Escherichia coli ATCC 25922 (KWIKSTIK, 0335K) and Klebsiella pneumonia ATCC 43816, with predetermined CFU/mL, were taken out from the freezer, thawed at room temperature, and diluted in Cation-Adjusted Mueller Hinton Broth (CAMHB) to achieve a final inoculum of 5×10⁵ CFU/mL. 90 μL bacteria containing broth was dispensed to the assay plate containing the pre-dispensed compound dilutions and mixed by pipetting 5 times.

Then the assay plates were incubated for 20 hours at 35° C. in ambient air. Following incubation, the MIC (μg/mL), the lowest concentration of drug that inhibits visible growth of the microorganism was read with the help of a magnification mirror and recorded.

TABLE 8
MIC values of the compounds of this invention
against E. coli and K. pneumonia

MIC (μg/mL)

| | Escherichia | Klebsiella |
|---|---|---|
| Example | coli | pneumoniae |
| No. | ATCC25922 | ATCC43816 |
| F-1 | >24 | >24 |
| 001 | 0.46 | 0.46 |
| 002 | 0.66 | 1.32 |
| 003 | 0.97 | 1.94 |
| 004 | 0.52 | 0.26 |
| 005 | 0.97 | 1.0 |
| 006 | 0.44 | 0.88 |
| 007 | 0.46 | 0.91 |
| 008 | 0.50 | 0.50 |
| 009 | 0.66 | 0.66 |
| 010 | 0.98 | 0.98 |
| 011 | 1.40 | 2.87 |
| 012 | 0.61 | N.A. |
| 013 | 0.47 | 0.24 |
| 014 | 0.51 | 0.51 |
| 015 | 0.50 | 0.76 |
| 016 | 1.90 | 1.86 |
| 017 | 0.57 | 0.57 |
| 018 | 0.28 | 0.28 |
| 019 | 0.29 | 0.28 |
| 020 | 0.29 | 0.29 |
| 021 | 0.26 | 0.51 |
| 022 | 0.44 | 0.30 |
| 023 | 0.33 | 0.33 |
| 024 | 0.66 | 1.32 |
| 025 | 0.91 | 1.82 |
| 026 | 1.10 | 1.67 |
| 027 | 2.10 | 2.14 |
| 028 | 0.57 | 0.57 |
| 029 | 1.1 | 1.138 |
| 030 | 0.49 | 0.492 |
| 031 | 0.25 | 1.008 |
| 032 | 0.24 | 0.241 |
| 033 | 1.00 | 1.55 |
| 034 | 0.28 | 0.57 |
| 035 | 2.20 | 2.19 |
| 036 | 0.14 | 0.14 |
| 037 | 1.1 | 1.14 |
| 038 | 0.57 | 0.57 |
| 039 | 0.87 | 1.16 |
| 040 | 0.29 | 0.29 |
| 041 | 2.40 | 2.40 |
| 042 | 2.20 | 2.21 |
| 043 | 4.30 | 8.64 |
| 044 | 2.40 | 2.41 |
| 045 | 0.63 | 0.63 |
| 046 | 0.32 | 0.32 |
| 047 | 0.58 | 0.59 |
| 048 | 0.64 | 0.64 |
| 049 | 0.30 | 0.29 |
| 050 | 0.48 | 0.48 |
| 051 | 3.70 | 3.65 |
| 052 | 0.68 | 1.36 |
| 053 | 0.61 | 0.61 |
| 054 | 0.62 | 0.62 |
| 055 | 0.30 | 0.30 |
| 056 | 0.31 | 0.23 |
| 057 | 0.66 | 0.66 |
| 058 | 1.30 | 1.32 |
| 059 | 0.66 | 0.66 |
| 060 | 0.51 | 0.51 |
| 061 | 0.68 | 0.34 |
| 062 | 0.67 | 0.67 |
| 063 | 2 | 2.01 |
| 064 | 2.8 | 2.85 |

### Example 066: Human Microsome Stability Assay

The human microsomal stability assay is used for early assessment of metabolic stability of compounds of present invention in human liver microsomes. Human liver microsomes (Cat. NO.: 452117, Corning, USA) were pre-incubated with test compound for 10 minutes at 37° C. in 100 mM potassium phosphate buffer, pH 7.4. The reactions were initiated by adding NADPH regenerating system. The final incubation mixtures contained 1 μM compound, 0.5 mg/mL liver microsomal protein, 1 mM MgCl₂ and 1 mM NADPH in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 9 below.

$$CLh ( mL / min / kg ) = \frac{\begin{matrix}Clint * protein / liver ( mg / g ) * \\ liver / body weight ( g / kg ) / 1000 * liver blood flow\end{matrix}}{\begin{matrix}Liver blood flow + Clint * \\ protein / liver ( mg / g ) * liver / body weight ( g / kg ) / 1000\end{matrix}}$$

TABLE 9
Human microsome clearance values of
the compounds of this invention

| Example | Clearance of Human |
|---|---|
| No. | microsome (mL/min/kg) |
| F-1 | 14.0 |
| 002 | 10.0 |
| 006 | 12.5 |
| 007 | 9.6 |
| 009 | 6.2 |
| 025 | 12.2 |
| 026 | 10.8 |
| 028 | 10.5 |
| 029 | 11.6 |
| 033 | 13.6 |
| 034 | 12.7 |
| 035 | 10.9 |
| 037 | 10.9 |
| 038 | 11.1 |
| 039 | 6.2 |
| 042 | 6.7 |
| 043 | 6.2 |
| 044 | 7.0 |
| 045 | 8.9 |
| 046 | 6.2 |
| 047 | 7.0 |
| 048 | 12.9 |
| 049 | 6.2 |
| 051 | 13.1 |
| 052 | 6.4 |
| 053 | 12.2 |
| 054 | 6.4 |
| 055 | 7.8 |
| 057 | 11.3 |
| 060 | 6.2 |
| 061 | 6.2 |
| 062 | 6.2 |
| 063 | 10.2 |

1. A compound of formula (I), 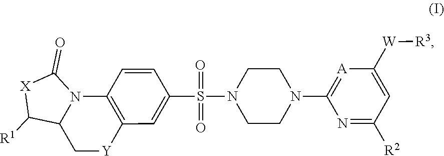
wherein
X is C₁₋₆alkylene, O, S or NH;
Y is C₁₋₆alkylene, O, S, SO, SO₂ or NH;
A is CH or N;
W is C₁₋₆alkylene, haloC₁₋₆alkylene, O, S, SO, SO₂ or NH;
R¹ is H, (aminoheterocyclyl)C₁₋₆alkyl, [(aminoC₁₋₆alkyl)heterocyclyl]C₁₋₆alkyl, amino, aminoC₁₋₆alkyl, hydroxyC₁₋₆alkyl or heterocyclylamino;
R² is amino, C₁₋₆alkyl, halogen, haloC₁₋₆alkyl, C₁₋₆alkoxy or haloC₁₋₆alkoxy;
R³ is (aminoC₁₋₆alkylamino)carbonylC₂₋₆alkynyl,
C₃₋₇cycloalkyl substituted by (aminoC₁₋₆alkyl)₂aminocarbonyl, [(aminoC₁₋₆alkyl)pyrrolidiniumyl]C₁₋₆alkylamino, [(aminocarbonylC₁₋₆alkyl)pyrrolidiniumylC₁₋₆alkyl]amino, [aminocarbonylC₁₋₆alkyl(C₁₋₆alkyl)pyrrolidiniumylcarbonyl]amino, amino, aminoC₁₋₆alkylaminocarbonyl or aminoC₁₋₆alkylcarbonylamino,
halogen,
heterocyclyl unsubstituted or substituted by amino, C₁₋₆alkyl, aminoC₁₋₆alkylcarbonyl, (aminoC₁₋₆alkylcarbonyl)aminoC₁₋₆alkylcarbonyl, aminoC₁₋₆alkyl or aminocarbonylC₁₋₆alkyl, or
aryl or heteroaryl unsubstituted or substituted by amino, C₁₋₆alkyl, halogen, haloC₁₋₆alkyl, C₁₋₆alkoxy or haloC₁₋₆alkoxy,
or a pharmaceutically acceptable salt thereof.

2. A compound according to claim 1, wherein
X is C₁₋₆alkylene or O;
Y is O;
A is CH or N;
W is haloC₁₋₆alkylene, O or SO₂;
R¹ is H, (aminoazetidinyl)C₁₋₆alkyl, [(aminoC₁₋₆alkyl)azetidinyl]C₁₋₆alkyl, amino, aminoC₁₋₆alkyl, hydroxyC₁₋₆alkyl or piperidylamino;
R² is C₁₋₆alkyl or halogen;
R³ is (aminoC₁₋₆alkylamino)carbonylC₂₋₆alkynyl,
1,4-dioxanyl,
3,6-dihydro-2H-pyridinyl substituted by C₁₋₆alkyl,
3-azabicyclo[3.1.0]hexanyl,
3-oxabicyclo[3.1.0]hexanyl,
azetidinyl substituted by aminoC₁₋₆alkylcarbonyl,
C₃₋₇cycloalkyl substituted by (aminoC₁₋₆alkyl)₂aminocarbonyl, [(aminoC₁₋₆alkyl)pyrrolidiniumyl]C₁₋₆alkylamino, [(aminocarbonylC₁₋₆alkyl)pyrrolidiniumylC₁₋₆alkyl]amino, [aminocarbonylC₁₋₆alkyl(C₁₋₆alkyl)pyrrolidiniumylcarbonyl]amino, amino, aminoC₁₋₆alkylaminocarbonyl or aminoC₁₋₆alkylcarbonylamino,
halogen,
morpholinyl unsubstituted or substituted by C₁₋₆alkyl,
phenyl substituted by amino, or
piperidyl unsubstituted or substituted by (aminoC₁₋₆alkylcarbonyl)aminoC₁₋₆alkylcarbonyl, aminoC₁₋₆alkyl, aminoC₁₋₆alkylcarbonyl, aminocarbonylC₁₋₆alkyl or C₁₋₆alkyl;
or a pharmaceutically acceptable salt thereof.

3. A compound according to claim 1 or 2, wherein X is CH₂ or O.

4. A compound according to any one of claims 1-3, wherein W is haloC₁₋₆alkylene.

5. A compound according to any one of claims 1-4, wherein W is CF₂.

6. A compound according to any one of claims 1-5, wherein R¹ is (aminoazetidinyl)C₁₋₆alkyl, amino, aminoC₁₋₆alkyl or hydroxyC₁₋₆alkyl.

7. A compound according to any one of claims 1-6, wherein R¹ is (3-aminoazetidin-1-yl)methyl, amino, aminomethyl or hydroxymethyl.

8. A compound according to any one of claims 1-7, wherein R² is chloro or methyl.

9. A compound according to any one of claims 1-8, wherein
R³ is 1,4-dioxanyl,
3,6-dihydro-2H-pyridinyl substituted by C₁₋₆alkyl,
3-azabicyclo[3.1.0]hexanyl,
C₃₋₇cycloalkyl substituted by (aminoC₁₋₆alkyl)₂aminocarbonyl, [aminocarbonylC₁₋₆alkyl(C₁₋₆alkyl)pyrrolidiniumylcarbonyl]amino or aminoC₁₋₆alkylaminocarbonyl,
halogen,
morpholinyl, or
piperidyl.

10. A compound according to any one of claims 1-9, wherein R³ is 1,4-dioxan-2-yl, 1-methyl-3,6-dihydro-2H-pyridin-4-yl, 3-azabicyclo[3.1.0]hexan-1-yl, 4-(3-aminopropylcarbamoyl)cyclohexyl, 4-(3-aminopropylcarbamoyl)cyclohexyl, 4-(3-aminopropylcarbamoyl)cyclohexyl, 4-[[1-(2-amino-2-oxo-ethyl)-1-methyl-pyrrolidin-1-ium-2-carbonyl]amino]cyclohexyl, 4-[bis(3-aminopropyl)carbamoyl]cyclohexyl, 4-piperidyl, fluoro or morpholin-2-yl.

11. A compound according to any one of claims 1-10, wherein
X is C₁₋₆alkylene or O;
Y is O;
A is CH or N;
W is haloC₁₋₆alkylene;
R¹ is (aminoazetidinyl)C₁₋₆alkyl, amino, aminoC₁₋₆alkyl or hydroxyC₁₋₆alkyl;
R² is C₁₋₆alkyl or halogen;
R³ is 1,4-dioxanyl,
3,6-dihydro-2H-pyridinyl substituted by C₁₋₆alkyl,
3-azabicyclo[3.1.0]hexanyl,
C₃₋₇cycloalkyl substituted by (aminoC₁₋₆alkyl)₂aminocarbonyl, [aminocarbonylC₁₋₆alkyl(C₁₋₆alkyl)pyrrolidiniumylcarbonyl]amino or aminoC₁₋₆alkylaminocarbonyl,
halogen,
morpholinyl, or
piperidyl;
or a pharmaceutically acceptable salt thereof.

12. A compound according to any one of claims 1-11, wherein
X is CH₂ or O;
Y is O;
A is CH or N;
W is CF₂;
R¹ is (3-aminoazetidin-1-yl)methyl, amino, aminomethyl or hydroxymethyl;
R² is chloro or methyl;
R³ is 1,4-dioxan-2-yl, 1-methyl-3,6-dihydro-2H-pyridin-4-yl, 3-azabicyclo[3.1.0]hexan-1-yl, 4-(3-aminopropylcarbamoyl)cyclohexyl, 4-(3-aminopropylcarbamoyl)cyclohexyl, 4-(3-aminopropylcarbamoyl)cyclohexyl, 4-[[1-(2-amino-2-oxo-ethyl)-1-methyl-pyrrolidin-1-ium-2-carbonyl]amino]cyclohexyl, 4-[bis(3-aminopropyl)carbamoyl]cyclohexyl, 4-piperidyl, fluoro or morpholin-2-yl;
or a pharmaceutically acceptable salt thereof.

13. A compound selected from:
trans-3-(aminomethyl)-7-[4-[6-chloro-4-(trifluoromethyl)-2-pyridyl]piperazin-1-yl]sulfonyl-3a,4-dihydro-3H-oxazolo[4,3-c][1,4]benzoxazin-1-one;
trans-3-(aminomethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
trans-3-(aminomethyl)-7-[4-(6-chloro-4-cyclopropylsulfonyl-2-pyridyl) piperazin-1-yl]sulfonyl-3a,4-dihydro-3H-oxazolo[4,3-c][1,4]benzoxazin-1-one;
trans-3-(aminomethyl)-7-[4-[4-(4-aminophenyl)sulfonyl-6-chloro-2-pyridyl]piperazin-1-yl]sulfonyl-3a,4-dihydro-3H-oxazolo[4,3-c][1,4]benzoxazin-1-one;
cis-3-(aminomethyl)-7-[4-(6-chloro-4-cyclopropylsulfonyl-2-pyridyl) piperazin-1-yl]sulfonyl-3a,4-dihydro-3H-oxazolo[4,3-c][1,4]benzoxazin-1-one;
cis-3-(aminomethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
cis-3-(aminomethyl)-7-[4-[6-chloro-4-(trifluoromethyl)-2-pyridyl]piperazin-1-yl]sulfonyl-3a,4-dihydro-3H-oxazolo[4,3-c][1,4]benzoxazin-1-one;
3-Amino-7-[4-[6-chloro-4-[1,4-dioxan-2-yl(difluoro)methyl]-2-pyridyl]piperazin-1-yl]sulfonyl-2,3,3a,4-tetrahydropyrrolo[2,1-c][1,4]benzoxazin-1-one;
trans-3-(aminomethyl)-7-[4-[6-chloro-4-[difluoro(3-piperidyl)methyl]-2-pyridyl]piperazin-1-yl]sulfonyl-3a,4-dihydro-3H-oxazolo[4,3-c][1,4]benzoxazin-1-one;
cis-3-(aminomethyl)-7-[4-[6-chloro-4-[difluoro-(1-methyl-4-piperidyl)methyl]-2-pyridyl]piperazin-1-yl]sulfonyl-3a,4-dihydro-3H-oxazolo[4,3-c][1,4]benzoxazin-1-one;
7-[4-[4-Methyl-6-(trifluoromethyl)pyrimidin-2-yl]piperazin-1-yl]sulfonyl-2,3,3a,4-tetrahydropyrrolo[2,1-c][1,4]benzoxazin-1-one;
7-[4-[6-Chloro-4-(trifluoromethyl)-2-pyridyl]piperazin-1-yl]sulfonyl-2,3,3a,4-tetrahydropyrrolo[2,1-c][1,4]benzoxazin-1-one;
7-[4-[4-(4-Aminophenyl)sulfonyl-6-chloro-2-pyridyl]piperazin-1-yl]sulfonyl-2,3,3a,4-tetrahydropyrrolo[2,1-c][1,4]benzoxazin-1-one;
7-[4-[4-(4-Aminophenyl)sulfonyl-6-bromo-2-pyridyl]piperazin-1-yl]sulfonyl-2,3,3a,4-tetrahydropyrrolo[2,1-c][1,4]benzoxazin-1-one;
(3R,3aR)-3-(hydroxymethyl)-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
7-[4-[6-Chloro-4-[difluoro-(1-methyl-4-piperidyl)methyl]-2-pyridyl]piperazin-1-yl]sulfonyl-2,3,3a,4-tetrahydropyrrolo[2,1-c][1,4]benzoxazin-1-one;
trans-3-(aminomethyl)-7-[4-[6-chloro-4-[1,4-dioxan-2-yl(difluoro)methyl]-2-pyridyl]piperazin-1-yl]sulfonyl-3a,4-dihydro-3H-oxazolo[4,3-c][1,4]benzoxazin-1-one;
trans-3-(aminomethyl)-7-[4-[6-chloro-4-[difluoro(3-oxabicyclo[3.1.0]hexan-6-yl)methyl]-2-pyridyl]piperazin-1-yl]sulfonyl-3a,4-dihydro-3H-oxazolo[4,3-c][1,4]benzoxazin-1-one;
trans-3-(aminomethyl)-7-[4-[6-chloro-4-[difluoro-(2-methyl-4-pyridyl)methyl]-2-pyridyl]piperazin-1-yl]sulfonyl-3a,4-dihydro-3H-oxazolo[4,3-c][1,4]benzoxazin-1-one;
cis-3-(aminomethyl)-7-[4-[6-chloro-4-[difluoro-(1-methyl-3,6-dihydro-2H-pyridin-4-yl)methyl]-2-pyridyl]piperazin-1-yl]sulfonyl-3a,4-dihydro-3H-oxazolo[4,3-c][1,4]benzoxazin-1-one;
(3R,3aS)-3-(Aminomethyl)-7-[4-[6-chloro-4-[[(2S)-1,4-dioxan-2-yl]-difluoro-methyl]-2-pyridyl]piperazin-1-yl]sulfonyl-3a,4-dihydro-3H-oxazolo[4,3-c][1,4]benzoxazin-1-one;
(3S,3aR)-3-(aminomethyl)-7-[4-[6-chloro-4-[[(2S)-1,4-dioxan-2-yl]-difluoro-methyl]-2-pyridyl]piperazin-1-yl]sulfonyl-3a,4-dihydro-3H-oxazolo[4,3-c][1,4]benzoxazin-1-one;
(3S,3aS)-3-(aminomethyl)-7-[4-[6-chloro-4-[difluoro-[(2R)-morpholin-2-yl]methyl]-2-pyridyl]piperazin-1-yl]sulfonyl-3a,4-dihydro-3H-oxazolo[4,3-c][1,4]benzoxazin-1-one;
(3R,3aR)-3-(aminomethyl)-7-[4-[6-chloro-4-[difluoro-[(2R)-morpholin-2-yl]methyl]-2-pyridyl]piperazin-1-yl]sulfonyl-3a,4-dihydro-3H-oxazolo[4,3-c][1,4]benzoxazin-1-one;
(3R,3aS)-3-[(3-aminoazetidin-1-yl)methyl]-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
(3R,3aS)-3-[[3-(aminomethyl)azetidin-1-yl]methyl]-7-[4-[4-methyl-6-(trifluoromethyl)pyrimidin-2-yl]piperazin-1-yl]sulfonyl-3a,4-dihydro-3H-oxazolo[4,3-c][1,4]benzoxazin-1-one;
7-[4-[6-Chloro-4-[difluoro-[rac-(2S)-1,4-dioxan-2-yl]methyl]-2-pyridyl]piperazin-1-yl]sulfonyl-3-(4-piperidylamino)-2,3,3a,4-tetrahydropyrrolo[2,1-c][1,4]benzoxazin-1-one;
(3R,3aR)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3S,3aS)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
7-[4-[6-Chloro-4-[difluoro-[(2R)-morpholin-2-yl]methyl]-2-pyridyl]piperazin-1-yl]sulfonyl-2,3,3a,4-tetrahydropyrrolo[2,1-c][1,4]benzoxazin-1-one;
3-Amino-7-[4-[6-chloro-4-[difluoro-[(2R)-morpholin-2-yl]methyl]-2-pyridyl]piperazin-1-yl]sulfonyl-2,3,3a,4-tetrahydropyrrolo[2,1-c][1,4]benzoxazin-1-one;
cis-7-[4-[6-chloro-4-[difluoro-[(2R)-morpholin-2-yl]methyl]-2-pyridyl]piperazin-1-yl]sulfonyl-3-(hydroxymethyl)-3a,4-dihydro-3H-oxazolo[4,3-c][1,4]benzoxazin-1-one;
cis-7-[4-[6-Chloro-4-[difluoro(3-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[3-azabicyclo[3.1.0]hexan-6-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[azetidin-3-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[3-azabicyclo[3.1.0]hexan-1-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3S,3aR)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aS)-7-[4-[6-chloro-4-[difluoro(4-piperidyl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[2-azaspiro[3.3]heptan-6-yl(difluoro)methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aS)-7-[4-[6-chloro-4-[difluoro-(5-methylmorpholin-2-yl)methyl]-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(2-aminopropanoyl)azetidin-3-yl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-(4-aminocyclohexoxy)-6-chloro-2-pyridyl]piperazin-1-yl]sulfonyl-3-(hydroxymethyl)-3a,4-dihydro-3H-oxazolo[4,3-c][1,4]benzoxazin-1-one;
3-Amino-7-[4-[4-(4-aminocyclohexoxy)-6-chloro-2-pyridyl]piperazin-1-yl]sulfonyl-2,3,3a,4-tetrahydropyrrolo[2,1-c][1,4]benzoxazin-1-one;
(E)-4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-N-(3-aminopropyl)-4,4-difluoro-but-2-enamide;
4-[[2-[4-[(3-Amino-1-oxo-2,3,3a,4-tetrahydropyrrolo[2,1-c][1,4]benzoxazin-7-yl)sulfonyl]piperazin-1-yl]-6-chloro-4-pyridyl]-difluoro-methyl]-N-(3-aminopropyl)cyclohexanecarboxamide;
4-[[2-[4-[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-(3-aminopropyl)cyclohexanecarboxamide;
4-[[2-[4-[[cis-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-(3-aminopropyl)cyclohexanecarboxamide;
4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N,N-bis(3-aminopropyl)cyclohexanecarboxamide;
4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-(3-aminopropyl)cyclohexanecarboxamide;
trans-7-[4-[6-chloro-4-[difluoro-[(2S)-morpholin-2-yl]methyl]-2-pyridyl]piperazin-1-yl]sulfonyl-3-(hydroxymethyl)-3a,4-dihydro-3H-oxazolo[4,3-c][1,4]benzoxazin-1-one;
N-[4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]oxy]cyclohexyl]-3-amino-propanamide;
(3R,3aR)-7-[4-[4-[[1-[2-[bis(2-aminoethyl)amino]acetyl]-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(2-aminoacetyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(3-aminopropanoyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
(3R,3aR)-7-[4-[4-[[1-(2-aminoethyl)-4-piperidyl]-difluoro-methyl]-6-chloro-2-pyridyl]piperazin-1-yl]sulfonyl-3-(hydroxymethyl)-3a,4-dihydro-3H-oxazolo[4,3-c][1,4]benzoxazin-1-one;
2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]acetamide;
N-[2-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-1-piperidyl]-2-oxo-ethyl]-2-amino-acetamide;
(1S,2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(1R,2S)—N-[4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(1S,2S)—N-[4-[[2-[4-[[(3S,3aR)-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(1R,2S)—N-[4-[[2-[4-[[(3S,3aR)-3-(aminomethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]cyclohexyl]-1-(2-amino-2-oxo-ethyl)-1-methyl-pyrrolidin-1-ium-2-carboxamide;chloride;
(2S)-1-(2-aminoethyl)-N-[4-[[2-chloro-6-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-4-pyridyl]-difluoro-methyl]cyclohexyl]-1-methyl-pyrrolidin-1-ium-2-carboxamide;chloride;
4-[[2-[4-[[(3R,3aR)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-amino-2-oxo-ethyl)pyrrolidin-1-ium-1-yl]ethyl]cyclohexanecarboxamide;chloride; and
4-[[2-[4-[[(3R,3aS)-3-(hydroxymethyl)-1-oxo-3a,4-dihydro-3H-oxazolo[4,3-c][1,4]benzoxazin-7-yl]sulfonyl]piperazin-1-yl]-6-chloro-4-pyridyl]-difluoro-methyl]-N-[2-[1-(2-aminoethyl)pyrrolidin-1-ium-1-yl]ethyl]cyclohexanecarboxamide;chloride;
or a pharmaceutically acceptable salt thereof.

14. A process for the preparation of a compound according to any one of claims 1 to 13 comprising any of the following steps:
a) formation of compound of formula (I) via substitution reaction between compound of formula (VII), 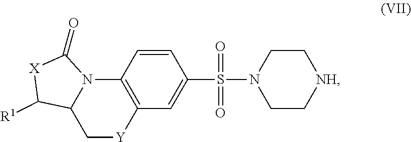
and compound of formula (VIII), 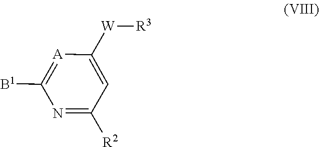
in the presence of a base;
b) formation of compound of formula (XVII), 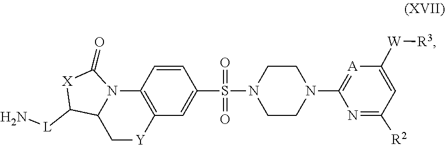
by deprotection of compound of formula (XVI), 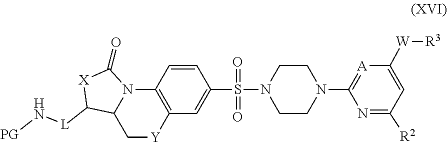
in the presence of an acid or a reducing reagent;
wherein
in step a) the base is DIEA;
in step b), the acid is trifluoroacetic acid; the reducing reagent is palladium on carbon in the presence of H₂;
wherein L is C₁₋₆alkylene or a bond; PG is tert-butoxycarbonyl or benzyloxycarbonyl; R¹ to R³, X, Y, A and W are defined as in any one of claims 1 to 12.

15. A compound or pharmaceutically acceptable salt according to any one of claims 1 to 13 for use as therapeutically active substance.

16. A pharmaceutical composition comprising a compound in accordance with any one of claims 1 to 13 and a therapeutically inert carrier.

17. The use of a compound according to any one of claims 1 to 13 for the inhibition of LpxH.

18. The use of a compound according to any one of claims 1 to 13 for the treatment or prophylaxis of bacterial infection, particularly the bacteria is gram-negative bacteria.

19. The use of a compound according to any one of claims 1 to 13 for the preparation of a medicament for the treatment or prophylaxis of bacterial infection, particularly the bacteria is gram-negative bacteria.

20. The use of a compound according to claim 18 or 19, wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

21. The use of a compound according to claim 20, wherein the gram-negative bacteria is Enterobacteriaceae, wherein Enterobacteriaceae is Klebsiella pneumoniae or Escherichia coli.

22. A compound or pharmaceutically acceptable salt according to any one of claims 1 to 13 for use in the treatment or prophylaxis of bacterial infection, particularly the bacteria is gram-negative bacteria.

23. A compound according to claim 22, wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

24. A compound according to claim 23, wherein the gram-negative bacteria is Enterobacteriaceae, wherein Enterobacteriaceae is Klebsiella pneumoniae or Escherichia coli.

25. A compound or pharmaceutically acceptable salt according to any one of claims 1 to 13, when manufactured according to a process of claim 14.

26. A method for the treatment or prophylaxis of bacterial infection, particularly the bacteria is gram-negative bacteria, which method comprises administering a therapeutically effective amount of a compound as defined in any one of claims 1 to 13, to a patient in need thereof.
